(19) United States
(12) Patent Application Publication
(10) Pub. No.: US 2011/0224227 A1
(43) Pub. Date: Sep. 15, 2011

(54) HEMATOPOIETIC PROTECTION AGAINST CHEMOTHERAPEUTIC COMPOUNDS USING SELECTIVE CYCLIN-DEPENDENT KINASE 4/6 INHIBITORS

(76) Inventors: Norman E. Sharpless, Chapel Hill, NC (US); Jay C. Strum, Hillsborough, NC (US); John E. Bisi, Apex, NC (US); Patrick J. Roberts, Durham, NC (US); Matthew R. Ramsey, Chapel Hill, NC (US)

(21) Appl. No.: 13/122,061

(22) PCT Filed: Oct. 1, 2009
(86) PCT No.: PCT/US09/59281

§ 371 Date: May 18, 2011

Related U.S. Application Data
(60) Provisional application No. 61/101,841, filed on Oct. 1, 2008.

(51) Int. Cl. A61K 31/497; A61K 31/519; A61K 31/505; A61K 31/4709; G01N 33/53; A61P 39/00

(52) U.S. Cl. 514/253.04; 514/258.1; 514/256; 514/314; 435/29

(57) ABSTRACT

Methods for reducing or preventing the effects of cytotoxic compounds in healthy cells are provided. The methods relate to the use of selective cyclin-dependent kinase (CDK) 4/6 inhibitors to induce transient quiescence in CDK4/6 dependent cells, such as hematopoietic stem cells and/or hematopoietic progenitor cells. Also described is a method of selecting compounds for reducing or preventing the effects of cytotoxic agents compounds in healthy cells.

### RELATED APPLICATIONS

The presently disclosed subject matter is based on and claims the benefit of U.S. Provisional Application Ser. No. 61/101,841, filed Oct. 1, 2008; the disclosure of which is incorporated herein by reference in its entirety.

### GOVERNMENT INTEREST

This presently disclosed subject matter was made with U.S. Government support under Grant Nos. RO1 AG024379-01 and K08 CA90679 awarded by the National Institutes of Health through the National Institute on Aging and the National Cancer Institute. Thus, the U.S. Government has certain rights in the presently disclosed subject matter.

### TECHNICAL FIELD

The presently disclosed subject matter relates to methods of protecting healthy cells from damage due to cytotoxic compounds, such as DNA damaging compounds. In particular, the presently disclosed subject matter relates to the protective action of cyclin-dependent kinase 4 (CDK4) and/or cyclin-dependent 6 (CDK6) inhibitors administered to subjects that have been exposed to, that are being or will be exposed to, or that are at risk of exposure to cytotoxic compounds.

### ABBREVIATIONS

° C.=degrees Celsius
%=percentage
μL=microliters
μM=micromolar
2BrIC=2-bromo-12,13-dihydro-5H-indolo[2,3-a]pyrrolo[3,4]-carbazole-5,6-dione
BM=bone marrow
BM-MNC=bone marrow mononuclear cells
BrdU=5-bromo-2-deoxyuridine
Carbo=carboplatin
CBC=complete blood count
CDK=cyclin-dependent kinase
CDK4/6=cyclin dependent kinase 4 and/or cyclin-dependent kinase 6
CLP=common lymphoid progenitors
CMP=common myeloid progenitors
CNS=central nervous system
DMEM=Dulbecco's Modified Eagle Medium
DMF=dimethylformamide
DNA=deoxyribonucleic acid
DOX=doxorubicin
EPO=erythropoietin
ESI=electrospray ionization
EtOAc=ethyl acetate
EtOH=ethanol
Etop=etoposide
FBS=fetal bovine serum
g=gram
G-CSF=granulocyte colony stimulating factor
GM-CSF=granulocyte-macrophage colony stimulating factor
GMP=granulocyte-monocyte progenitors
h=hours
HSC=hematopoietic stem cells
HSPC=hematopoietic stem and progenitor cells
IC₅₀=50% inhibitory concentration
i.p.=intraperitoneal
kg=kilogram
LC-MS=liquid chromatography-mass spectroscopy
LT-HSC=long term hematopoietic stem cell
M=molar
MEP=megakaryocyte-erythroid progenitors
mg=milligrams
MHz=megaHertz
mL=milliliters
mmol=millimoles
mol=moles
Mp=melting point
Mpk=milligrams per kilogram
MPP=multipotent progenitor
NBS=N-bromosuccinimide
nm=nanometer
nM=nanomolar
NMR=nuclear magnetic resonance
PD=6-acetyl-8-cyclopentyl-5-methyl-2-(5-piperazin-1-yl-pyridin-2-ylamino)-8H-pyrido-[2,3-d]-pyrimidin-7-one (also referred to as PD 0332991)
PI=propidium iodide
PQ=pharmacologic quiescence
RB=retinoblastoma tumor suppressor protein
RLU=relative light units
r.t.=room temperature
ST-HSC=short term hematopoietic stem cell
tHDF=telomerized human diploid fibroblast
THF=tetrahydrofuran
UV=ultraviolet

### BACKGROUND

Chemotherapy refers to the use of cytotoxic (e.g., DNA damaging) drugs such as, but not limited to busulfan, cyclophosphamide, doxorubicin, daunorubicin, vinblastine, vincristine, bleomycin, etoposide, topotecan, irinotecan, taxotere, taxol, 5-fluorouracil, methotrexate, gemcitabine, cisplatin, carboplatin or chlorambucil in order to eradicate cancer cells and tumors. Chemotherapuetic compounds tend to be non-specific and, particularly at high doses, toxic to normal, rapidly dividing cells. This often leads to various side effects in patients undergoing chemotherapy.

Bone marrow suppression, a severe reduction of blood cell production in bone marrow, is one such side effect. It is characterized by both myelosuppression (anemia, neutropenia, agranulocytosis and thrombocytopenia) and lymphopenia. Neutropenia is characterized by a selective decrease in the number of circulating neutrophils and an enhanced susceptibility to bacterial infections. Anemia, a reduction in the number of red blood cells or erythrocytes, the quantity of hemoglobin, or the volume of packed red blood cells (characterized by a determination of the hematocrit) affects approximately 67% of cancer patients undergoing chemotherapy in the United States. See BioWorld Today, page 4, Jul. 23, 2002. The cytotoxicity of chemotherapeutic agents limits administrable doses, affects treatment cycles and seriously jeopardizes the quality of life for the cancer patient. Thrombcytopenia is a reduction in platelet number with increased susceptibility to bleeding. Lymphopenia is a common side-effect of chemotherapy characterized by reductions in the numbers of circulating lymphocytes (also called T- and B-cells). Lymphopenic patients are predisposed to a number of types of infections.

Small molecules have been used to reduce some of the side effects of certain chemotherapeutic compounds. For example, leukovorin has been used to mitigate the effects of methotrexate on bone marrow cells and on gastrointestinal mucosa cells. Amifostine has been used to reduce the incidence of neutropenia-related fever and mucositis in patients receiving alkylating or platinum-containing chemotherapeutics. Also, dexrazoxane has been used to provide cardioprotection from anthracycline anti-cancer compounds. Unfortunately, there is concern that many chemoprotectants, such as dexrazoxane and amifostine, can decrease the efficacy of chemotherapy given concomitantly.

Additional chemoprotectant therapies, particularly with regard to chemotherapy associated anemia and neutropenia, include the use of growth factors. Hematopoietic growth factors are available on the market as recombinant proteins. These proteins include granulocyte colony stimulating factor (G-CSF) and granulocyte-macrophage colony stimulating factor (GM-CSF) and their derivatives for the treatment of neutropenia, and erythropoietin (EPO) and its derivatives for the treatment of anemia. However, these recombinant proteins are expensive. Moreover, EPO has significant toxicity in cancer patients, leading to increased thrombosis, relapse and death in several large randomized trials. G-CSF and GM-CSF may increase the late (>2 years post-therapy) risk of secondary bone marrow disorders such as leukemia and myelodysplasia. Consequently, their use is restricted and not readily available to all patients in need. Further, while growth factors can hasten recovery of some blood cell lineages, no therapy exists to treat suppression of platelets, macrophages, T-cells or B-cells.

The non-selective kinase inhibitor staurosporine has been shown to afford protection from DNA damaging agents in some cultured cell types. See Chen et al., J. Natl. Cancer Inst., 92, 1999-2008 (2000); and Ojeda et al., Int. J. Radiat. Biol., 61, 663-667 (1992). Staurosporine is a naturally occurring product and non-selective kinase inhibitor that binds most mammalian kinases with high affinity. See Karaman et al., Nat. Biotechnol., 26, 127-132 (2008). Staurosporine treatment can elicit an array of cellular responses including apoptosis, cell cycle arrest and cell cycle checkpoint compromise depending on cell type, drug concentration, and length of exposure. For example, staurosporine has been shown to sensitize cells to DNA damaging agents such as ionizing radiation and chemotherapy (see Bernhard et al., Int. J. Radiat. Biol., 69, 575-584 (1996); Teyssier et al., Bull. Cancer, 86, 345-357 (1999); Hallahan et al., Radiat. Res., 129, 345-350 (1992); Zhang et al., J. Neurooncol., 15, 1-7 (1993); Guo et al., Int. J. Radiat. Biol., 82, 97-109 (2006); Bucher and Britten, Br. J. Cancer, 98, 523-528 (2008); Laredo et al., Blood, 84, 229-237 (1994); Luo et al., Neoplasia, 3, 411-419 (2001); Wang et al., Yao Xue Xue Bao, 31, 411-415 (1996); Chen et al., J. Natl. Cancer Inst., 92, 1999-2008 (2000); and Hirose et al., Cancer Res., 61, 5843-5849 (2001)) through several claimed mechanisms including abrogation of a G2 checkpoint response. The mechanism whereby staurosporine treatment affords protection from DNA damaging agents in some cultured cell types is unclear, with a few possible mechanisms suggested including inhibition of protein kinase C or decreasing CDK4 protein levels. See Chen et al., J. Natl. Cancer Inst., 92, 1999-2008 (2000); and Ojeda et al., Int. J. Radiat. Biol., 61, 663-667 (1992). No effect of staurosporine has been shown on hematopoietic progenitors, nor has staurosporine use well after exposure to DNA damaging agents been shown to afford protection. Staurosporine's non-selective kinase inhibition has led to significant toxicities independent of its effects on the cell cycle (e.g. hyperglycemia) after in vivo administration to mammals and these toxicities have precluded its clinical use.

Given these deficiencies of the above-mentioned methods, there is an ongoing need for practical methods to protect subjects who are incurring, or who are scheduled to incur, exposure to chemotherapy. There is a particular need to protect chemotherapy patients from myelosuppression and lymphopenia. Further, chemoprotectant strategies are needed that do not reduce the efficacy of the chemotherapy on cancer cells.

### SUMMARY

The presently disclosed subject matter provides, in some embodiments, a method of reducing or preventing the effects of a cytotoxic compound on healthy cells in a subject who has been exposed to, shall be exposed to, or is at risk of incurring exposure to a cytotoxic compound, wherein said healthy cells are hematopoietic stem cells or hematopoietic progenitor cells, the method comprising administering to the subject an effective amount of an inhibitor compound, or a pharmaceutically acceptable form thereof, wherein the inhibitor compound selectively inhibits cyclin-dependent kinase 4 (CDK4) and/or cyclin-dependent kinase 6 (CDK6).

In some embodiments, the inhibitor compound selectively inhibits both CDK4 and CDK6. In some embodiments, the inhibitor compound is a non-naturally occurring compound.

In some embodiments, the inhibitor compound is substantially free of off-target effects. In some embodiments, the off-target effects are one or more of the group consisting of long term toxicity, anti-oxidant effects, estrogenic effects, tyrosine kinase inhibition, inhibition of cyclin-dependent kinases (CDKs) other than CDK4/6, and cell cycle arrest in CDK4/6-independent cells.

In some embodiments, the inhibitor compound selectively induces G1 arrest in CDK4/6-dependent cells. In some embodiments, the inhibitor compound induces substantially pure G1 arrest in CDK4/6-dependent cells.

In some embodiments, the inhibitor compound is selected from the group consisting of a pyrido[2,3-d]pyrimidine, a triaminopyrimidine, an aryl[a]pyrrolo[3,4-c]carbazole, a nitrogen-containing heteroaryl-substituted urea, a 5-pyrimidinyl-2-aminothiazole, a benzothiadiazine, and an acridinethione.

In some embodiments, the pyrido[2,3-d]pyrimidine is a pyrido[2,3-d]pyrimidin-7-one or a 2-amino-6-cyano-pyrido[2,3-d]pyrimidin-4-one. In some embodiments, the pyrido[2,3-d]pyrimidin-7-one is a 2-(2′-pyridyl)amino pyrido[2,3-d]pyrimidin-7-one. In some embodiments, the pyrido[2,3-d]pyrimidin-7-one is 6-acetyl-8-cyclopentyl-5-methyl-2-(5-piperazin-1-yl-pyridin-2-ylamino)-8H-pyrido-[2,3-d]pyrimidin-7-one.

In some embodiments, the aryl[a]pyrrolo[3,4-c]carbazole is selected from the group consisting of a napthyl[a]pyrrolo[3,4-c]carbazole, an indolo[a]pyrrolo[3,4-c]carbazole, a quinolinyl[a]pyrrolo[3,4-c]carbazole, and an isoquinolinyl[a]pyrrolo[3,4-c]carbazole. In some embodiments, the aryl[a]pyrrolo[3,4-c]carbazole is 2-bromo-12,13-dihydro-5H-indolo[2,3-a]pyrrolo[3,4]-carbazole-5,6-dione.

In some embodiments, the subject is a mammal. In some embodiments, the inhibitor compound is administered to the subject by one of the group consisting of oral administration, topical administration, intranasal administration, inhalation, and intravenous administration.

In some embodiments, the inhibitor compound is administered to the subject prior to exposure to the cytotoxic compound, during exposure to the cytotoxic compound, after exposure to the cytotoxic compound or any combination thereof. In some embodiments, the inhibitor compound is administered to the subject 24 hours or less prior to exposure to the cytotoxic compound. In some embodiments, the inhibitor compound is administered to the subject 24 hours or more following exposure to the cytotoxic compound.

In some embodiments, the cytotoxic compound is a DNA damaging compound.

In some embodiments, the healthy cells are selected from the group consisting of long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs). In some embodiments, administration of the inhibitor compound provides temporary pharmacologic quiescence in hematopoietic stem and progenitor cells.

In some embodiments, the subject has undergone, is undergoing, or is expected to undergo medical treatment with a cytotoxic compound to treat a disease. In some embodiments, administration of the inhibitor compound does not affect growth of diseased cells.

In some embodiments, the disease is cancer. In some embodiments, the cancer is characterized by one or more of the group consisting of increased activity of cyclin-dependent kinase 1 (CDK1), increased activity of cyclin-dependent kinase 2 (CDK2), loss or absence of retinoblastoma tumor suppressor protein (RB), high levels of MYC expression, increased cyclin E and increased cyclin A.

In some embodiments, administration of the inhibitor compound allows for a higher dose of the cytotoxic compound to be used to treat the disease than the dose that would be used in the absence of administration of the inhibitor compound.

In some embodiments, the subject has been accidentally exposed to the cytotoxic compound or to an overdose of the cytotoxic compound.

In some embodiments, the method is free of long-term hematologic toxicity. In some embodiments, administration of the inhibitor compound results in reduced anemia, reduced lymphopenia, reduced thrombocytopenia, or reduced neutropenia compared to that expected after exposure to the cytotoxic compound in the absence of administration of the inhibitor compound.

In some embodiments, the presently disclosed subject matter provides a method for screening a compound for use in preventing the effects of a cytotoxic agent in a healthy cell, the method comprising: contacting a CDK4/6-dependent cell population with a test compound for a period of time; performing cell cycle analysis of the cell population; and selecting a test compound that selectively induces G1 arrest in the cell population.

In some embodiments, the CDK4/6-dependent cell population comprises telomerized human diploid fibroblast cells or melanoma cells lacking INK4a/ARF. In some embodiments, the cell cycle analysis is performed using one or more of the techniques selected from flow cytometry, fluorimetry, cell imaging, and fluorescence spectroscopy. In some embodiments, the cell cycle analysis comprises labeling the cell population with one or more labeling agents selected from the group consisting of 5-bromo-2-deoxyuridine (BrdU) and propidium iodide (PI).

In some embodiments, the method further comprises: contacting a second cell population with the test compound that selectively induces G1 arrest inCDK4/6-dependent cells for a period of time, wherein the second cell population comprises CDK4/6-independent cells; performing cell cycle analysis in the second cell population; and selecting a test compound that is free of selective induction of G1 arrest in the second cell population.

In some embodiments, the second cell population is a cancer cell line. In some embodiments, the second cell population is RB-null.

In some embodiments, the method further comprises confirming the preventative ability of the test compound by assessing the ability of the compound to reduce DNA damage, to maintain cell viability, or both in an ex vivo cell population contacted with a cytotoxic agent. In some embodiments, DNA damage in the cell population is assessed by performing a gamma-H2AX assay. In some embodiments, cell viability is assessed by performing a cell proliferation assay.

In some embodiments, the cytotoxic agent is a DNA damaging compound. In some embodiments, the DNA damaging compound is selected from the group consisting of doxorubicin, etoposide and carboplatin.

It is an object of the presently disclosed subject matter to provide methods of protecting healthy cells in subjects from the effects of DNA damaging compounds by administering to the subject an effective amount of a selective CDK4/6 inhibitor compound.

An object of the presently disclosed subject matter having been stated hereinabove, and which is achieved in whole or in part by the presently disclosed subject matter, other objects will become evident as the description proceeds when taken in connection with the accompanying drawings as best described hereinbelow.

### BRIEF DESCRIPTION OF THE DRAWINGS

FIG. 1 is a schematic drawing of hematopoiesis, the hierarchical proliferation of hematopoietic stem cells (HSC) and progenitor cells with increasing differentiation upon proliferation.

FIG. 2A is a set of representative histograms of cell cycle analysis of cyclin-dependent kinase 4/6 (CDK4/6)-dependent cells (human melanoma cells lacking INK4a/ARF; WM2664) treated for 24 hours with (from top to bottom) 0 nM, 15 nM, 30 nM, 89 nM, or 270 nM 6-acetyl-8-cyclopentyl-5-methyl-2-(5-piperazin-1-yl-pyridin-2-ylamino)-8H-pyrido-[2, 3-d]pyrimidin-7-one (PD 0332991). Data was fitted using Mod-Fit™ software (Varity Software House, Topsham, Me., United States of America).

FIG. 2B is a set of representative histograms of cell cycle analysis of cyclin-dependent kinase 4/6 (CDK4/6)-dependent cells (human melanoma cells lacking INK4a/ARF; WM2664) treated for 24 hours with (from top to bottom) 0 nM, 122 nM, 370 nM, 1.1 μM or 3.3 μM 2-bromo-12,13-dihydro-5H-indolo[2,3-a]pyrrolo[3,4]-carbazole-5,6-dione (2BrIC). Data was fitted using Mod-Fit™ software (Varity Software House, Topsham, Me., United States of America).

FIG. 2C is a graph showing the percentage (%) of cyclin-dependent kinase 4/6 (CDK4/6)-dependent cells (human melanoma cells lacking INK4a/ARF; WM2664) in the G1 cell cycle phase following treatment for 24 hours with 0 nM, 15 nM, 30 nM, 89 nM, or 270 nM 6-acetyl-8-cyclopentyl-5-methyl-2-(5-piperazin-1-yl-pyridn-2-ylamino)-8H-pyrido-[2,3-d]pyrimidin-7-one (PD 0332991) or 0 nM, 122 nM, 370 nM, 1.1 μM or 3.3 μM 2-bromo-12,13-dihydro-5H-indolo[2,3-a]pyrrolo[3,4]-carbazole-5,6-dione (2BrIC) as indicated.

FIG. 2D is a graph showing the percentage (%) of cyclin-dependent kinase 4/6 (CDK4/6)-dependent cells (human melanoma cells lacking INK4a/ARF; WM2664) in the G2/M cell cycle phase following treatment for 24 hours with 0 nM, 15 nM, 30 nM, 89 nM, or 270 nM 6-acetyl-8-cyclopentyl-5-methyl-2-(5-piperazin-1-yl-pyridin-2-ylamino)-8H-pyrido-[2,3-d]pyrimidin-7-one (PD 0332991) or 0 nM, 122 nM, 370 nM, 1.1 μM or 3.3 μM 2-bromo-12,13-dihydro-5H-indolo[2,3-a]pyrrolo[3,4]-carbazole-5,6-dione (2BrIC) as indicated.

FIG. 2E is a graph showing the percentage (%) of cyclin-dependent kinase 4/6 (CDK4/6)-dependent cells (human melanoma cells lacking INK4a/ARF; WM2664) in the S cell cycle phase following treatment for 24 hours with 0 nM, 15 nM, 30 nM, 89 nM, or 270 nM 6-acetyl-8-cyclopentyl-5-methyl-2-(5-piperazin-1-yl-pyridin-2-ylamino)-8H-pyrido-[2,3-d]pyrimidin-7-one (PD 0332991) or 0 nM, 122 nM, 370 nM, 1.1 μM or 3.3 μM 2-bromo-12,13-dihydro-5H-indolo[2,3-a]pyrrolo[3,4]-carbazole-5,6-dione (2BrIC) as indicated.

FIG. 3A is a bar graph showing the ability of 2-bromo-12,13-dihydro-5H-indolo[2,3-a]pyrrolo[3,4]-carbazole-5,6-dione (2BrIC) to protect cyclin-dependent kinase 4/6 (CDK4/6)-dependent cells from carboplatin-induced DNA damage. Human melanoma cells lacking INK4a/ARF (WM2664) were pretreated for 16 hours with 2BrIC followed by 8 hours with carboplatin. DNA damage was assessed using the gamma-H2AX assay as described herein. The percentage (%) of gamma-H2AX positive cells is shown for WM2664 treated with either carboplatin alone or with carboplatin following pretreatment with 0.122, 0.37, 1.1, or 3.3 μM 2BrIC.

FIG. 3B is a bar graph showing the ability of 2-bromo-12,13-dihydro-5H-indolo[2,3-a]pyrrolo[3,4]-carbazole-5,6-dione (2BrIC) to protect cyclin-dependent kinase 4/6 (CDK4/6)-dependent cells from etoposide-induced DNA damage. Human melanoma cells lacking INK4a/ARF (WM2664) were pretreated for 16 hours with 2BrIC followed by 8 hours with etoposide. DNA damage was assessed using the gamma-H2AX assay as described herein. The percentage (%) of gamma-H2AX positive cells is shown for WM2664 treated with either etoposide alone or with etoposide following pretreatment with 0.122, 0.37, 1.1, or 3.3 μM 2BrIC.

FIG. 3C is a bar graph showing the ability of 2-bromo-12,13-dihydro-5H-indolo[2,3-a]pyrrolo[3,4]-carbazole-5,6-dione (2BrIC) to protect cyclin-dependent kinase 4/6 (CDK4/6)-dependent cells from doxorubicin-induced DNA damage. Human melanoma cells lacking INK4a/ARF (WM2664) were pretreated for 16 hours with 2BrIC followed by 8 hours with doxorubicin. DNA damage was assessed using the gamma-H2AX assay as described herein. The percentage (%) of gamma-H2AX positive cells is shown for WM2664 treated with either doxorubicin alone or with doxorubicin following pretreatment with 0.122, 0.37, 1.1, or 3.3 μM 2BrIC.

FIG. 4 is a bar graph showing the ability of 2-bromo-12,13-dihydro-5H-indolo[2,3-a]pyrrolo[3,4]-carbazole-5,6-dione (2BrIC) to protect cyclin-dependent kinase 4/6 (CDK4/6)-dependent cells from doxorubicin-, carboplatin- or etoposide-induced DNA damage as determined by assessing gamma-H2AX levels. The percentage (%) of gamma-H2AX positive cells is shown for untreated telomerized human diploid fibroblast (tHDF) cells (HS68); for HS68 cells treated for 16 hours with 122 nM, 370 nM, 1.1 μM, or 3.3 μM 2BrIC, for HS68 cells treated with either carboplatin (Garbo), etoposide (Etop), or doxorubicin (Dox) alone for 8 hours; and for HS68 cells treated with either Garbo, Etop or Dox for 8 hours following pretreatment with 122 nM, 370 nM, 1.1 μM, or 3.3 μM 2BrIC for 16 hours.

FIG. 5 is a bar graph showing the inability of 2-bromo-12,13-dihydro-5H-indolo[2,3-a]pyrrolo[3,4]-carbazole-5,6-dione (2BrIC) to protect cyclin-dependent kinase 4/6 (CDK4/6) independent cells (human RB-null melanoma cells (A2058)) from doxorubicin-, carboplatin- or etoposide-induced DNA damage as determined by assessing gamma-H2AX levels. The percentage (%) of gamma-H2AX positive cells is shown for untreated A2058 cells; for A2058 cells treated with 122 nM, 370 nM, 1.1 μM, or 3.3 μM 2BrIC for 16 hours; for A2058 cells treated with either carboplatin (Garbo), etoposide (Etop), or doxorubicin (Dox) alone for 8 hours; and for A2058 cells treated with either Carbo, Etop or Dox for 8 hours following pretreatment with 122 nM, 370 nM, 1.1 μM, or 3.3 μM 2BrIC for 16 hours.

FIG. 6A is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect cyclin-dependent kinase 4/6 (CDK4/6)-dependent cells from carboplatin-induced DNA damage. Human melanoma cells lacking INK4a/ARF (WM2664) were pretreated for 16 hours with PD 0332991 followed by 8 hours with carboplatin. DNA damage was assessed using the gamma-H2AX assay as described herein. The percentage (%) of gamma-H2AX positive cells is shown for WM2664 treated with either carboplatin alone or with carboplatin following pretreatment with 15 nM, 30 nM, 89 nM, or 270 nM PD0332991.

FIG. 6B is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect cyclin-dependent kinase 4/6 (CDK4/6)-dependent cells from etoposide-induced DNA damage. Human melanoma cells lacking INK4a/ARF (WM2664) were pretreated for 16 hours with PD 0332991 followed by 8 hours with etoposide. DNA damage was assessed using the gamma-H2AX assay as described herein. The percentage (%) of gamma-H2AX positive cells is shown for WM2664 treated with either etoposide alone or with etoposide following pretreatment with 15 nM, 30 nM, 89 nM, or 270 nM PD 0332991.

FIG. 6C is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect cyclin-dependent kinase 4/6 (CDK4/6)-dependent cells from doxorubicin-induced DNA damage. Human melanoma cells lacking INK4a/ARF (WM2664) were pretreated for 16 hours with PD 0332991 followed by 8 hours with doxorubicin. DNA damage was assessed using the gamma-H2AX assay as described herein. The percentage (%) of gamma-H2AX positive cells is shown for WM2664 treated with either doxorubicin alone or with doxorubicin following pretreatment with 15 nM, 30 nM, 89 nM, or 270 nM PD 0332991.

FIG. 7 is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect cyclin-dependent kinase 4/6 (CDK4/6)-dependent cells from doxorubicin-, carboplatin- or etoposide-induced DNA damage as determined by assessing gamma-H2AX levels. The percentage (%) of gamma-H2AX positive cells is shown for untreated telomerized human diploid fibroblast (tHDF) cells (HS68); for HS68 cells treated for 16 hours with 15 nM, 30 nM, 89 nM, or 270 nM PD 0332991; for HS68 cells treated with either carboplatin (Carbo), etoposide (Etop), or doxorubicin (Dox) alone for 8 hours; and for HS68 cells treated with either Carbo, Etop or Dox for 8 hours following pretreatment with 15 nM, 30 nM, 89 nM, or 270 nM PD 0332991 for 16 hours.

FIG. 8 is a bar graph showing the inability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect cyclin-dependent kinase 4/6 (CDK4/6) independent cells (human RB-null melanoma cells (A2058)) from doxorubicin-, carboplatin- or etoposide-induced DNA damage as determined by assessing gamma-H2AX levels. The percentage (%) of gamma-H2AX positive cells is shown for untreated A2058 cells; for A2058 cells treated with 15 nM, 30 nM, 89 nM, or 270 nM PD0332991 for 16 hours; for A2058 cells treated with either carboplatin (Carbo), etoposide (Etop), or doxorubicin (Dox) alone for 8 hours; and for A2058 cells treated with either Carbo, Etop or Dox for 8 hours following pretreatment with 15 nM, 30 nM, 89 nM, or 270 nM PD 0332991 for 16 hours.

FIG. 9 is a bar graph showing the ability of 2-bromo-12,13-dihydro-5H-indolo[2,3-a]pyrrolo[3,4]-carbazole-5,6-dione (2BrIC) to protect human melanoma cells lacking INK4a/ARF (WM2664) from doxorubicin-induced cytotoxicity as determined by assessing cell viability using the WST-1 assay. Relative cell number was determined by following absorbance at 450 nm. Results are shown for cells treated with either 2BrIC alone (striped bars; at 0.0 μM, 0.120 μM, 0.370 μM, 1.1 μM, or 3.3 μM); 2BrIC (at 0.0 μM, 0.120 μM, 0.370 μM, 1.1 μM, or 3.3 μM) and doxorubicin (DOX; solid bars); or DOX alone (open bars).

FIG. 10 is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect human melanoma cells lacking INK4a/ARF (WM2664) from doxorubicin-induced cytotoxicity as determined by assessing cell viability using the WST-1 assay. Relative cell number was determined by following absorbance at 450 nm. Results are shown for cells treated with either PD 0332991 alone (striped bars; at 0.0 μM, 0.120 μM, 0.370 μM, 1.1 μM, or 3.3 μM); PD 0332991 (at 0.0 μM, 0.120 μM, 0.370 μM, 1.1 μM, or 3.3 82 M) and doxorubicin (DOX; solid bars); or DOX alone (open bars).

FIG. 11 is a bar graph showing the ability of 2-bromo-12,13-dihydro-5H-indolo[2,3-a]pyrrolo[3,4]-carbazole-5,6-dione (2BrIC) to protect telomerized human diploid fibroblast (tHDF) cells (HS68) from doxorubicin-induced cytotoxicity as determined by assessing cell viability using the WST-1 assay. Relative cell number was determined by following absorbance at 450 nm. Results are shown for cells treated with either 2BrIC alone (striped bars; at 0.0 μM, 0.120 μM, 0.370 μM, 1.1 μM, or 3.3 μM); 2BrIC (at 0.0 μM, 0.120 μM, 0.370 μM, 1.1 μM, or 3.3 μM) and doxorubicin (DOX; solid bars); or DOX alone (open bars).

FIG. 12 is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect telomerized human diploid fibroblast (tHDF) cells (HS68) from doxorubicin-induced cytotoxicity as determined by assessing cell viability using the WST-1 assay. Relative cell number was determined by following absorbance at 450 nm. Results are shown for cells treated with either PD 0332991 alone (striped bars; at 0.0μM, 0.120 μM, 0.370 μM, 1.1 μM, or 3.3 μM); PD 0332991 (at 0.0 μM, 0.120 μM, 0.370 μM, 1.1 μM, or 3.3 μM) and doxorubicin (DOX; solid bars); or DOX alone (open bars).

FIG. 13 is a bar graph showing the inability of 2-bromo-12,13-dihydro-5H-indolo[2,3-a]pyrrolo[3,4]-carbazole-5,6-dione (2BrIC) to protect human RB-null melanoma cells (A2058) from doxorubicin-induced cytotoxicity as determined by assessing cell viability using the WST-1 assay. Relative cell number was determined by following absorbance at 450 nm. Results are shown for cells treated with either 2BrIC alone (striped bars; at 0.0 μM, 0.120 μM, 0.370 μM, 1.1 μM, or 3.3 μM); 2BrIC (at 0.0 μM, 0.120 μM, 0.370 μM, 1.1 μM, or 3.3 μM) and doxorubicin (solid bars); or doxorubicin alone (open bars).

FIG. 14 is a bar graph showing the ability of 6-acetyl-8-cyclopentyl-5-methyl-2-(5-piperazin-1-yl-pyridin-2-ylamino)-8H-pyrido-[2,3-d]pyrimidin-7-one (PD 0332991) to protect human RB-null melanoma cells (A2058) from doxorubicin-induced cytotoxicity as determined by assessing cell viability using the WST-1 assay. Relative cell number was determined by following absorbance at 450 nm. Results are shown for cells treated with either PD 0332991 alone (striped bars; at 0.0 μM, 0.120 μM, 0.370 μM, 1.1 μM, or 3.3 μM); PD 0332991 (at 0.0 μM, 0.120 μM, 0.370 μM, 1.1 μM, or 3.3 μM) and doxorubicin (DOX; solid bars); or DOX alone (open bars).

FIG. 15A is a flow cytometry gating scheme for untreated multipotent progenitor (MPP) cells (top) and 2-bromo-12,13-dihydro-5H-indolo[2,3-a]pyrrolo[3,4]-carbazole-5,6-dione (2BrIC)-treated MPP cells (bottom) using cell surface antigens. In addition to treatment or non-treatment with 2BrIC for 24 hours, cells were also in the presence of 5-bromo-2-deoxyuridine (BrdU).

FIG. 15B is a bar graph showing the percentage of 5-bromo-2-deoxyuridine (BrdU) positive cells in the Lin-Kit+Sca-1 positive untreated and 2-bromo-12,13-dihydro-5H-indolo[2,3-a]pyrrolo[3,4]-carbazole-5,6-dione (2BrIC)-treated cell populations from FIG. 15A. BrdU incorporation is a measure of G1 to S-phase cell cycle traversal, with in vivo 2BrIC treatment clearly reducing proliferation of the MPP.

FIG. 16A is a flow cytometry gating scheme for hematopoietic stem cells (HSC) and multipotent progenitor (MPP) cells (top) and myeloid progenitors (bottom) using cell surface antigens.

FIG. 16B are representative contour plots of proliferation in hematopoietic stem and progenitor cell (HSPC) populations by 5-bromo-2-deoxyuridine (BrdU) incorporation and Ki67 expression after 48 hours of no treatment (N=6) or treatment with 6-acetyl-8-cyclopentyl-5-methyl-2-(5-piperazin-1-yl-pyridin-2-ylamino)-8H-pyrido-[2,3-d]pyrimidin-7-one (PD0332991) and 24 hour exposure to BrdU. Contours represent 5% density. BrdU incorporation in a measure of G1 to S-phase cell cycle traversal and Ki67 expression is a marker of cycling cells. PD 0332991 treatment clearly reduces proliferation in these early HSPC.

FIG. 16C is a set of bar graphs showing the quantification of 5-bromo-2-deoxyuridine (BrdU) and Ki67 data in the untreated (open bars) and treated (shaded bars) cell populations from FIG. 16B. *p,0.05; **p<0.01, ***p<0.001. Error bars show standard error of the mean.

FIG. 16D shows a set of bar graphs showing the relative frequencies of Lin-, HSC, MPP or Lin-cKit+Sca1− populations in the untreated (open bars) and treated (shaded bars) cell populations after 48 hours of treatment and 24 hours of 5-bromo-2-deoxyuridine (BrdU) exposure. *p,0.05; **p<0.01, ***p<0.001. Error bars show standard error of the mean. A relative enrichment of HSC and MPP occurs with cyclin-dependent kinase 4/6 (CDK4/6) inhibitor treatment because more differentiated myeloid cells, which are considerably more abundant, continue to divide and differentiate in the presence of CDK4/6 inhibitor.

FIG. 17 is a set of bar graphs showing that 2-bromo-12,13-dihydro-5H-indolo[2,3-a]pyrrolo[3,4]-carbazole-5,6-dione (2BrIC; 150 mg/kg by oral gavage) provides protection of red blood cells and hemoglobin from the effects of carboplatin (Carbo; 100 mg/kg, i.p.) in vivo in mice. Mice were treated with 2BrIC 1 hour before Carbo injection. Blood was collected on the sixth day following Carbo injection and total blood cell counts were determined. The unshaded bars represent data from animals treated with Carbo and 2BrIC, while the shaded bars represent data from animals treated with Carbo alone. Error bars show standard error of the mean.

FIG. 18 is a set of bar graphs showing that 6-acetyl-8-cyclopentyl-5-methyl-2-(5-piperazin-1-yl-pyridin-2-ylamino)-8H-pyrido-[2,3-d]pyrimidin-7-one (PD 0332991; 150 mg/kg by oral gavage) provides quadrilineage protection from the effects of doxorubicin (DOX; 10 mg/kg, i.p.) in vivo in mice. Mice were treated with PD 0332991 1 hour before DOX injection. DOX injection was repeated after 7 days. Blood was collected fourteen days following initial DOX injection and total blood cell counts were determined. The more lightly shaded bars represent data from animals treated with DOX and PD 0332991, while the more darkly shaded bars represent data from animals treated with DOX alone. Error bars show standard error of the mean.

FIG. 19 is a set of bar graphs showing that 6-acetyl-8-cyclopentyl-5-methyl-2-(5-piperazin-1-yl-pyridin-2-ylamino)-8H-pyrido-[2,3-d]pyrimidin-7-one (PD 0332991; 150 mg/kg by oral gavage) provides quadrilineage protection the effects of from carboplatin (Carbo; 100 mg/kg; i.p.) in vivo in mice. Mice were treated with PD 0332991 one hour before Carbo injection. Blood was collected at seven day intervals and total blood cell counts were determined. The more lightly shaded bars represent data from animals treated with Carbo and PD 0332991, while the more darkly shaded bars represent data from animals treated with Carbo alone. Error bars show standard error of the mean.

FIG. 20A shows flow cytometry gating schemes for various cell types treated with flavopiridol and 5-bromo-2-deoxyuridine (BrdU), showing that flavopiridol does not induce G1 arrest in cyclin-dependent kinase 4/6 (CDK4/6)-dependent cells. The scheme at the top is for human melanoma cells lacking INK4a/ARF (WM2664); the scheme in the middle is for telomerized human diploid fibroblast (tHDF) cells (HS68); and the scheme at the bottom is for human RB-null melanoma cells (A2058).

FIG. 20B is a bar graph showing the absence of chemoprotective effects for flavopiridol in cyclin-dependent kinase 4/6 (CDK4/6)-dependent cells. Data is provided for untreated human melanoma cells lacking INK4a/ARF cells (WM2664; cells); WM2664 cells treated with 900, 300, 100, or 30 nM flavopiridol (16 hours); WM2664 cells treated with Doxorubicin (DOX; 122 nM; 8 hours); and for WM2664 cells treated with DOX (122 nM) for 8 hours following 16 hours of treatment with 900, 300, 100, or 30 nM flavopiridol. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 20C is a bar graph showing the absence of chemoprotective effects for flavopiridol in cyclin-dependent kinase 4/6 (CDK4/6)-dependent cells. Data is provided for untreated telomerized human diploid fibroblast (tHDF) cells (HS68; cells); HS68 cells treated with 900, 300, 100, or 30 nM flavopiridol (16 hours); HS68 cells treated with Doxorubicin (DOX; 370 nM; 8 hours); and for HS68 cells treated with DOX (370 nM) for 8 hours following 16 hour pretreatment with 900, 300, 100, or 30 nM flavopiridol. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 20D is a bar graph showing the absence of chemoprotective effects for flavopiridol in cyclin-dependent kinase 4/6 (CDK4/6)-independent cells. Data is provided for untreated human retinoblastoma tumor suppressor protein (RB)-null melanoma cells (A2058; cells); A2058 cells treated with 900, 300, 100, or 30 nM flavopiridol (16 hours); A2058 cells treated with Doxorubicin (DOX; 370 nM; 8 hours); and for A2058 cells treated with DOX (370 nM) for 8 hours following 16 hour pretreatment with 900, 300, 100, or 30 nM flavopiridol. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 21A shows flow cytometry gating schemes for various cell types treated with compound 7 (R547) and 5-bromo-2-deoxyuridine (BrdU), showing that compound 7 does not induce G1 arrest in cyclin-dependent kinase 4/6 (CDK4/6)-dependent cells. The scheme at the top is for human melanoma cells lacking INK4a/ARF (WM2664); the scheme in the middle is for telomerized human diploid fibroblast (tHDF) cells (HS68); and the scheme at the bottom is for human retinoblastoma tumor suppressor protein (RB)-null melanoma cells (A2058).

FIG. 21B is a bar graph showing the absence of chemoprotective effects for compound 7 (R547) in cyclin-dependent kinase 4/6 (CDK4/6)-dependent cells. Data is provided for untreated human melanoma cells lacking INK4a/ARF cells (WM2664; cells); WM2664 cells treated with 900, 300, 100, or 30 nM compound 7 (16 hours); WM2664 cells treated with Doxorubicin (DOX; 122 nM; 8 hours); and for WM2664 cells treated with DOX (122 nM) for 8 hours following 16 hour pretreatment with 900, 300, 100, or 30 nM compound 7. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 21C is a bar graph showing the absence of chemoprotective effects for compound 7 (R547) in cyclin-dependent kinase 4/6 (CDK4/6)-dependent cells. Data is provided for untreated telomerized human diploid fibroblast (tHDF) cells (H568; cells); HS68 cells treated with 900, 300, 100, or 30 nM compound 7(16 hours); HS68 cells treated with Doxorubicin (DOX; 370 nM; 8 hours); and for HS68 cells treated with DOX (370 nM) for 8 hours following 16 hours pretreatment with 900, 300, 100, or 30 nM compound 7. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 21D is a bar graph showing the absence of chemoprotective effects for compound 7 (R547) in cyclin-dependent kinase 4/6 (CDK4/6)-independent cells. Data is provided for untreated human retinoblastoma tumor suppressor protein (RB)-null melanoma cells (A2058; cells); A2058 cells treated with 900, 300, 100, or 30 nM compound 7 (16 hours); A2058 cells treated with Doxorubicin (DOX; 370 nM; 8 hours); and for A2058 cells treated with DOX (370 nM) for 8 hours following 16 hour pretreatment with 900, 300, 100, or 30 nM compound 7. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 22A shows flow cytometry gating schemes for various cell types treated with Roscovitine and 5-bromo-2-deoxyuridine (BrdU), showing that Roscovitine does not induce G1 arrest in cyclin-dependent kinase 4/6 (CDK4/6)-dependent cells. The scheme at the top is for human melanoma cells lacking INK4a/ARF (WM2664); the scheme in the middle is for telomerized human diploid fibroblast (tHDF) cells (HS68); and the scheme at the bottom is for human RB-null melanoma cells (A2058).

FIG. 22B is a bar graph showing the absence of chemoprotective effects for Roscovitine in cyclin-dependent kinase 4/6 (CDK4/6)-dependent cells. Data is provided for untreated human melanoma cells lacking INK4a/ARF cells (WM2664; cells); WM2664 cells treated with 900, 300, 100, or 30 nM Roscovitine (16 hours); WM2664 cells treated with Doxorubicin (DOX; 122 nM; 8 hours); and for WM2664 cells treated with DOX (122 nM) for 8 hours following 16 hour pretreatment with 900, 300, 100, or 30 nM Roscovitine. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 22C is a bar graph showing the absence of chemoprotective effects for Roscovitine in cyclin-dependent kinase 4/6 (CDK4/6)-dependent cells. Data is provided for untreated telomerized human diploid fibroblast (tHDF) cells (HS68; cells); HS68 cells treated with 900, 300, 100, or 30 nM Roscovitine (16 hours); HS68 cells treated with Doxorubicin (DOX; 370 nM; 8 hours); and for HS68 cells treated with DOX (370 nM) for 8 hours following 16 hour pretreatment with 900, 300, 100, or 30 nM Roscovitine. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 22D is a bar graph showing the absence of chemoprotective effects for Roscovitine in cyclin-dependent kinase 4/6 (CDK4/6)-independent cells. Data is provided for untreated human retinoblastoma tumor suppressor protein (RB)-null melanoma cells (A2058; cells); A2058 cells treated with 900, 300, 100, or 30 nM Roscovitine (16 hours); A2058 cells treated with Doxorubicin (DOX; 370 nM, 8 hours); and for A2058 cells treated with DOX (370 nM) for 8 hours following 16 hour pretreatment with 900, 300, 100, or 30 nM Roscovitine. Treatment media was replaced and cell viability was determined after 7 days using the CellTiter-Glo® assay (CTG; Promega, Madison, Wisconsin, United States of America) and data is presented in relative light units (RLU). Error bars show standard error of the mean.

FIG. 23A is a bar graph showing the absence of chemoprotective effects for Genistein in cyclin-dependent kinase 4/6 (CDK4/6)-dependent cells. Data is provided for untreated human melanoma cells lacking INK4a/ARF cells (WM2664; cells); WM2664 cells treated with 100, 30, 10 or 3 μM Genistein (16 hours); WM2664 cells treated with Doxorubicin (DOX; 122 nM; 8 hours); and for WM2664 cells treated with DOX (122 nM) for 8 hours following 16 hour pretreatment with and 100, 30, 10, or 3 μM Genistein. Treatment media was replaced and cell viability was determined after 7 days using the CellTiter-Glo® assay (CTG; Promega, Madison, Wis., United States of America) and data is presented in relative light units (RLU).

FIG. 23B is a bar graph showing the absence of chemoprotective effects for Genistein in cyclin-dependent kinase 4/6 (CDK4/6)-dependent cells. Data is provided for untreated telomerized human diploid fibroblast (tHDF) cells (HS68; cells); HS68 cells treated with 300, 100, 30, or 3 μM Genistein (16 hours); HS68 cells treated with Doxorubicin (DOX; 370 nM; 8 hours); and for HS68 cells treated with DOX (370 nM) for 8 hours following 16 hour pretreatment with 300, 100, 30, or 3 μM Genistein. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 23C is a bar graph showing the absence of chemoprotective effects for Genistein in cyclin-dependent kinase 4/6 (CDK4/6)-independent cells. Data is provided for untreated human retinoblastoma tumor suppressor protein (RB)-null melanoma cells (A2058; cells); A2058 cells treated with 100, 30, 10, or 3 μM Genistein (16 hours); A2058 cells treated with Doxorubicin (DOX; 370 nM; 8 hours); and for A2058 cells treated with DOX (370 nM) for 8 hours following 16 hour pretreatment with 100, 30, 10, or 3 μM Genistein. Treatment media was replaced and cell viability was determined after 7 days using the CellTiter-Glo® assay (CTG; Promega, Madison, Wis., United States of America) and data is presented in relative light units (RLU). Error bars show standard error of the mean.

FIG. 24A is a bar graph showing the percentage (%) of cyclin-dependent kinase 4/6 (CDK4/6)-dependent cells in the G1 phase following treatment with 1.1 or 3.3 μM of non-CDK4/6 selective compound 8, 9, 11, 14, 10, 13, or 12. For comparison, data is also given for untreated cell populations (controls 1-4).

FIG. 24B is a bar graph showing the percentage (%) of cyclin-dependent kinase 4/6 (CDK4/6)-dependent cells in the G2/M phase following treatment with 1.1 or 3.3 μM of compound 8, 9, 11, 14, 10, 13, or 12. For comparison, data is also given for untreated cell populations (controls 1-4).

FIG. 24C is a bar graph showing the percentage (%) of cyclin-dependent kinase 4/6 (CDK4/6)-dependent cells in the S phase following treatment with 1.1 or 3.3 μM of compound 8, 9, 11, 14, 10, 13, or 12. For comparison, data is also given for untreated cell populations (controls 1-4).

FIG. 24D is a bar graph showing the inability of compound 8 to protect cyclin-dependent kinase 4/6 (CDK4/6)-dependent cells (human melanoma cells lacking INK4a/ARF (WM2664)) from doxorubicin-induced cytotoxicity as determined by assessing cell viability using the WST-1 assay 7 days following cell treatment. Cell number was determined by following absorbance at 450 nm. Results are shown for cells treated with either compound 8 alone for 16 hours (striped bars; at 0.0 μM, 0.120 μM, 0.370 μM, 1.1 μM, or 3.3 μM); compound 8 (at 0.0 μM, 0.120 μM, 0.370 μM, 1.1 μM, or 3.3 μM) for 16 hours followed by doxorubicin (DOX; 122 nM; solid bars) for 8 hours; or DOX alone (122 nM; 8 hours; open bars). Error bars show standard error of the mean.

FIG. 24E is a bar graph showing the inability of compound 9 to protect cyclin-dependent kinase 4/6 (CDK4/6)-dependent cells (human melanoma cells lacking 1NK4a/ARF (WM2664)) from doxorubicin-induced cytotoxicity as determined by assessing cell viability using the WST-1 assay 7 days following cell treatment. Cell number was determined by following absorbance at 450 nm. Results are shown for cells treated with either compound 9 alone for 16 hours (striped bars; at 0.0 μM, 0.120 μM, 0.370 μM, 1.1 μM, or 3.3 μM); compound 9 (at 0.0 μM, 0.120 μM, 0.370 μM, 1.1 μM, or 3.3 μM) for 16 hours followed by 8 hours of doxorubicin (DOX; 122 nM; solid bars); or DOX alone for 8 hours (122 nM; open bars). Error bars show standard error of the mean.

FIG. 24F is a bar graph showing the inability of compound 11 to protect cyclin-dependent kinase 4/6 (CDK4/6)-dependent cells (human melanoma cells lacking INK4a/ARF (WM2664)) from doxorubicin-induced cytotoxicity as determined by assessing cell viability using the WST-1 assay 7 days following cell treatment. Cell number was determined by following absorbance at 450 nm. Results are shown for cells treated with either compound 11 alone for 16 hours (striped bars; at 0.0 μM, 0.120 μM, 0.370 μM, 1.1 μM, or 3.3 μM); compound 11 (at 0.0 μM, 0.120 μM, 0.370 μM, 1.1 μM, or 3.3 μM) for 16 hours followed by 8 hours of treatment with doxorubicin (DOX; 122 nM; solid bars); or DOX alone for 8 hours (122 nM; open bars). Error bars show standard error of the mean.

FIG. 24G is a bar graph showing the inability of compound 8 to protect cyclin-dependent kinase 4/6 (CDK4/6)-dependent cells (telomerized human diploid fibroblast (tHDF) cells (HS68)) from doxorubicin-induced cytotoxicity as determined by assessing cell viability using the WST-1 assay 7 days following cell treatment. Cell number was determined by following absorbance at 450 nm. Results are shown for cells treated with either compound 8 alone for 16 hours (striped bars; at 0.0 μM, 0.120 μM, 0.370 μM, 1.1 μM, or 3.3 μM); compound 8 (at 0.0 μM, 0.120 μM, 0.370 μM, 1.1 μM, or 3.3 μM) for 16 hours followed by 8 hours of treatment with doxorubicin (DOX; 370 nM; solid bars); or DOX alone for 8 hours (370 nM; open bars). Error bars show standard error of the mean.

FIG. 24H is a bar graph showing the inability of compound 9 to protect cyclin-dependent kinase 4/6 (CDK4/6)-dependent cells (telomerized human diploid fibroblast (tHDF) cells (HS68)) from doxorubicin-induced cytotoxicity as determined by assessing cell viability using the WST-1 assay 7 days following cell treatment. Cell number was determined by following absorbance at 450 nm. Results are shown for cells treated with either compound 9 alone for 16 hours (striped bars; at 0.0 μM, 0.120 μM, 0.370 μM, 1.1 μM, or 3.3 μM); compound 9 (at 0.0 μM, 0.120 μM, 0.370 μM, 1.1 μM, or 3.3 μM) for 16 hours followed by 8 hours of treatment with doxorubicin (DOX; 370 nM; solid bars); or DOX alone for 8 hours (370 nM; open bars). Error bars show standard error of the mean.

FIG. 24I is a bar graph showing the inability of compound 11 to protect cyclin-dependent kinase 4/6 (CDK4/6)-dependent cells (telomerized human diploid fibroblast (tHDF) cells (HS68)) from doxorubicin-induced cytotoxicity as determined by assessing cell viability using the WST-1 assay 7 days following cell treatment. Cell number was determined by following absorbance at 450 nm. Results are shown for cells treated with either compound 11 alone for 16 hours (striped bars; at 0.0 μM, 0.120 μM, 0.370 μM, 1.1 μM, or 3.3 μM); compound 11 (at 0.0 μM, 0.120 μM, 0.370 μM, 1.1 μM, or 3.3 μM) for 16 hours followed by 8 hours of treatment with doxorubicin (DOX; 370 nM; solid bars); or DOX alone for 8 hours (370 nM; open bars). Error bars show standard error of the mean.

FIG. 25A is a bar graph showing that 6-acetyl-8-cyclopentyl-5-methyl-2-(5-piperazin-1-yl-pyridin-2-ylamino)-8H-pyrido-[2,3-d]pyrimidin-7-one (PD 0332991) inhibits chemotherapy-induced cytotoxicity in a cyclin-dependent kinase 4/6 (CDK4/6)-dependent manner. Data is provided for untreated human melanoma cells lacking INK4a/ARF cells (WM2664; cells); WM2664 cells incubated with 15 nM, 30 nM, 89 nM or 270 nM PD0332991 for 16 hours; WM2664 cells treated with Carboplatin (Carbo; 50 μM), Doxorubicin (DOX; 122 nM), or Etoposide (Etop; 2.5 μM) for 8 hours; and for WM2664 cells treated with DOX (122 nM), Carbo (50 μM), or Etop (2.5 μM) for 8 hours following 16 hours of treatment with 15 nM, 30 nM, 89 nM or 270 nM PD0332991. Following incubation, an aliquot of culture media was removed and cytotoxicity was assessed by quantifying the amount of adenylate kinase. Data shown are in relative light units (RLU).

FIG. 25B is a bar graph showing that 6-acetyl-8-cyclopentyl-5-methyl-2-(5-piperazin-1-yl-pyridin-2-ylamino)-8H-pyrido-[2,3-d]pyrimidin-7-one (PD 0332991) inhibits chemotherapy-induced cytotoxicity in a cyclin-dependent kinase 4/6 (CDK4/6)-dependent manner. Data is provided for HS68 cells (cells); HS68 cells incubated with 15 nM, 30 nM, 89 nM or 270 nM PD0332991 for 16 hours; HS68 cells treated with Carboplatin (Carbo; 50 μM), Doxorubicin (DOX; 122 nM), or Etoposide (Etop; 2.5 μM) for 8 hours; and for HS68 cells treated with DOX (122 nM), Carbo (50 μM), or Etop (2.5 μM) for 8 hours following 16 hours of treatment with 15 nM, 30 nM, 89 nM or 270 nM PD0332991. Following incubation, an aliquot of culture media was removed and cytotoxicity was assessed by quantifying the amount of adenylate kinase. Data shown are in relative light units (RLU).

FIG. 25C a bar graph showing that 6-acetyl-8-cyclopentyl-5-methyl-2-(5-piperazin-1-yl-pyridin-2-ylamino)-8H-pyrido-[2,3-d]pyrimidin-7-one (PD 0332991) inhibits chemotherapy-induced cytotoxicity in a cyclin-dependent kinase 4/6 (CDK4/6)-dependent manner. Data is provided for untreated retinoblastoma tumor suppressor protein (RB-null)human melanoma cells (A2058; cells); A2058 cells incubated with 15 nM, 30 nM, 89 nM or 270 nM PD0332991 for 16 hours; A2058 cells treated with Carboplatin (Carbo; 50 μM), Doxorubicin (DOX; 122 nM), or Etoposide (Etop; 2.5 μM) for 8 hours; and for A2058 cells treated with DOX (122 nM), Carbo (50 μM), or Etop (2.5 μM) for 8 hours following 16 hours of treatment with 15 nM, 30 nM, 89 nM or 270 nM PD0332991. Following incubation, an aliquot of culture media was removed and cytotoxicity was assessed by quantifying the amount of adenylate kinase. Data shown are in relative light units (RLU).

FIG. 25D is a bar graph showing that staurosporine enhances chemotherapy-induced cytotoxicity in a cyclin-dependent kinase 4/6 (CDK4/6)-independent manner. Data is provided for untreated human melanoma cells lacking INK4a/ARF cells (WM2664; cells); WM2664 cells incubated with 160 pM, 500 pM, 1.5 nM or 4.5 nM staurosporine for 16 hours; WM2664 cells treated with Carboplatin (Carbo; 50 μM), Doxorubicin (DOX; 122 nM), or Etoposide (Etop; 2.5 μM) for 8 hours; and for WM2664 cells treated with DOX (122 nM), Carbo (50 μM), or Etop (2.5 μM) for 8 hours following 16 hours of treatment with 160 pM, 500 pM, 1.5 nM or 4.5 nM staurosporine. Following incubation, an aliquot of culture media was removed and cytotoxicity was assessed by quantifying the amount of adenylate kinase. Data shown are in relative light units (RLU).

FIG. 25E is a bar graph showing that staurosporine enhances chemotherapy-induced cytotoxicity in a cyclin-dependent kinase 4/6 (CDK4/6)-independent manner. Data is provided for HS68 cells (cells); HS68 cells incubated with 160 pM, 500 pM, 1.5 nM or 4.5 nM staurosporine for 16 hours; HS68 cells treated with Carboplatin (Garbo; 50 μM), Doxorubicin (DOX; 122 nM), or Etoposide (Etop; 2.5 μM) for 8 hours; and for HS68 cells treated with DOX (122 nM), Carbo (50 μM), or Etop (2.5 μM) for 8 hours following 24 hours of treatment with 160 pM, 500 pM, 1.5 nM or 4.5 nM staurosporine. Following incubation, an aliquot of culture media was removed and cytotoxicity was assessed by quantifying the amount of adenylate kinase. Data shown are in relative light units (RLU).

FIG. 25F is a is a bar graph showing that staurosporine enhances chemotherapy-induced cytotoxicity in a cyclin-dependent kinase 4/6 (CDK4/6)-independent manner. Data is provided for untreated retinoblastoma tumor suppressor protein (RB-null) human melanoma cells (A2058; cells); A2058 cells incubated with 160 pM, 500 pM, 1.5 nM or 4.5 nM staurosporine for 16 hours; A2058 cells treated with Carboplatin (Carbo; 50 μM), Doxorubicin (DOX; 122 nM), or Etoposide (Etop; 2.5 μM) for 8 hours; and for A2058 cells treated with DOX (122 nM), Carbo (50 μM), or Etop (2.5 μM) for 8 hours following 16 hours of treatment with 160 pM, 500 pM, 1.5 pM or 4.5 pM staurosporine. Following incubation, an aliquot of culture media was removed and cytotoxicity was assessed by quantifying the amount of adenylate kinase. Data shown are in relative light units (RLU).

FIG. 26A is a bar graph showing the percentage (%) of cyclin-dependent kinase 4/6 (CDK4/6)-dependent cells in the G1 (lightly shaded bars), G2/M (darkly shaded bars) and S (unshaded bars) phase following treatment with 160 pM, 500 pM, 1.5 nM or 4.5 nM staurosporine for 24 hours. Staurosporine appears to induce G1 cell cycle arrest in HS68 cells.

FIG. 26B is a bar graph showing the absence of chemoprotective effects for staurosporine in cyclin-dependent kinase 4/6 (CDK4/6)-dependent cells. Data is provided for untreated HS68 cells treated with 160 pM, 500 pM, 1.5 nM 01 4.5 nM staurosporine (16 hours); HS68 cells treated with Doxorubicin (DOX; 122 nM; 8 hours); and for HS68 cells treated with DOX (122 nM) for 8 hours following 16 hour pretreatment with and160 pM, 500 pM, 1.5 nM or 4.5 nM staurosporine (16 hours); HS68 cells treated with carboplatin (Carbo; 50 μM; 8 hours); and for HS68 cells treated with Carbo (50 μM) for 8 hours following 16 hour pretreatment with 160 pM, 500 pM, 1.5 nM or 4.5 nM staurosporine (16 hours); HS68 cells treated with etoposide (Etop; 2.5 μM; 8 hours); and for HS68 cells treated with Etop (2.5 μM) for 8 hours following 16 hour pretreatment with 160 pM, 500 pM, 1.5 nM or 4.5 nM staurosporine (16 hours). Treatment media was replaced and cell viability was determined after 7 days using the CellTiter-Glo® assay (CTG; Promega, Madison, Wis., United States of America) and data is presented in relative light units (RLU). Staurosporine does not appear to protect HS68 cells from chemotherapy-induced cytotoxicity.

FIG. 27A is a bar graph showing the inability of staurosporine to protect cyclin-dependent kinase 4/6 (CDK4/6) dependent cells (human INKa/ARF melanoma cells (WM2664)) from doxorubicin-, carboplatin- or etoposide-induced DNA damage as determined by assessing gamma-H2AX levels. The percentage (%) of gamma-H2AX positive cells is shown for untreated WM2664 cells; for WM2664 cells treated with 160 pM, 500 pM, 1.5 nM, or 4.5 nM staurosporine for 16 hours; for A2058 cells treated with either carboplatin (Carbo, 50 μM), etoposide (Etop, 2.5 μM), or doxorubicin (Dox, 122 nM) alone for 8 hours; and for WM2664 cells treated with either Carbo (50 μM), Etop (2.5 μM) or Dox (122 nM) for 8 hours following pretreatment with 160 pM, 500 pM, 1.5 nM, or 4.5 nM staurosporine for 16 hours. Staurosporine does not appear to protect WM2664 cells from chemotherapy-induced DNA damage.

FIG. 27B is a bar graph showing the inability of staurosporine to protect cyclin-dependent kinase 4/6 (CDK4/6) dependent cells (human telomerized fibroblasts cells (HS68)) from doxorubicin-, carboplatin- or etoposide-induced DNA damage as determined by assessing gamma-H2AX levels. The percentage (%) of gamma-H2AX positive cells is shown for untreated HS68 cells; for HS68 cells treated with 160 pM, 500 pM, 1.5 nM, or 4.5 nM staurosporine for 16 hours; for HS68 cells treated with either carboplatin (Carbo, 50 μM), etoposide (Etop, 2.5 μM), or doxorubicin (Dox, 122 nM) alone for 8 hours; and for HS68 cells treated with either Carbo (50 μM), Etop (2.5 μM), or Dox (122 nM) for 8 hours following pretreatment with 160 pM, 500 pM, 1.5 nM, or 4.5 nM staurosporine for 16 hours. Staurosporine does not appear to protect HS68 cells from hemotherapy-induced DNA damage.

FIG. 27C is a bar graph showing the inability of staurosporine to protect cyclin-dependent kinase 4/6 (CDK4/6) independent cells (human RB-null melanoma cells (A2058)) from doxorubicin-, carboplatin- or etoposide-induced DNA damage as determined by assessing gamma-H2AX levels. The percentage (%) of gamma-H2AX positive cells is shown for untreated A2058 cells; for A2058 cells treated with 160 pM, 500 pM, 1.5 nM, or 4.5 nM staurosporine for 16 hours; for A2058 cells treated with either carboplatin (Carbo, 50 μM), etoposide (Etop, 2.5 μM) or doxorubicin (Dox, 122 nM) alone for 8 hours; and for A2058 cells treated with either Carbo (50 μM), Etop (2.5 μM), or Dox (122 nM) for 8 hours following pretreatment with 160 pM, 500 pM, 1.5 nM, or 4.5 nM staurosporine for 16 hours. Staurosporine does not appear to protect A2058 cells from chemotherapy-induced DNA damage.

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all active optical and stereoisomers, as well as racemic mixtures where such isomers and mixtures exist.

### I. Definitions

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a compound” or “a cell” includes a plurality of such compounds or cells, and so forth.

The term “and/or” when used in describing two items or conditions, e.g., CDK4 and/or CDK6, refers to situations where both items or conditions are present or applicable and to situations wherein only one of the items or conditions is present or applicable. Thus, a CDK4 and/or CDK6 inhibitor can be a compound that inhibits both CDK4 and CDK6, a compound that inhibits only CDK4, or a compound that only inhibits CDK6.

By “healthy cell” or “normal cell” is meant any cell in a subject that does not display the symptoms or markers of a disease (e.g., cancer or another proliferative disease). In some embodiments, the healthy cell is a stem cell. In some embodiments, the healthy cell is a hematopoietic stem or progenitor cell. Progenitor cells include, but are not limited to, long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

The term “cancer” as used herein refers to diseases caused by uncontrolled cell division and the ability of cells to metastasize, or to establish new growth in additional sites. The terms “malignancy”, “neoplasm”, “tumor” and variations thereof refer to cancerous cells or groups of cancerous cells.

Specific types of cancer include, but are not limited to, skin cancers, connective tissue cancers, adipose cancers, breast cancers, lung cancers, stomach cancers, pancreatic cancers, ovarian cancers, cervical cancers, uterine cancers, anogenital cancers, kidney cancers, bladder cancers, colon cancers, prostate cancers, head and neck cancers, brain cancers, central nervous system (CNS) cancers, retinal cancer, blood, and lymphoid cancers.

As used herein the term “chemotherapy” refers to treatment with a cytotoxic compound (e.g., a DNA damaging compound) to reduce or eliminate the growth or proliferation of undesirable cells, such as, but not limited to, cancer cells. Thus, as used herein, “chemotherapeutic compound” refers to a cytotoxic compound used to treat cancer. The cytotoxic effect of compound can be the result of one or more of nucleic acid intercalation or binding, DNA or RNA alkylation, inhibition of RNA or DNA synthesis, the inhibition of another nucleic acid-related activity (e.g., protein synthesis), or any other cytotoxic effect.

Thus, a “cytotoxic compound” can be any one or any combination of compounds also described as “antineoplastic” agents or “chemotherapeutic agents.” Such compounds include, but are not limited to, DNA damaging compounds and other chemicals that can kill cells. “DNA damaging compounds” include, but are not limited to, alkylating agents, DNA intercalators, protein synthesis inhibitors, inhibitors of DNA or RNA synthesis, DNA base analogs, topoisomerase inhibitors, and telomerase inhibitors or telomeric DNA binding compounds. For example, alkylating agents include alkyl sulfonates, such as busulfan, improsulfan, and piposulfan; aziridines, such as a benzodizepa, carboquone, meturedepa, and uredepa; ethylenimines and methylmelamines, such as altretamine, triethylenemelamine, triethylenephosphoramide, triethylenethiophosphoramide, and trimethylolmelamine; nitrogen mustards such as chlorambucil, chlornaphazine, cyclophosphamide, estramustine, iphosphamide, mechlorethamine, mechlorethamine oxide hydrochloride, melphalan, novembichine, phenesterine, prednimustine, trofosfamide, and uracil mustard; and nitroso ureas, such as carmustine, chlorozotocin, fotemustine, lomustine, nimustine, and ranimustine.

Antibiotics used in the treatment of cancer include dactinomycin, daunorubicin, doxorubicin, idarubicin, bleomycin sulfate, mytomycin, plicamycin, and streptozocin. Chemotherapeutic antimetabolites include mercaptopurine, thioguanine, cladribine, fludarabine phosphate, fluorouracil (5-FU), floxuridine, cytarabine, pentostatin, methotrexate, and azathioprine, acyclovir, adenine β-1-D-arabinoside, amethopterin, aminopterin, 2-aminopurine, aphidicolin, 8-azaguanine, azaserine, 6-azauracil, 2′-azido-2′-deoxynucleosides, 5-bromodeoxycytidine, cytosine β-1-D-arabinoside, diazooxynorleucine, dideoxynucleosides, 5-fluorodeoxycytidine, 5-fluorodeoxyuridine, and hydroxyurea.

Chemotherapeutic protein synthesis inhibitors include abrin, aurintricarboxylic acid, chloramphenicol, colicin E3, cycloheximide, diphtheria toxin, edeine A, emetine, erythromycin, ethionine, fluoride, 5-fluorotryptophan, fusidic acid, guanylyl methylene diphosphonate and guanylyl imidodiphosphate, kanamycin, kasugamycin, kirromycin, and O-methyl threonine. Additional protein synthesis inhibitors include modeccin, neomycin, norvaline, pactamycin, paromomycine, puromycin, ricin, shiga toxin, showdomycin, sparsomycin, spectinomycin, streptomycin, tetracycline, thiostrepton, and trimethoprim.

Inhibitors of DNA synthesis, include alkylating agents such as dimethyl sulfate, mitomycin C, nitrogen and sulfur mustards; intercalating agents, such as acridine dyes, actinomycins, adriamycin, anthracenes, benzopyrene, ethidium bromide, propidium diiodide-intertwining; and other agents, such as distamycin and netropsin. Topoisomerase inhibitors, such as coumermycin, nalidixic acid, novobiocin, and oxolinic acid; inhibitors of cell division, including colcemide, colchicine, vinblastine, and vincristine; and RNA synthesis inhibitors including actinomycin D, α-amanitine and other fungal amatoxins, cordycepin (3′-deoxyadenosine), dichlororibofuranosyl benzimidazole, rifampicine, streptovaricin, and streptolydigin also can be used as the DNA damaging compound

Thus, current chemotherapeutic compounds whose toxic effects can be mitigated by the presently disclosed selective CDK4/6 inhibitors include, adrimycin, 5-fluorouracil (5FU), etoposide, camptothecin, actinomycin-D, mitomycin, cisplatin, hydrogen peroxide, carboplatin, procarbazine, mechlorethamine, cyclophosphamide, ifosfamide, melphalan, chlorambucil, bisulfan, nitrosurea, dactinomycin, daunorubicin, doxorubicin, bleomycin, plicomycin, tamoxifen, taxol, transplatinum, vinblastin, and methotrexate, and the like.

By “at risk of incurring exposure to a cytotoxic compound” is meant a subject scheduled for (such as by scheduled chemotherapy sessions) exposure to cytotoxic (e.g., DNA damaging) agents in the future or a subject having a chance of being exposed to a cytotoxic compound inadvertently in the future. Inadvertent exposure includes accidental or unplanned environmental or occupational exposure or to overdose with a cytotoxic compound incurred as part of a medical treatment.

By “effective amount of an inhibitor compound” is meant an amount effective to reduce or eliminate the toxicity associated with chemotherapy or other exposure to a cytotoxic compound in healthy HSPCs in the subject. In some embodiments, the effective amount is the amount required to temporarily (e.g., for a few hours or days) inhibit the proliferation of hematopoietic stem cells (i.e., to induce a quiescent state in hematopoietic stem cells) in the subject.

By “long-term hematological toxicity” is meant hematological toxicity affecting a subject for a period lasting more than one or more weeks, months or years following administration of the cytotoxic compound. Long-term hematological toxicity can result in bone marrow disorders that can cause the ineffective production of blood cells (i.e., myelodysplasia) and/or lymphocytes. Hematological toxicity can be observed, for example, as anemia, reduction in platelet count (i.e., thrombocytopenia) or reduction in white blood cell count (i.e., neutropenia). In some cases, myelodysplasia can result in the development of leukemia. Long-term toxicity related to chemotherapy can also damage other self renewing cells in a subject, in addition to hematological cells. Thus, long-term toxicity can also lead to graying and frailty.

By “free of” is meant that subjects treated with a selective CDK4/6 inhibitor by the presently disclosed methods do not display any detectable signs or symptoms of long-term hematologic toxicity or display signs or symptoms of long-term hematologic toxicity that are significantly reduced (e.g., reduced 10 times, or reduced 100 times or more) compared to the signs/symptoms that would be displayed by subjects treated with the cytotoxic compound who did not receive a dose or doses of a CDK4/6 inhibitor.

“Free of” can also refer to a selective CDK4/6 inhibitor compound not having an undesired or off-target effect, particularly when used in vivo or assessed via a cell-based assay. Thus, “free of” can refer to a selective CDK4/6 inhibitor not having off-target effects such as, but not limited to, long term toxicity, anti-oxidant effects, estrogenic effects, tyrosine kinase inhibitory effects, inhibitory effects on CDKs other than CDK4/6; and cell cycle arrest in CDK4/6-independent cells.

A CDK4/6 inhibitor that is “substantially free” of off-target effects is a CDK4/6 inhibitor that can have some minor off-target effects that do not interfere with the inhibitor's ability to provide protection from cytotoxic compounds in CDK4/6-dependent cells. For example, a CDK4/6 inhibitor that is “substantially free” of off-target effects can have some minor inhibitory effects on other CDKs (e.g., IC₅₀s for CDK1 or CDK2 that are >0.5 μM; >1.0 μM, or >5.0 μM), so long as the inhibitor provides selective G1 arrest in CDK4/6-dependent cells.

By “reduced” and “prevented” or grammatical variations thereof mean, respectively, lessening the undesirable side effects of a medical treatment or keeping the undesirable side effects from occurring completely.

In some embodiments, the subject treated in the presently disclosed subject matter is desirably a human subject, although it is to be understood the methods described herein are effective with respect to all vertebrate species, which are intended to be included in the term “subject.”

More particularly, provided herein is the treatment of mammals, such as humans, as well as those mammals of importance due to being endangered (such as Siberian tigers), of economical importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Thus, embodiments of the methods described herein include the treatment of livestock, including, but not limited to, domesticated swine (pigs and hogs), ruminants, horses, poultry, and the like.

As used herein the term “alkyl” refers to C₁₋₂₀ inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and alkenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “aryl” is used herein to refer to an aromatic moiety that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds. The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, carbonyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyran, pyridine, imidazole, benzimidazole, isothiazole, isoxazole, pyrazole, pyrazine, triazine, pyrimidine, quinoline, isoquinoline, indole, carbazole, and the like.

The term “heteroaryl” refers to aryl groups wherein at least one atom of the backbone of the aromatic ring or rings is an atom other than carbon. Thus, heteroaryl groups have one or more non-carbon atoms selected from the group including, but not limited to, nitrogen, oxygen, and sulfur.

As used herein, the term “acyl” refers to an organic carboxylic acid group wherein the —OH of the carboxyl group has been replaced with another substituent (i.e., as represented by RCO—, wherein R is an alkyl or an aryl group as defined herein). As such, the term “acyl” specifically includes arylacyl groups, such as an acetylfuran and a phenacyl group. Specific examples of acyl groups include acetyl and benzoyl.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl.

The terms “heterocycle” or “heterocyclic” refer to cycloalkyl groups (i.e., non-aromatic, cyclic groups as described hereinabove) wherein one or more of the backbone carbon atoms of a cyclic ring is replaced by a heteroatom (e.g., nitrogen, sulfur, or oxygen). Examples of heterocycles include, but are not limited to, tetrahydrofuran, tetrahydropyran, morpholine, dioxane, piperidine, piperazine, and pyrrolidine.

“Alkoxyl” or “alkoxy” refers to an alkyl-O— group wherein alkyl is as previously described. The term “alkoxyl” as used herein can refer to, for example, methoxyl, ethoxyl, propoxyl, isopropoxyl, butoxyl, t-butoxyl, and pentoxyl. The term “oxyalkyl” can be used interchangably with “alkoxyl”.

“Aryloxyl” or “aryloxy” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl. “Aralkyl” refers to an aryl-alkyl- group wherein aryl and alkyl are as previously described, and included substituted aryl and substituted alkyl. Exemplary aralkyl groups include benzyl, phenylethyl, and naphthylmethyl.

“Aralkyloxyl” or “aralkyloxy” refers to an aralkyl-O— group wherein the aralkyl group is as previously described. An exemplary aralkyloxyl group is benzyloxyl.

The term “amino” refers to the —NR′R″ group, wherein R′ and R″ are each independently selected from the group including H and substituted and unsubstituted alkyl, cycloalkyl, heterocycle, aralkyl, aryl, and heteroaryl. In some embodiments, the amino group is —NH₂. “Aminoalkyl” and “aminoaryl” refer to the —NR′R″ group, wherein R′ is as defined hereinabove for amino and R″ is substituted or unsubstituted alkyl or aryl, resectively.

“Acylamino” refers to an acyl-NH— group wherein acyl is as previously described.

The term “carbonyl” refers to the —(C═O)— or a double bonded oxygen substituent attached to a carbon atom of a previously named parent group.

The term “carboxyl” refers to the —COOH group.

The terms “halo”, “halide”, or “halogen” as used herein refer to fluoro, chloro, bromo, and iodo groups.

The terms “hydroxyl” and “hydroxy” refer to the —OH group.

The term “oxo” refers to a compound described previously herein wherein a carbon atom is replaced by an oxygen atom.

The term “cyano” refers to the —CN group.

The term “nitro” refers to the —NO₂ group.

The term “thio” refers to a compound described previously herein wherein a carbon or oxygen atom is replaced by a sulfur atom.

### II. Hematopoietic Stem and Progenitor Cells and Cyclin-Dependent Kinase Inhibitors

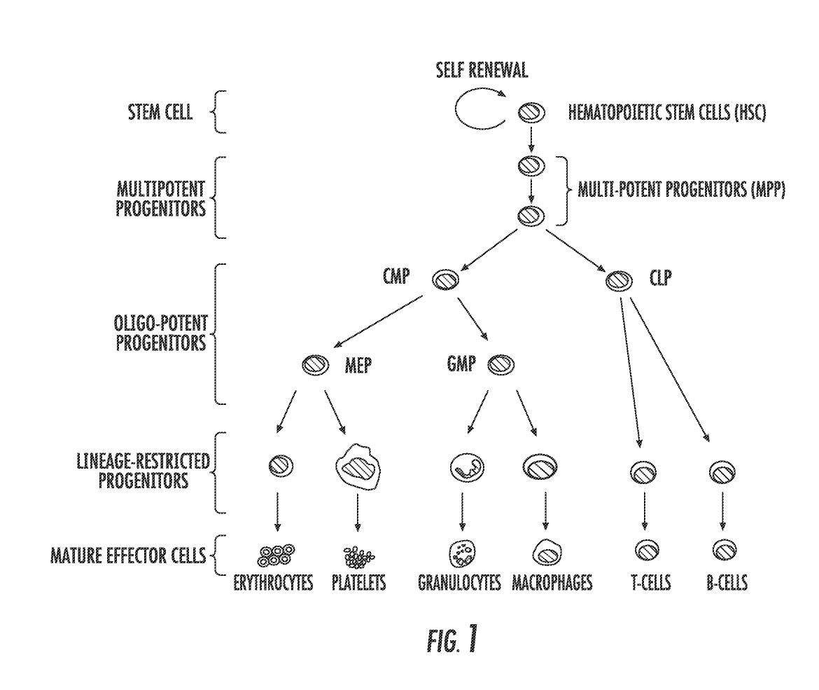

Tissue-specific stem cells are capable of self-renewal, meaning that they are capable of replacing themselves throughout the adult mammalian lifespan through regulated replication. Additionally, stem cells divide asymmetrically to produce “progeny” or “progenitor” cells that in turn produce various components of a given organ. For example, in the hematopoietic system, the hematopoietic stem cells give rise to progenitor cells which in turn give rise to all the differentiated components of blood (e.g., white blood cells, red blood cells, lymphocytes and platelets). See FIG. 1.

The presently disclosed subject matter relates to the specific biochemical requirements of early hematopoietic stem/progenitor cells (HSPC) in the adult mammal. In particular, it has been found that these cells require the enzymatic activity of the proliferative kinases cyclin-dependent kinase 4 (CDK4) and/or cyclin-dependent kinase 6 (CDK6) for cellular replication. In contrast, the vast majority of proliferating cells in adult mammals do not require the activity of CDK4 and/or CDK6 (i.e., CDK4/6). These differentiated cells can proliferate in the absence of CDK4/6 activity by using other proliferative kinases, such as cyclin-dependent kinase 2 (CDK2) or cyclin-dependent kinase 1 (CDK1). Therefore, it is believed that treatment of mammals with a selective CDK4/6 inhibitor can lead to inhibition of proliferation (i.e., pharmacologic quiescence (PQ)) in very restricted stem and progenitor compartments.

Many of the most acute and severe toxicities of chemotherapy are through effects on stem and progenitor cells. Thus, making HSPCs chemoresistant can protect the entire organism from the acute and chronic toxicities of chemotherapy. The presently disclosed subject matter relates to methods of protecting HSPCs in a subject from the toxicity of cytotoxic (e.g., DNA damaging) compounds by the administration of selective CDK4/6 inhibitors. Without being bound to any one theory, administration of such inhibitors is expected to force stem and progenitor cells in the subject into PQ, so that the HSPCs are more resistant to the cytotoxic effect of the chemotherapeutic compound than proliferating cells.

Accordingly, the presently disclosed subject matter provides, in some embodiments, a method of protecting mammals from the acute and chronic toxic effects of chemotherapeutic compounds by forcing hematopoietic stem and progenitor cells (HSPCs) into a quiescent state by transient (e.g., over a less than 48, 24, 20, 16, 12, 10, 8, 6, 4, 2, or 1 hour period) treatment with an non-toxic, selective CDK4/6 inhibitor (e.g., an orally available, non-toxic CDK4/6 inhibitor). During the period of quiescence, the subject's HSPC are more resistant to certain effects of the chemotherapeutic compound. The HSPCs recover from this period of transient quiescence, and then function normally after treatment with the inhibitor is stopped. Thus, chemoprotection with selective CDK4/6 inhibitors can provide marked bone marrow protection and can lead to a more rapid recovery of peripheral blood cell counts (hematocrit, platelets, lymphocytes, and myeloid cells) after chemotherapy.

U.S. Pat. No. 6,369,086 to Davis et al. (hereinafter “the '086 Patent”) appears to describe that selective CDK inhibitors can be useful in limiting the toxicity of cytotoxic agents and can be used to protect from chemotherapy-induced alopecia. In particular, the '086 Patent describes oxindole compounds as specific CDK2 inhibitors. A related journal reference (see Davis et al., Science, 291, 134-137 (2001)) appears to describe that the inhibition of CDK2 produces cell cycle arrest, reducing the sensitivity of the epithelium to cell cycle-active antitumor agents and can prevent chemotherapy-induced alopecia. However, this journal reference was later retracted due to the irreproducibility of the results. In contrast to these purported protective effects of selective CDK2 inhibitors, for which a question is raised by the retraction of the journal article, the presently disclosed subject matter relates to protection of HSPCs and protection from hematological toxicity.

The ability to protect stem/progenitor cells is desirable both in the treatment of cancer and in mitigating the effects of accidental exposure to or overdose with cytotoxic chemicals. The protective effects of the selective CDK4/6 inhibitors can be provided to the subject via pretreatment with the inhibitor (i.e., prior CDK4/6 inhibitor treatment of a subject scheduled to be treated with or at risk of exposure to a cytotoxic compound), concomitant treatment with the CDK4/6 inhibitor and cytotoxic compound, or post-treatment with the CDK4/6 inhibitor (i.e., treatment with the CDK4/6 inhibitor following exposure to the cytotoxic compound). Thus, in some embodiments, the presently disclosed methods relates to the use of selective CDK4/6 inhibitory compounds to provide chemoprotection to subjects undergoing or about to undergo treatment with chemotherapeutic compounds, and to protect subjects from other exposure to cytotoxic compounds.

As used herein the term “selective CDK4/6 inhibitor compound” refers to a compound that selectively inhibits at least one of CDK4 and CDK6 or whose predominant mode of action is through inhibition of CDK4 and/or CDK 6. Thus, selective CDK4/6 inhibitors are compounds that generally have a lower 50% inhibitory concentration (IC₅₀) for CDK4 and/or CDK6 than for other kinases. In some embodiments, the selective CDK4/6 inhibitor can have an IC₅₀ for CDK4 or CDK6 that is at least 2, 3, 4, 5, 6, 7, 8, 9, or 10 times lower than the compound's IC₅₀s for other CDKs (e.g., CDK1 and CDK2). In some embodiments, the selective CDK4/6 inhibitor can have an IC₅₀ for CDK4 or CDK6 that is at least 20, 30, 40, 50, 60, 70, 80, 90, or 100 times lower than the compound's IC₅₀s for other CDKs. In some embodiments, the selective CDK4/6 inhibitor can have an IC₅₀ that is more than 100 times or more than 1000 times less than the compound's IC₅₀s for other CDKs. In some embodiments, the selective CDK4/6 inhibitor compound is a compound that selectively inhibits both CDK4 and CDK6.

In some embodiments, the selective CDK4/6 inhibitor compound is a compound that selectively induces G1 cell cycle arrest in CDK4/6 dependent cells. Thus, when treated with the selective CDK4/6 inhibitor compound according to the presently disclosed methods, the percentage of CDK4/6-dependent cells in the G1 phase increase, while the percentage of CDK4/6-dependent cells in the 02/M phase and S phase decrease. In some embodiments, the selective CDK4/6 inhibitor is a compound that induces substantially pure (i.e., “clean”) G1 cell cycle arrest in the CDK4/6-dependent cells (e.g., wherein treatment with the selective CDK4/6 inhibitor induces cell cycle arrest such that the majority of cells are arrested in G1 as defined by standard methods (e.g., propidium iodide staining or others) and with the population of cells in the G2/M and S phases combined being 20%, 15%, 12%, 10%, 8%, 6%, 5%, 4%, 3%, 2%, 1% or less of the total cell population).

While staurosporine, a non-specific kinase inhibitor, has been reported to indirectly induce G1 arrest in some cell types (see Chen et al., J. Nat. Cancer Inst., 92, 1999-2008 (2000)), the presently disclosed use of selective CDK4/6 inhibitors to directly and selectively induce G1 cell cycle arrest in cells, such as specific fractions of HSPCs, can provide chemoprotection with reduced long term toxicity and without the need for prolonged (e.g., 48 hour or longer) treatment with the inhibitor prior to exposure with the DNA damaging compound. In particular, while some nonselective kinase inhibitors can cause G1 arrest in some cell types by decreasing CDK4 protein levels, benefits of the presently disclosed methods are, without being bound to any one theory, believed to be due at least in part to the ability of selective CDK4/6 inhibitors to directly inhibit the kinase activity of CDK4/6 in HSPCs without decreasing their cellular concentration.

In some embodiments, the selective CDK4/6 inhibitor compound is a compound that is substantially free of off target effects, particularly related to inhibition of kinases other than CDK4 and or CDK6. In some embodiments, the selective CDK4/6 inhibitor compound is a poor inhibitor (e.g., >1 μM IC₅₀) of CDKs other than CDK4/6 (e.g., CDK 1 and CDK2). In some embodiments, the selective CDK4/6 inhibitor compound does not induce cell cycle arrest in CDK4/6-independent cells. In some embodiments, the selective CDK4/6 inhibitor compound is a poor inhibitor (e.g., >1 μM IC₅₀) of tyrosine kinases. Additional, undesirable off-target effects include, but are not limited to, long term toxicity, anti-oxidant effects, and estrogenic effects.

Anti-oxidant effects can be determined by standard assays known in the art. For example, a compound with no significant anti-oxidant effects is a compound that does not significantly scavenge free-radicals, such as oxygen radicals. The anti-oxidant effects of a compound can be compared to a compound with known anti-oxidant activity, such as genistein. Thus, a compound with no significant anti-oxidant activity can be one that has less than about 2, 3, 5, 10, 30, or 100 fold anti-oxidant activity relative to genistein. Estrogenic activities can also be determined via known assays. For instance, a non estrogenic compound is one that does not significantly bind and activate the estrogen receptor. A compound that is substantially free of estrogenic effects can be one that has less than about 2, 3, 5, 10, 20, or 100 fold estrogenic activity relative to a compound with estrogenic activity, e.g., genistein.

Selective CDK4/6 inhibitors that can be used according to the presently disclosed methods include any known small molecule (e.g., <1000 Daltons, <750 Daltons, or less than <500 Daltons), selective CDK4/6 inhibitor, or pharmaceutically acceptable salt thereof. In some embodiments, the inhibitor is a non-naturally occurring compound (i.e., a compound not found in nature). Several classes of chemical compounds have been reported as having CDK4/6 inhibitory ability (e.g., in cell free assays). Selective CDK4/6 inhibitors useful in the presently disclosed methods can include, but are not limited to, pyrido[2,3-d]pyrimidines (e.g., pyrido[2,3-d]pyrimidin-7-ones and 2-amino-6-cyano-pyrido[2,3-d]pyrimidin-4-ones), triaminopyrimidines, aryl[a]pyrrolo[3,4-d]carbazoles, nitrogen-containing heteroaryl-substituted ureas, 5-pyrimidinyl-2-aminothiazoles, benzothiadiazines, acridinethiones, and isoquinolones.

In some embodiments, the pyrido[2,3-d]pyrimidine is a pyrido[2,3-d]pyrimidinone. In some embodiments the pyrido[2,3-d]pyrimidinone is pyrido[2,3-d]pyrimidin-7-one. In some embodiments, the pyrido[2,3-d]pyrimidin-7-one is substituted by an aminoaryl or aminoheteroaryl group. In some embodiments, the pyrido[2,3-d]pyrimidin-7-one is substituted by an aminopyridine group. In some embodiments, the pyrido[2,3-d]pyrimidin-7-one is a 2-(2-pyridinyl)amino pyrido[2,3-d]pyrimidin-7-one. For example, the pyrido[2,3-d]pyrimidin-7-one compound can have a structure of Formula (II) as described in U.S. Patent Publication No. 2007/0179118 to Barvian et al., herein incorporated by reference in its entirety. In some embodiments, the pyrido[2,3-d]pyrimidine compound is 6-acetyl-8-cyclopentyl-5-methyl-2-(5-piperazin-1-yl-pyridin-2-ylamino)-8H-pyrido-[2,3-d]pyrimidin-7-one (i.e., PD 0332991) or a pharmaceutically acceptable salt thereof. See Toogood et al., J. Med. Chem., 2005, 48, 2388-2406.

In some embodiments, the pyrido[2,3-d]pyrimidinone is a 2-amino-6-cyano-pyrido[2,3-d]pyrimidin-4-ones. Selective CDK4/6 inhibitors comprising a 2-amino-6-cyano-pyrido[2,3-d]pyrimidin-4-one are described, for example, by Tu et al. See Tu et al., Bioorg. Med. Chem. Lett., 2006, 16, 3578-3581.

As used herein, “triaminopyrimidines” are pyrimidine compounds wherein at least three carbons in the pyrimidine ring are substituted by groups having the formula —NR₁R₂, wherein R₁ and R₂ are independently selected from the group consisting of H, alkyl, aralkyl, cycloalkyl, heterocycle, aryl, and heteroaryl. Each R₁ and R₂ alkyl, aralkyl, cycloalkyl, heterocycle, aryl, and heteroaryl groups can further be substituted by one or more hydroxyl, halo, amino, alkyl, aralkyl, cycloalkyl, heterocyclic, aryl, or heteroaryl groups. In some embodiments, at least one of the amino groups is an alkylamino group having the structure —NHR, wherein R is C₁-C₆ alkyl. In some embodiments, at least one amino group is a cycloalkylamino group or a hydroxyl-substituted cycloalkylamino group having the formula —NHR wherein R is C₃-C₇ cycloalkyl, substituted or unsubstituted by a hydroxyl group. In some embodiments, at least one amino group is a heteroaryl-substituted aminoalkyl group, wherein the heteroaryl group can be further substituted with an aryl group substituent.

Aryl[a]pyrrolo[3,4-d]carbazoles include, but are not limited to napthyl[a]pyrrolo[3,4-c]carbazoles, indolo[a]pyrrolo[3,4-c]carbazoles, quinolinyl[a]pyrrolo[3,4-c]carbazoles, and isoquinolinyl[a]pyrrolo[3,4-c]carbazoles. See e.g., Engler et al., Bioorg. Med. Chem. Lett., 2003, 13, 2261-2267; Sanchez-Martinez et al., Bioorg. Med. Chem. Lett., 2003, 13, 3835-3839; Sanchez-Martinez et al., Bioorg. Med. Chem. Lett., 2003, 13, 3841-3846; Zhu et al., Bioorg. Med. Chem. Lett., 2003, 13, 1231-1235; and Zhu et al., J. Med Chem., 2003, 46, 2027-2030. Suitable aryl[a]pyrrolo[3,4-d]carbazoles are also disclosed in U.S. Patent Publication Nos. 2003/0229026 and 2004/0048915.

Nitrogen-containing heteroaryl-substituted ureas are compounds comprising a urea moiety wherein one of the urea nitrogen atoms is substituted by a nitrogen-containing heteraryl group. Nitrogen-containing heteroaryl groups include, but are not limited to, five to ten membered aryl groups including at least one nitrogen atom. Thus, nitrogen-containing heteroaryl groups include, for example, pyridine, pyrrole, indole, carbazole, imidazole, thiazole, isoxazole, pyrazole, isothiazole, pyrazine, triazole, tetrazole, pyrimidine, pyridazine, purine, quinoline, isoquinoline, quinoxaline, cinnoline, quinazoline, benzimidazole, phthalimide and the like. In some embodiments, the nitrogen-containing heteroaryl group can be substituted by one or more alkyl, cycloalkyl, heterocyclic, aralkyl, aryl, heteroaryl, hydroxyl, halo, carbonyl, carboxyl, nitro, cyano, alkoxyl, or amino group. In some embodiments, the nitrogen-containing heteroaryl substituted urea is a pyrazole-3-yl urea. The pyrazole can be further substituted by a cycloalkyl or heterocyclic group. In some embodiments, the pyrazol-3-yl urea is:

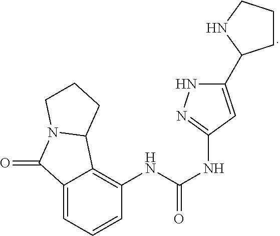

See Ikuta, et al., J. Biol. Chem., 2001, 276, 27548-27554. Additional ureas that can be used according to the presently disclosed subject matter include the biaryl urea compounds of Formula (I) described in U.S. Patent Publication No. 2007/0027147. See also, Honma et al., J. Med. Chem., 2001, 44, 4615-4627; and Honma et al., J. Med. Chem., 2001, 44, 4628-4640.

Suitable 5-pyrimidinyl-2-aminothiazole CDK4/6 inhibitors are described by Shimamura et al. See Shimamura et al., Bioorg. Med. Chem. Lett., 2006, 16, 3751-3754. In some embodiments, the 5-pyrimidinyl-2-aminothiazole has the structure:

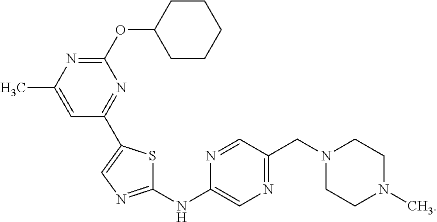

Useful benzothiadiazine and acridinethiones compounds include those, for example, disclosed by Kubo et al. See Kubo et al., Clin. Cancer Res. 1999, 5, 4279-4286 and in U.S. Patent Publication No. 2004/0006074, herein incorporated by reference in their entirety. In some embodiments, the benzothiadiazine is substituted by one or more halo, haloaryl, or alkyl group. In some embodiments, the benzothiadiazine is selected from the group consisting of 4-(4-fluorobenzylamino)-1,2,3-benzothiadiazine-1,1-dioxide, 3-chloro-4-methyl-4H-benzo[e][1,2,4]thiadiazine-1,1-dioxide, and 3-chloro-4-ethyl-4H-benzo[e][1,2,4]thiadiazine-1,1-dioxide. In some embodiments, the acridinethione is substituted by one or more amino or alkoxy group. In some embodiments, the acridinethione is selected from the group consisting of 3-amino-10H-acridone-9-thione (3ATA), 9(10H)-acridinethione, 1,4-dimethoxy-10H-acridine-9-thione, and 2,2′-diphenyldiamine-bis-[N,N′-[3-amido-N-methylamino)-10H-acridine-9-thione]].

In some embodiments, the subject of the presently disclosed methods will be a subject who has been exposed to, is being exposed to, or is scheduled to be exposed to, a chemotherapeutic compound while undergoing therapeutic treatment for a proliferative disorder. Such disorders include cancerous and non-cancer proliferative diseases. For example, the presently disclosed compounds are believed effective in protecting healthy HSPCs during chemotherapeutic treatment of a broad range of tumor types, including but not limited to the following: breast, prostate, ovarian, skin, lung, colorectal, brain (i.e., glioma) and renal.

Ideally, growth of the cancer being treated by the chemotherapeutic compound should not be affected by the selective CDK4/6 inhibitor, as it is preferable that the selective CDK4/6 inhibitor not compromise the efficacy of the chemotherapeutic compound by itself arresting the growth of the cancer cells. Most cancers appear not to depend on the activities of CDK4/6 for proliferation as they can use the proliferative kinases promiscuously (e.g., can use CDK 1/2/4/ or 6) or lack the function of the retinoblastoma tumor suppressor protein (RB), which is inactivated by the CDKs. Therefore, isolated inhibition of CDK4/6 should not affect the chemotherapy response in the majority of cancers. As would be understood by one of skill in the art, the potential sensitivity of certain tumors to CDK4/6 inhibition can be deduced based on tumor type and molecular genetics. Cancers that are not expected to be affected by the inhibition of CDK4/6 are those that can be characterized by one or more of the group including, but not limited to, increased activity of CDK1 or CDK2, loss or absence of retinoblastoma tumor suppressor protein (RB), high levels of MYC expression, increased cyclin E and increased cyclin A. Such cancers can include, but are not limited to, small cell lung cancer, retinoblastoma, HPV positive malignancies like cervical cancer and certain head and neck cancers, MYC amplified tumors such as Burkitts Lymphoma, and triple negative breast cancer; certain classes of sarcoma, certain classes of non-small cell lung carcinoma, certain classes of melanoma, certain classes of pancreatic cancer, certain classes of leukemia, certain classes of lymphoma, certain classes of brain cancer, certain classes of colon cancer, certain classes of prostate cancer, certain classes of ovarian cancer, certain classes of uterine cancer, certain classes of thyroid and other endocrine tissue cancers, certain classes of salivary cancers, certain classes of thymic carcinomas, certain classes of kidney cancers, certain classes of bladder cancer and certain classes of testicular cancers.

For example, in some embodiments, the cancer is selected from a small cell lung cancer, retinoblastoma and triple negative (ER/PR/Her2 negative) or “basal-like” breast cancer. Small cell lung cancer and retinoblastoma almost always inactivate the retinoblastoma tumor suppressor protein (RB), and therefore does not require CDK4/6 activity to proliferate. Thus, CDK4/6 inhibitor treatment will effect PQ in the bone marrow and other normal host cells, but not in the tumor. Triple negative (basal-like) breast cancer is also almost always RB-null. Also, certain virally induced cancers (e.g. cervical cancer and subsets of Head and Neck cancer) express a viral protein (E7) which inactivates RB making these tumors functionally RB-null. Some lung cancers are also believed to be caused by HPV. As would be understood by one of skill in the art, cancers that are not expected to be affected by CDK4/6 inhibitors (e.g., those that are RB-null, that express viral protein E7, or that overexpress MYC) can be determined through methods including, but not limited to, DNA analysis, immunostaining, Western blot analysis, and gene expression profiling.

In part, the effects of chemoprotective treatment with selective CDK4/6 inhibitors are expected to be comparable to those seen with the use of exogenous growth factors (e.g., GCSF and erythropoietin). However, treatment with selective CDK4/6 inhibitor compounds should have many advantages in that it can ameliorate suppression of platelet and lymphocytes counts, which no previously reported treatment is capable of doing effectively. Thus, the presently disclosed methods can be used to mitigate chemo-induced thrombocytopenia and lymphopenia.

Further, treatment with selective CDK4/6 inhibitors will not force stem cells to proliferate at a faster rate. This can be desirable because enforced proliferation can increase late and long-term bone marrow toxicities seen in humans and mice after growth factor support intended to ameliorate the effects of DNA damage. See Herodin et al., Blood, 2003, 101, 2609-2616; Hershman et al., J. Natl. Cancer Inst., 2007, 99, 196-205; and Le Deley et al., J. Clin. Oncol., 2007, 25, 292-300. Several groups have reported that the use of G-CSF can significantly increase the incidence of late (>3 years post-chemo) bone marrow toxicity (for example, myelodysplasia) in cancer patients who survive the disease. Several groups have also reported that EPO and related erythrocytosis stimulating compounds appear to increase cancer-related mortality when given with chemotherapy. See Khuri, N. Engl. J. Med., 2007, 356, 2445-2448. While it is uncertain if this represents an ability of EPO to stimulate tumor growth or tumor angiogenesis, these findings point to a major liability of the use of EPO in the oncology setting. PQ is not expected to stimulate tumor growth and could be used safely in treatments to increase the red blood cell count in cases where the use of EPO is contraindicated.

Several other advantages can result from chemoprotective methods involving selective CDK4/6 inhibitors. The reduction in chemotoxicity afforded by the selective CDK4/6 inhibitors with regard to healthy cells is not expected to affect the efficacy of the chemotherapeutic compound in reducing the growth and proliferation in cancer cells. Further, the reduction in chemotoxicity is anticipated to allow for dose intensification (e.g., higher doses and/or more doses over a given period of time or a shorter period of time), which will translate to better efficacy. Therefore, the presently disclosed methods can result in chemotherapeutic regimens that are less toxic and more effective.

Also in contrast to protective treatments with exogenous biological growth factors, selective CDK4/6 inhibitors include many less expensive, orally available small molecules, which can be formulated for administration via a number of different routes. When appropriate, such small molecules can be formulated for oral, topical, intranasal, inhalation, intravenous or any other form of administration. In addition, as opposed to biologics, stable small molecules can be more easily stockpiled and stored. Thus, the selective CDK4/6 inhibitor compounds can be more easily and cheaply kept on hand in emergency rooms where subjects of accidental chemical exposure to cytotoxic (e.g., DNA damaging) compounds might report or at sites where chemical exposure is particularly likely to occur, including, chemical or drug manufacturing facilities and chemical research laboratories.

Selective CDK4/6 inhibitors can also be used in protecting healthy HSPCs during chemical treatments of abnormal tissues in non-cancer proliferative diseases, including but not limited to the following: hemangiomatosis in infants, secondary progressive multiple sclerosis, chronic progressive myelodegenerative disease, neurofibromatosis, ganglioneuromatosis, keloid formation, Paget's Disease of the bone, fibrocystic disease of the breast, Peronies and Duputren's fibrosis, restenosis and cirrhosis. Further, selective CDK4/6 inhbitors can be used to ameliorate the effects of DNA damaging (e.g., intercalating or alkylating) chemicals in the event of accidental chemical exposure or overdose (e.g., methotrexate overdose). Thus, the presently disclosed methods can be used to protect chemical plant workers, chemical researchers and emergency responders from occupational exposure, for example, in the event of a chemical spill.

According to the presently disclosed subject matter, chemotherapy can be administered to a subject on any schedule and in any dose consistent with the prescribed course of treatment, as long as the chemoprotectant compound is administered prior to, during, or following the administration of the chemotherapeutic. Generally, the chemoprotectant compound can be administered to the subject during the time period ranging from 24 hours prior to exposure with the chemotherapeutic compound until 24 hours following exposure. However, this time period can be extended to time earlier that 24 hour prior to exposure to the chemotherapeutic (e.g., based upon the time it takes the compound to achieve suitable plasma concentrations and/or the compounds plasma half-life). Further, the time period can be extended longer than 24 hours following exposure to the chemotherapeutic compound or other DNA damaging compound so long as later administration of the CDK4/6 inhibitor leads to at least some protective effect. Such post-exposure treatment can be especially useful in cases of accidental exposure or overdose.

In some embodiments, the selective CDK4/6 inhibitor can be administered to the subject at a time period prior to the administration of the chemotherapeutic agents, so that plasma levels of the selective CDK4/6 inhibitor are peaking at the time of administration of the chemotherapeutic compound. If convenient, the selective CDK4/6 inhibitor can be administered at the same time as the chemotherapeutic agent, in order to simplify the treatment regimen. In some embodiments, the chemoprotectant and chemotherapeutic compounds can be provided in a single formulation.

If desired, multiple doses of the chemoprotectant compound can be administered to the subject. Alternatively, the subject can be given a single dose of the selective CDK4/6 inhibitor. The course of chemotherapy and chemoprotectant treatment can differ from subject to subject, and those of ordinary skill in the art can readily determine the appropriate dose and schedule of chemotherapy and associated chemoprotectant treatment in a given clinical situation.

### III. Active Compounds, Salts and Formulations

As used herein, the term “active compound” refers to a selective CDK 4/6 inhibitor compound, or a pharmaceutically acceptable salt thereof. The active compound can be administered to the subject through any suitable approach. The amount and timing of active compound administered can, of course, be dependent on the subject being treated, on the dosage of DNA damaging compound to which the subject has been, is being, or is anticipated of being exposed to, on the manner of administration, on the pharmacokinetic properties of the active compound, and on the judgment of the prescribing physician. Thus, because of subject to subject variability, the dosages given below are a guideline and the physician can titrate doses of the compound to achieve the treatment that the physician considers appropriate for the subject. In considering the degree of treatment desired, the physician can balance a variety of factors such as age and weight of the subject, presence of preexisting disease, as well as presence of other diseases. Pharmaceutical formulations can be prepared for any desired route of administration, including but not limited to oral, intravenous, or aerosol administration, as discussed in greater detail below.

The therapeutically effective dosage of any specific active compound, the use of which is within the scope of embodiments described herein, can vary somewhat from compound to compound, and subject to subject, and can depend upon the condition of the subject and the route of delivery. As a general proposition, a dosage from about 0.1 to about 200 mg/kg can have therapeutic efficacy, with all weights being calculated based upon the weight of the active compound, including the cases where a salt is employed. In some embodiments, the dosage can be the amount of compound needed to provide a serum concentration of the active compound of up to between about 1-5 μM or higher. Toxicity concerns at the higher level can restrict intravenous dosages to a lower level, such as up to about 10 mg/kg, with all weights being calculated based on the weight of the active base, including the cases where a salt is employed. A dosage from about 10 mg/kg to about 50 mg/kg can be employed for oral administration. Typically, a dosage from about 0.5 mg/kg to 5 mg/kg can be employed for intramuscular injection. In some embodiments, dosages can be from about 1 μmol/kg to about 50 μmol/kg, or, optionally, between about 22 μmol/kg and about 33 μmol/kg of the compound for intravenous or oral administration.

In accordance with the presently disclosed methods, pharmaceutically active compounds as described herein can be administered orally as a solid or as a liquid, or can be administered intramuscularly, intravenously or by inhalation as a solution, suspension, or emulsion. In some embodiments, the compounds or salts also can be administered by inhalation, intravenously, or intramuscularly as a liposomal suspension. When administered through inhalation the active compound or salt can be in the form of a plurality of solid particles or droplets having a particle size from about 0.5 to about 5 microns, and optionally from about 1 to about 2 microns.

The pharmaceutical formulations can comprise an active compound described herein or a pharmaceutically acceptable salt thereof, in any pharmaceutically acceptable carrier. If a solution is desired, water is the carrier of choice with respect to water-soluble compounds or salts. With respect to the water-soluble compounds or salts, an organic vehicle, such as glycerol, propylene glycol, polyethylene glycol, or mixtures thereof, can be suitable. In the latter instance, the organic vehicle can contain a substantial amount of water. The solution in either instance can then be sterilized in a suitable manner known to those in the art, and typically by filtration through a 0.22-micron filter. Subsequent to sterilization, the solution can be dispensed into appropriate receptacles, such as depyrogenated glass vials. The dispensing is optionally done by an aseptic method. Sterilized closures can then be placed on the vials and, if desired, the vial contents can be lyophilized.

In addition to the active compounds or their salts, the pharmaceutical formulations can contain other additives, such as pH-adjusting additives. In particular, useful pH-adjusting agents include acids, such as hydrochloric acid, bases or buffers, such as sodium lactate, sodium acetate, sodium phosphate, sodium citrate, sodium borate, or sodium gluconate. Further, the formulations can contain antimicrobial preservatives. Useful antimicrobial preservatives include methylparaben, propylparaben, and benzyl alcohol. An antimicrobial preservative is typically employed when the formulation is placed in a vial designed for multi-dose use. The pharmaceutical formulations described herein can be lyophilized using techniques well known in the art.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch (e.g., potato or tapioca starch) and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules. Materials in this connection also include lactose or milk sugar as well as high molecular weight polyethylene glycols. When aqueous suspensions and/or elixirs are desired for oral administration, the compounds of the presently disclosed subject matter can be combined with various sweetening agents, flavoring agents, coloring agents, emulsifying agents and/or suspending agents, as well as such diluents as water, ethanol, propylene glycol, glycerin and various like combinations thereof.

In yet another embodiment of the subject matter described herein, there is provided an injectable, stable, sterile formulation comprising an active compound as described herein, or a salt thereof, in a unit dosage form in a sealed container. The compound or salt is provided in the form of a lyophilizate, which is capable of being reconstituted with a suitable pharmaceutically acceptable carrier to form a liquid formulation suitable for injection thereof into a subject. When the compound or salt is substantially water-insoluble, a sufficient amount of emulsifying agent, which is physiologically acceptable, can be employed in sufficient quantity to emulsify the compound or salt in an aqueous carrier. Particularly useful emulsifying agents include phosphatidyl cholines and lecithin.

Additional embodiments provided herein include liposomal formulations of the active compounds disclosed herein. The technology for forming liposomal suspensions is well known in the art. When the compound is an aqueous-soluble salt, using conventional liposome technology, the same can be incorporated into lipid vesicles. In such an instance, due to the water solubility of the active compound, the active compound can be substantially entrained within the hydrophilic center or core of the liposomes. The lipid layer employed can be of any conventional composition and can either contain cholesterol or can be cholesterol-free. When the active compound of interest is water-insoluble, again employing conventional liposome formation technology, the salt can be substantially entrained within the hydrophobic lipid bilayer that forms the structure of the liposome. In either instance, the liposomes that are produced can be reduced in size, as through the use of standard sonication and homogenization techniques. The liposomal formulations comprising the active compounds disclosed herein can be lyophilized to produce a lyophilizate, which can be reconstituted with a pharmaceutically acceptable carrier, such as water, to regenerate a liposomal suspension.

Pharmaceutical formulations also are provided which are suitable for administration as an aerosol by inhalation. These formulations comprise a solution or suspension of a desired compound described herein or a salt thereof, or a plurality of solid particles of the compound or salt. The desired formulation can be placed in a small chamber and nebulized. Nebulization can be accomplished by compressed air or by ultrasonic energy to form a plurality of liquid droplets or solid particles comprising the compounds or salts. The liquid droplets or solid particles should have a particle size in the range of about 0.5 to about 10 microns, and optionally from about 0.5 to about 5 microns. The solid particles can be obtained by processing the solid compound or a salt thereof, in any appropriate manner known in the art, such as by micronization. Optionally, the size of the solid particles or droplets can be from about 1 to about 2 microns. In this respect, commercial nebulizers are available to achieve this purpose. The compounds can be administered via an aerosol suspension of respirable particles in a manner set forth in U.S. Pat. No. 5,628,984, the disclosure of which is incorporated herein by reference in its entirety.

When the pharmaceutical formulation suitable for administration as an aerosol is in the form of a liquid, the formulation can comprise a water-soluble active compound in a carrier that comprises water. A surfactant can be present, which lowers the surface tension of the formulation sufficiently to result in the formation of droplets within the desired size range when subjected to nebulization.

As indicated, both water-soluble and water-insoluble active compounds are provided. As used herein, the term “water-soluble” is meant to define any composition that is soluble in water in an amount of about 50 mg/mL, or greater. Also, as used herein, the term “water-insoluble” is meant to define any composition that has a solubility in water of less than about 20 mg/mL. In some embodiments, water-soluble compounds or salts can be desirable whereas in other embodiments water-insoluble compounds or salts likewise can be desirable.

The term “pharmaceutically acceptable salts” as used herein refers to those salts which are, within the scope of sound medical judgment, suitable for use in contact with subjects (e.g., human subjects) without undue toxicity, irritation, allergic response, and the like, commensurate with a reasonable benefit/risk ratio, and effective for their intended use, as well as the zwitterionic forms, where possible, of the compounds of the presently disclosed subject matter.

Thus, the term “salts” refers to the relatively non-toxic, inorganic and organic acid addition salts of compounds of the presently disclosed subject matter. These salts can be prepared in situ during the final isolation and purification of the compounds or by separately reacting the purified compound in its free base form with a suitable organic or inorganic acid and isolating the salt thus formed. In so far as the compounds of the presently disclosed subject matter are basic compounds, they are all capable of forming a wide variety of different salts with various inorganic and organic acids. Although such salts must be pharmaceutically acceptable for administration to animals, it is often desirable in practice to initially isolate the base compound from the reaction mixture as a pharmaceutically unacceptable salt and then simply convert to the free base compound by treatment with an alkaline reagent and thereafter convert the free base to a pharmaceutically acceptable acid addition salt. The acid addition salts of the basic compounds are prepared by contacting the free base form with a sufficient amount of the desired acid to produce the salt in the conventional manner. The free base form can be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the presently disclosed subject matter.

Pharmaceutically acceptable base addition salts are formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine.

The base addition salts of acidic compounds are prepared by contacting the free acid form with a sufficient amount of the desired base to produce the salt in the conventional manner. The free acid form can be regenerated by contacting the salt form with an acid and isolating the free acid in a conventional manner. The free acid forms differ from their respective salt forms somewhat in certain physical properties such as solubility in polar solvents, but otherwise the salts are equivalent to their respective free acid for purposes of the presently disclosed subject matter.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide such as hydrochloric, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

### IV. Methods of Screening Compounds for Chemoprotective Activity

In some embodiments, the presently disclosed subject matter provides a method of selecting chemoprotective compounds. In particular, the presently disclosed subject matter provides methods of selecting chemoprotective compounds that can produce transient PQ in healthy cells, allowing for treatment of a tumor with a cytotoxic (e.g., DNA damaging) compound or other agent (e.g., ionizing radiation), but that do not produce long term (or other undesired) toxicity and which provide chemoprotection without prolonged pretreatment periods. In some embodiments, it is desirable to screen a test compound or compounds by conducting one or more cell-based assay(s). The use of a cell-based assay can confirm the efficacy of compounds having CDK4/6 inhibitory ability in non-cell-based assays and aid in excluding compounds that have undesirable off target effects. The cell-based assay screening methods described herein have been shown to be predictive of a compound's chemoprotective abilities in vivo.

In some embodiments, the presently disclosed subject matter provides a method for screening a compound for use in preventing the effects of a cytotoxic agent in healthy cells, the method comprising: contacting a cyclin-dependent kinase 4 (CDK4) and/or cyclin-dependent kinase 6 (CDK6)-dependent cell population with a test compound for a period of time; performing cell cycle analysis of the cell population; and selecting a test compound that selectively induces G1 arrest in the cell population.

In some embodiments, the selecting comprises selecting a test compound that induces substantially pure G1 arrest (i.e., substantially no G2/M or S phase arrest or less than 20, 15, 12, 10, 8, 6, 5, 4, 3, 2, or 1% G2/M and/or S phase arrest).

Suitable test compounds include a variety of different compounds. For example, the test compounds can be compounds known or suspected of having CDK4/6 inhibitory effects. Test compounds can include those with known CDK4/6 inhibitory effects as measured via cell-free assays. In some embodiments, the test compounds can be selected from the group including, but not limited to, pyrido[2,3-d]pyrimidines (e.g., pyrido[2,3-d]pyrimidin-7-ones and 2-amino-6-cyano-pyrido[2,3-d]pyrimidin-4-ones), triaminopyrimidines, aryl[a]pyrrolo[3,4-d]carbazoles, nitrogen-containing heteroaryl-substituted ureas, 5-pyrimidinyl-2-aminothiazoles, benzothiadiazines, acridinethiones, and isoquinolones, such as the compounds described hereinabove.

Cell populations suitable for use according to the presently disclosed methods include, but are not limited to, telomerized human diploid fibroblasts (tHDFs) or CDK4/6-dependent cancer cell lines. In some embodiments, the CDK4/6-dependent cancer cell line is a cancer cell line lacking INK4a/ARF. In some embodiments, the cell population can be contacted with the test compound for 24 hours or less (e.g., 24, 23, 22, 21, 20, 19, 18, 17, 16, 15, 14, 13, 12, 11, 10, 9, 8, 7, 6, 5, 4, 3, 2, or 1 hours) prior to performing cell cycle analysis of the cell population. Any suitable amount of test compound can be used to contact the cell population. For example, the amount of test compound used to contact the cell population can be based upon known data related to the compound (e.g., known IC₅₀'s determined via cell-free kinase inhibition studies). Screening can further involve treating a plurality of cell populations with a test compound, wherein each of the plurality of cell populations is treated with a different amount of a particular test compound, in order to determine dosage-dependent effects.

After the cell population has been in contact with the test compound for the desired period of time, cell cycle analysis is performed to determine the percentage (%) of cells in a particular cell phase (e.g., G1, G2/M, S) or phases. For comparison, cell cycle analysis can also be performed in a cell population that was not treated with the test compound.

Methods of assessing the cell phase of a population of cells are known in the art and described, for example, in U.S. Patent Application Publication No. 2002/0224522. Cell phase can be assessed in a variety of ways including cytometric analysis, microscopic analysis, gradient centrifugation, elutriation and fluorescence techniques including immunofluorescence (which can be used in combination with, for example, any of the preceding techniques). Cytometric techniques include exposing the cell to a labelling agent or stain, such as DNA-binding dyes, e.g., propidium iodide (PI), and analyzing cellular DNA content by flow cytometry. Immunofluorescence techniques include detection of specific cell cycle indicators such as, for example, thymidine analogs (e.g., 5-bromo-2-deoxyuridine (BrdU) or an iododeoxyuridine), with fluorescent antibodies.

In one method of cell phase analysis using flow cytometry, the nuclear DNA content of a cell can be quantitatively measured at high speed as an indicator of cell cycle phase. DNA content is a marker of cell phase because the DNA content of a cell changes between the several phases of the cell cycle. Cells in G0/1 phase have DNA content set equal to 1 unit of DNA; cells in S phase duplicate DNA, increasing its content in proportion to progression through S; and upon entering G2 and then M phases, cells have twice the G0/1 phase DNA content (i.e., 2 units of DNA). Thus, S phase cells have a DNA content that is intermediate between that of cells in G1 and G2/M (which have twice as much DNA as cells in G1). Univariate analysis of cellular DNA content allows discrimination of G0/1, S and G2/M phase cells.

Flow cytometry measurement of cellular DNA content typically involves addition of a dye that binds stoichiometrically to DNA in a suspension of permeabilized cells or nuclei. Generally, cells are fixed or permeabilized, e.g., with a detergent, and then stained with a DNA-binding dye. Examples of such dyes include, but are not limited to, a nucleic acid-specific fluorochrome, propidium iodide (PI) or 4′6′-diamidino-2-phenylindole (DAPI). PI stains RNA in addition to DNA; thus, to avoid inclusion of measurement of fluorescence due to RNA in determining DNA content of a cell, it can be desirable to remove RNA by incubation with RNase. The DNA-bound PI emits red fluorescence when excited with blue light (488 nm). The DAPI-DNA complex can be excited by ultraviolet (UV) light (360 nm) and emits blue fluorescence. DNA can also be stained in live cells with the UV light-excitable fluorochrome Hoeschst 33242 which also emits blue fluorescence. Other DNA-binding dyes include, but are not limited to, Hoechst 33258, 7-AAD, LDS 751, and SYTO 16 (see, e.g., Molecular Probes Handbook of Fluorescent Probes and Research Chemicals, Haugland, Sixth Ed.; chapters 8 and 16 in particular). Generally, DNA-binding dyes are taken up passively by the cell and bind to DNA by intercalation, although some DNA-binding dyes are major or minor groove binding compounds.

The stained material incorporates an amount of dye proportional to the amount of DNA. The stained material is then measured in the flow cytometer and the emitted fluorescent signal yields an electronic pulse with a height (amplitude) proportional to the total fluorescence emission from the cell. The results of fluorescence measurements can also be displayed as cellular DNA content frequency histograms which show the proportions of cells in the various phases of the cycle based on differences in fluorescence intensity. Software containing mathematical models that fit the DNA histogram of a singlet have been developed to calculate the percentages of cells occupying the different phases of the cell cycle. Several manufacturers provide software for cell cycle analysis, including, for example, CELLFIT™ (Becton, Dickinson and Company, Franklin Lakes, N.J., United States of America).

Various nucleic acid analogs can be incorporated into DNA during cell replication. For example, BrdU is incorporated into DNA during replication in cells exposed to the analog. DNA that has incorporated the analog can be detected immunocytochemically using fluorescein-tagged anti-BrdU antibodies. DNA content can be assessed, for example, by counterstaining with a red fluorescing intercalating fluorochrome such as, for example, PI or 7-aminoactinomycin D (7-AAD). Bivariate analysis of DNA content versus immunofluorescence of anti-BrdU antibody distinguishes S phase cells on the basis of their difference in DNA content from G1 or G2/M cells and also based on incorporation of the green fluorescing anti-BrdU antibodies.

Centrifugation and centrifugal elutriation can be used to fractionate cells according to their size. Because cells in different phases differ in size, these methods can also be used to sort cells by cell phase and to thereby assess the phase of a cell. For example, early G1 phase cells are about half the size of mitotic or late G2 cells.

Chromosomes undergo morphological, ultrastructural and topological changes during progression of the cell cycle. Thus, chromosomes from cells in different phases of the cell cycle can be distinctive. The topology of a chromosome differs at different phases of the cell cycle. Interphase chromosomal DNA exists in various decondensed states to facilitate gene expression. Chromatin in chromosomal regions that is not being transcribed exist predominantly in the condensed form while regions being transcribed assume an extended form. Within the S phase, chromosomal DNA is further dispersed as it unwinds during the replication process. Upon conclusion of the S phase cohesion occurs to keep extended sister chromatids tightly associated. Typically, chromosomes begin to condense during prophase, undergoing several orders of supercoiling guided by histones and other facilitator proteins. Chromosomes are most dense during metaphase and begin to decondense again during telophase as the sister cells divide and normal transcription levels resume. Accordingly, cell phase can be assessed via various imaging (e.g., microscopy) techniques.

According to the presently disclosed methods, cell cycle analysis can be performed using any suitable technique, such as, but not limited to, flow cytometry, fluorimetry, cell imaging, and fluorescence spectroscopy or combinations thereof. In some embodiments, the cell cycle analysis comprises flow cytometry. In some embodiments, cell cycle analysis comprises labelling the cell population (e.g., following contact with the test compound for a period of time) with one or more labeling agent (e.g., DNA-binding agent or cell cycle indicator). In some embodiments, the labeling agent is BrdU, PI, or a combination thereof.

In some embodiments, the method of selecting a chemoprotective compound can further include one or more additional confirmatory assays. For example, in some embodiments, the method further comprises testing the ability of a test compound to induce G1 cell cycle arrest in CDK4/6-independent cells. Thus, in some embodiments, the method further comprises: contacting a second cell population with the test compound that selectively induces G1 arrest in CDK4/6-dependent cells for a period of time, wherein the second cell population comprises CDK4- and/or CDK6-independent cells; performing cell cycle analysis in the second cell population; and selecting a test compound that is free of selective induction of G1 arrest in the second cell population.

In some embodiments, the second cell population is a cancer cell line, such as a cancer cell line associated with a cancer that is present in a subject to be treated with the chemoprotective compound. In some embodiments, the second cell population is retinoblastoma tumor suppressor protein (RB)-null. In some embodiments, the second cell population is a cell population characterized by increased activity of CDK1 or CDK2, high levels of MYC expression, increased cyclin E or increased cyclin A.

The method can also include confirming that the selected test compound reduces DNA damage and/or maintains cell viability in a cell population contacted with a cytotoxic (e.g., DNA damaging) compound. For example, the prevention of DNA damage and/or maintenance of cell viability can be assessed in an ex vivo cell population (e.g., a cell population maintained in culture) prior to the chemoprotective agent being used in vivo.

Accordingly, in some embodiments, the confirmatory assay comprises, contacting a cell population with a test compound for a period of time or as a single dose at a point in time prior to, at the same time as, or following contact of the cell population with a cytotoxic compound (e.g., a chemotherapeutic compound). In some embodiment, the cytotoxic compound is a DNA damaging compound. In some embodiments, the DNA damaging compound used according to the presently disclosed method is doxorubicin, etoposide, carboplatin, or combinations thereof.

Reduction in DNA damage effected by the test compound or maintenance of cell viability effected by the test compound in cells treated with cytotoxic compounds can be assessed in any suitable manner. For example, DNA damage in cell populations can be assessed by performing a gamma-H2AX assay as described further herein below. Mammalian cells respond to agents that introduce DNA double-stranded breaks with the immediate and substantial phosphorylation of histone H2AX. Thus, detection of the phosphorylated H2AX, termed gamma-H2AX (γH2AX), using commercially available antibodies, can serve as a measure of DNA damage in cells.

A variety of cell proliferation assays are also known in the art to assess cell viability. In some embodiments, cell viability is assessed by performing a assay using 2-(4-iodophenyl)-3-(4-nitrophenyl)-5-(2,4-disulfophenyl)-2H-tetrazolium monosodium salt (WST-1), such as described further hereinbelow. In the WST-1 assay, WST-1 is cleaved by a mitochondrial reductase present in viable cells to form a colored product (i.e., formazan) that can be detected by measuring absorbance at a particular wavelength (e.g., 420-480 nm). Other tetrazolium salts that can be used as colorometric substrates include WST-8, TTC, INT, MTS, MTT and XTT. The CellTiter-Glo® assay (CTG assay; Promega, Madison, Wis., United States of America) measures cell viability by measuring ATP concentration in cell lysate. Cell viability can also be assessed by measuring DNA synthesis (e.g. by incorporation of nucleic acid analogs), and other techniques known in the art.

### EXAMPLES

The following Examples provide illustrative embodiments. In light of the present disclosure and the general level of skill in the art, those of skill can appreciate that the following Examples are intended to be exemplary only and that numerous changes, modifications, and alterations can be employed without departing from the scope of the presently disclosed subject matter.

### Methods

Compounds: The compounds used in the following studies are shown in Table 1, below. With the exception of flavopiridol, the compounds were freshly synthesized via known literature routes or purchased from commercial sources. Flavopiridol was provided by Dr. Kwok-Kin Wong (Dana-Farber Cancer Institute, Harvard Medical School, Boston, Mass., United States of America). Roscovitine and Genistein were purchased from LC Laboratories (Woburn, Mass., United States of America). 2BrIC was freshly synthesized for use in the present studies by OTAVA chemicals (Kiev, Ukraine), but is also commercially available from, for example, OTAVA Chemicals (Kiev, Ukraine) and Alexis Biochemicals (EnzoLife Sciences, Inc., Farmingdale, N.Y., United States of America). 2BrIC can be synthesized according to methods described in Zhu et al., J. Med. Chem., 46, 2027-2030 (2003). PD 0332991 was synthesized as described below in Example 1. The structure and purity of all compounds was confirmed by NMR and LC-MS. All compounds were >94% pure.

TABLE 1
Selective and Non-Selective CDK4/6 Inhibitor Compounds.

| Compound | Structure |
|---|---|
| 1 | 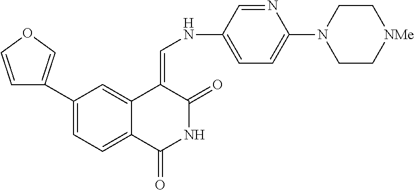 |
| 2 | 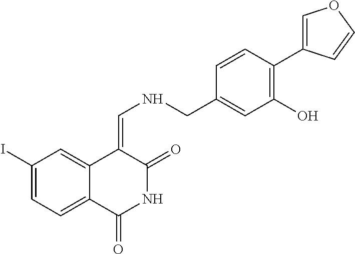 |
| 3 | 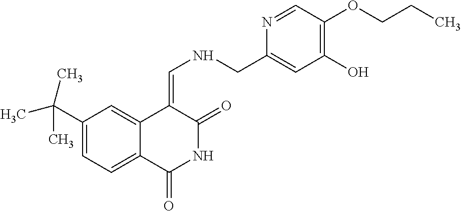 |
| 4 | 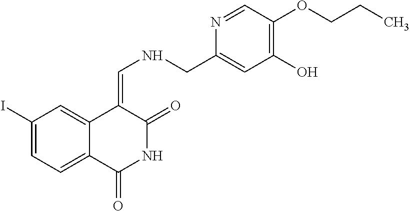 |
| 2BrIC | 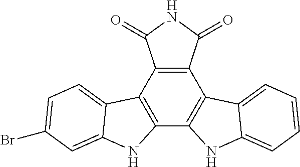 |
| 5 | 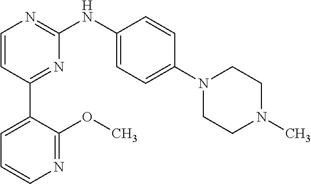 |
| 6 | 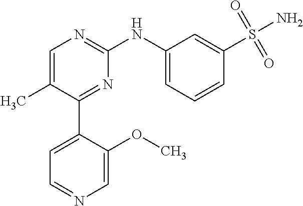 |
| 7 (R547) | 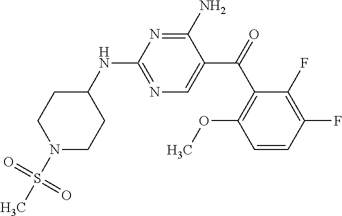 |
| PD (PD 0332991) | 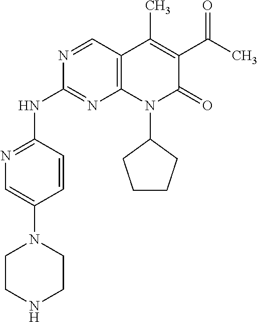 |
| flavopiridol | 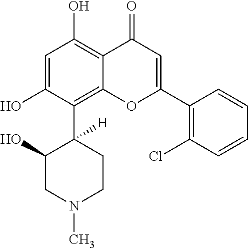 |
| Roscovitine | 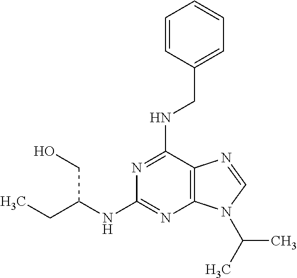 |
| Genistein | 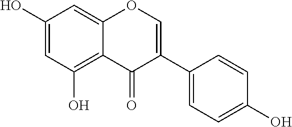 |
| 8 | 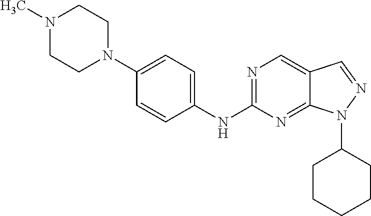 |
| 9 | 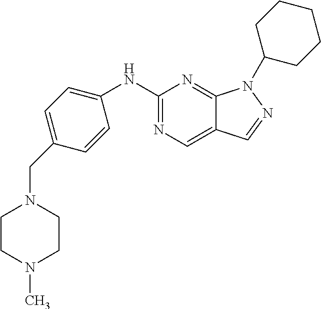 |
| 10 | 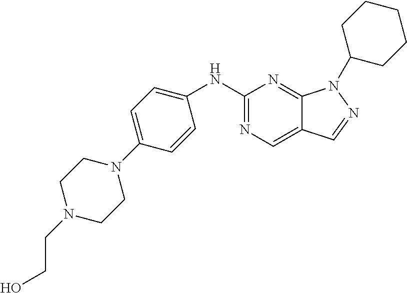 |
| 11 | 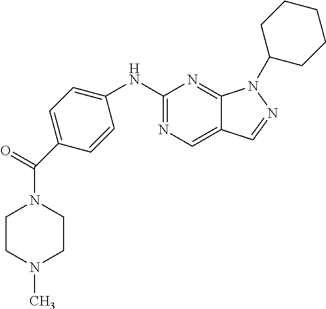 |
| 12 | 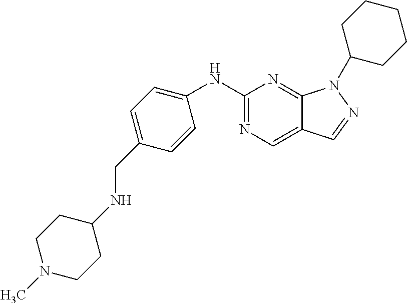 |
| 13 | 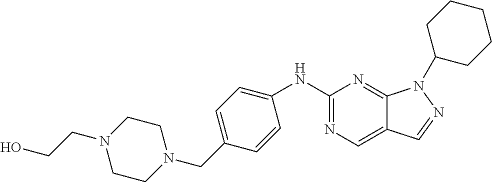 |
| 14 | 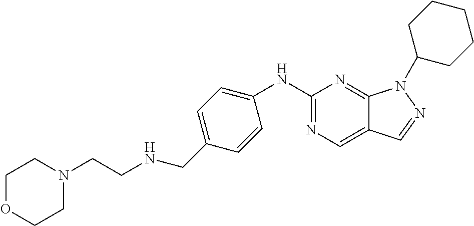 |
| 15 | 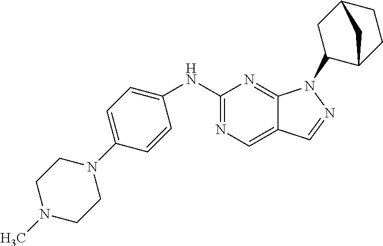 |

Cell lines: Telomerized human diploid fibroblast (tHDF) cells (HS68) were cultured in Dulbecco's Modified Eagle Medium (DMEM) +10% fetal bovine serum (FBS) with any additional compounds. The same conditions were used for A2058 and WM2664, human melanoma cell lines with known RB-pathway mutations: A2058 is RB-null, whereas WM2664 lacks 1NK4a/ARF. Thus, A2058 cells are CDK4/6-independent, while WM2664 cells are CDK4/6-dependent. Cells were cultured in DMEM+10% FBS.

Cell cycle analysis: Cell cycle analysis was performed using BrdU and propidium iodide (both from BD Biosciences Pharmigen, San Jose, Calif., United States of America) following the manufacturer's protocol. Cells were treated for 24 hours with a test compound at a desired dose prior to 15 minutes BrdU pulse, cell harvesting, fixation, staining, and analysis by flow cytometry. Histograms of dose-response curves of PD332991 and 2BrIC in HS68, WM2664 and A2058 cells were analyzed using Mod-Fit™ software from Verity Software House (Topsham, Me., United States of America).

γH2AX assay: For γH2AX assay, cells were treated with a dose response of PD332991 or 2BrIC for 24 hours. Cells were fixed, permeabilized, and stained with anti-γH2AX antibody as per γH2AX Flow Kit (Millipore, Billerica, Mass., United States of America). γH2AX levels were assessed by flow cytometry.

Cell proliferation assays: Cell proliferation assays were performed by seeding 1×10³ cells per well in a 96-well tissue culture plate in 100 μL of growth medium. Cells were treated as indicated with compound from Table 1 and doxorubicin, etoposide or carboplatin. Following treatment, cells were allowed to recover for 7 days in normal growth medium. At the end of the recovery period, cell number was quantified using the WST-1 cell proliferation assay (TaKaRa Bio USA, Madison, Wis., United States of America) or the CellTiter-Glo® assay (CTG; Promega, Madison, Wis., United States of America). Data is presented as absorbance at 450 nM for the WST assays or Relative Light Units (RLU) for the CTG assays.

In Vivo Pharmacodynamic Assay (BrdU Incorporation):

### Treatments:

PD0332991: For HSPC proliferation experiments, mice received daily oral gavage with PD0332991 150 mg/kg for 2 days with 1 mg BrdU intraperitoneal injection (i.p.) every 6 hours for 24 hours prior to sacrifice.

2BrIC: For HSPC proliferation experiments, mice were treated with two doses of 2-bromo-12,13-dihydro-5H-indolo[2,3-a]pyrrolo[3,4-c]carbazole-5,7(6H)-dione (2BrIC) 300 mg/kg oral gavage or vehicle control. 2BrIC was solubilized for oral gavage using formulation #6 from the Hot Rod formulation kit (Pharmatek, Inc., San Diego, Calif., United States of America). 2BrIC was administered 2 hours prior to BrdU administration and readministered at the time of the BrdU 1 mg i.p. injection. After BrdU+/−2BrIC treatment for the indicated times, the mice were sacrificed and bone marrow harvested for immunophenotyping, and BrdU analysis.

### Analysis of BrdU Incorporation by Flow Cytometry:

Bone marrow (BM) was harvested from femurs of mice, pooled, and centrifuged to purify bone marrow mononuclear cells (BM-MNCs). Cells were then incubated for 5 minutes in ACK buffer to lyse red blood cells. All antibodies were from BD Pharmingen (San Jose, Calif., United States of America) unless otherwise indicated. Purified BM-MNCs were incubated with mouse lineage mixture biotin conjugated antibody, followed by streptavidin-FITC. Cells were then stained with Sca-1-PE-Cy7 and c-kit-APC-alexa750 antibodies. Cell viability was assessed using LIVE/DEAD Aqua Dead Cell Stain Kit (Invitrogen Corporation, Carlsbad, Calif., United States of America). For BrdU incorporation assay, the cells were fixed, permeablized, and stained with an APC BrdU Flow Kit according to the manufacturer's instruction. In all experiments, PE-Cy7, FITC, APC-alexa750, and the Aqua Dead cell stain isotype controls were included as appropriate. Flow cytometric analysis was performed using a CyAn ADP (Dako, Glostrup, Denmark). For each sample, a minimum of 500,000 cells was analyzed and the data were analyzed using FlowJo software (Tree Star, Ashland, Oreg., United States of America).

Myelosuppression Assay: Weekly Complete Blood Counts:

### Treatments:

PD0332991: In the carboplatin experiments, mice were treated with a single dose of PD0332991 150 mg/kg oral gavage or vehicle control followed by carboplatin 100 mg/kg IP injection. In the doxorubicin experiments, mice were treated with a PD0332991 150 mg/kg by oral gavage or vehicle control one hour before doxorubicin 10 mg/kg by IP injection on day 0 and then repeated on day 7.

2BrIC: Mice were treated with a single dose of carboplatin 100 mg/kg by IP injection and two doses of 2BrIC 150mg/kg oral gavage or vehicle control. Mice were pretreated with 2BrIC two hours prior to carboplatin administration and then readministered a second dose of 2BrIC at the time of the carboplatin injection.

### Blood Collection and Platelet Quantification:

Baseline complete blood count (CBC) analysis were performed on a subset of mice prior to drug administration. Following drug administration (chemotherapy+/−indicated CDK4/6 inhibitor or control), mice were monitored weekly for the presence of myelosuppression by CBC analysis. CBC analysis was performed using BD Microtainer tubes with K2E (K₂EDTA), 40 μL of blood was collected by tail vein nick. Blood was analyzed using a HESKA CBC-Diff Veterinary Hematology System. CBC analysis included measurement of white blood cells, lymphocytes, granulocytes, monocytes, hematocrit, red blood cells, hemoglobin, platelets, and other common hematological parameters. TOXILIGHT™ assay:

Cellular cytotoxicity was assessed using the TOXILIGHT™ Bioassay kit (Lonza, Basel, Switzerland) which measures cytolysis by quantifying the release of adenylate kinase into the culture media. Briefly, 20 μL was aspirated from each well of 96 well plates of cells treated with varying concentrations of PD 0332991 or staurosporine. 100 μL of TOXILIGHT™ reagent is added and incubated for 5 minutes and read in a luminometer at 1 second/well.

### Example 1

### Synthesis of PD

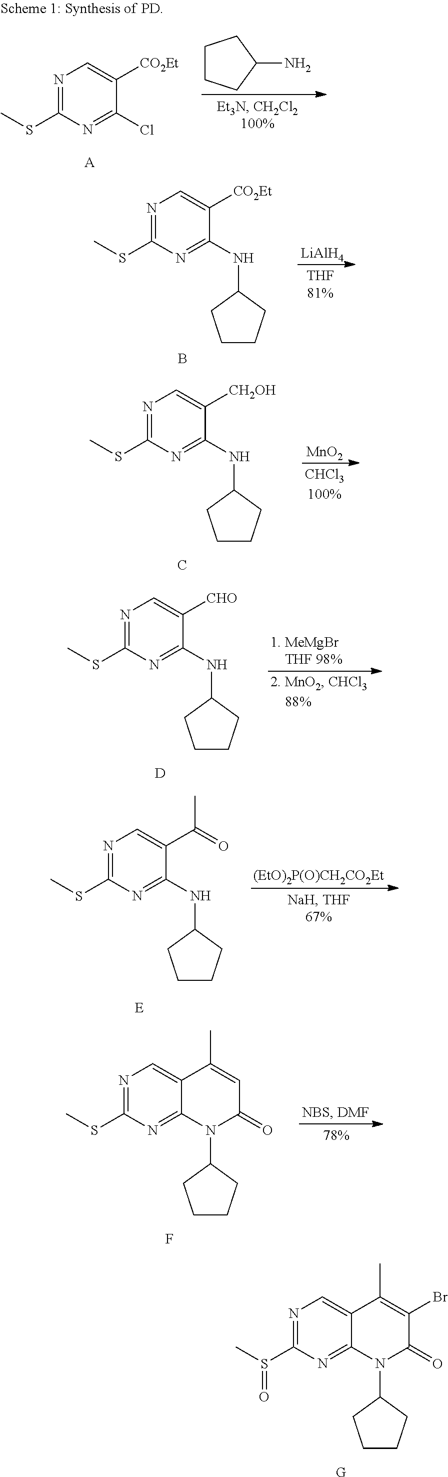

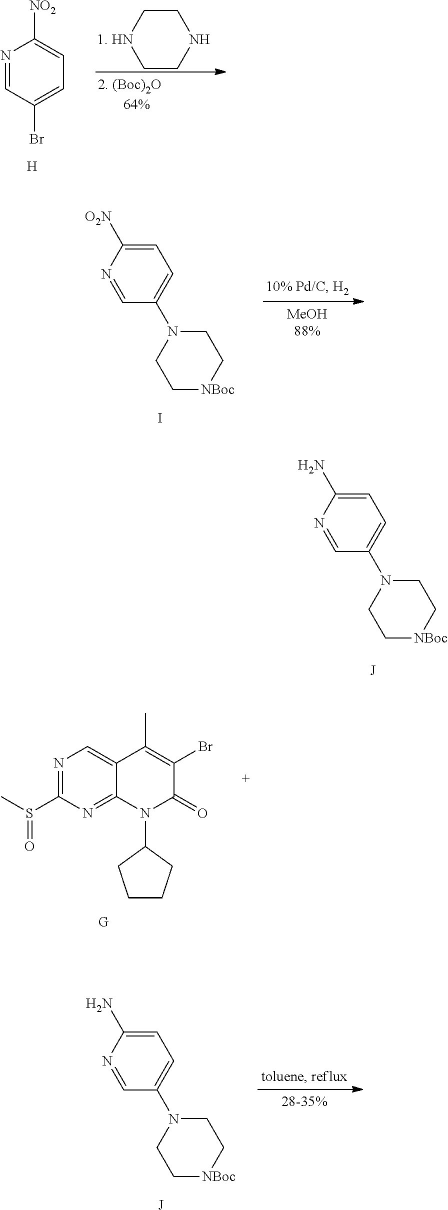

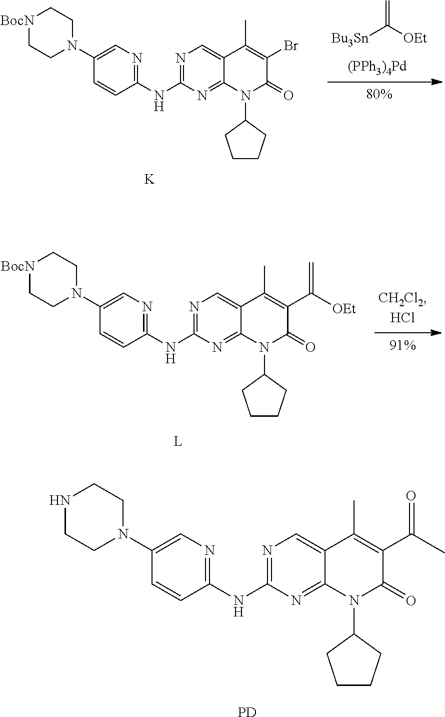

PD was synthesized as shown above in Scheme 1. Reactions shown in Scheme 1 generally followed previously reported procedures (see VandelWel et al., J. Med Chem., 48, 2371-2387 (2005); and Toogood et al., J. Med. Chem., 48, 2388-2406 (2005)) , with the exceptions of the reaction converting compound D to compound E and the reaction converting compound F to compound G.

Conversion of Compound D to Compound E:

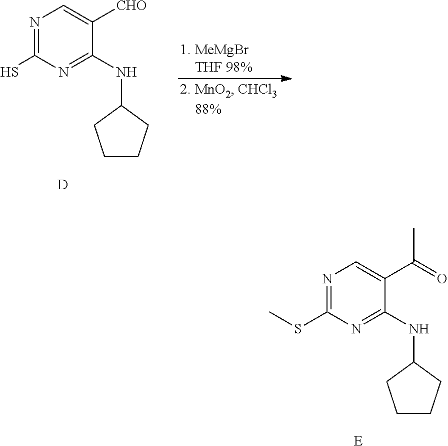

Compound D (40 g, 169 mmol) was dissolved in anhydrous THF (800 mL) under nitrogen and the solution was cooled in ice bath, to which MeMgBr was added slowly (160 mL, 480 mmol, 3 M in ether) and stirred for 1 h. The reaction was quenched with saturated aqueous NH₄Cl the partitioned between water and EtOAc. The organic layer was separated and the aqueous layer was extracted with EtOAc. The combined organic were washed with brine and dried over MgSO₄. Concentration gave an intermediate product as an oil (41.9 g, 98%).

The above intermediate (40 g, 158 mmol) was dissolved in dry CHCl₃ (700 mL). MnO₂(96 g, 1.11 mol) was added and the mixture was heated to reflux with stirring for 18 h and another MnO₂ (34 g, 395 mmol) was added and continue to reflux for 4 h. The solid was filtrated through a Celite pad and washed with CHCl₃. The filtrate was concentrated to give a yellow solid compound E (35 g, 88%), Mp: 75.8-76.6° C.

Conversion of Compound F to Compound G:

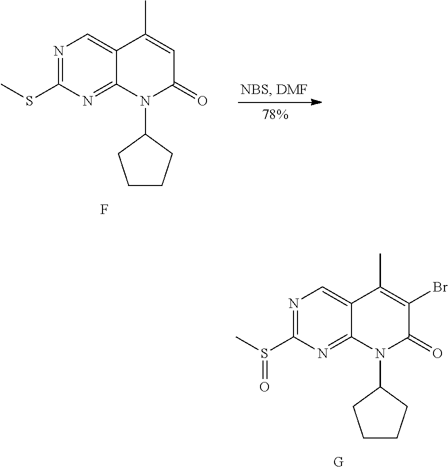

Compound F (5 g, 18.2 mmol) was dissolved in anhydrous DMF (150 mL) and NBS (11.3 g, 63.6 mmol) was added. The reaction mixture was stirred at r.t. for 3.5 h and then poured into H₂O (500 mL), the precipitate was filtered and washed with H₂O. The solid recrystallized from EtOH to give compound G as a white solid (5.42 g, 80.7%), mp: 210.6-211.3° C.

Characterization Data for PD:

LC-MS: 448.5 (ESI, M+H). Purity: ˜99%

¹H NMR(300 MHz, D₂O): 9.00(s, 1H), 8.12 (dd, J=9.3 Hz, 2.1 Hz, 1H), 7.81(d, J=2.4 Hz, 1H), 7.46(d, J=9.6 Hz, 1H), 5.80-5.74 (m, 1H), 3.57-3.48(m, 8H), 2.48(s, 3H), 2.37(s, 3H), 2.13-1.94(m, 6H), 1.73-1.71(m, 2H).

¹³C NMR (75 MHz, D₂O): 203.6, 159.0, 153.5, 153.3, 152.2, 139.9, 139.4, 139.2, 133.1, 129.0, 118.7, 113.8, 107.4, 51.8, 42.2, 40.0, 28.0, 25.2, 22.6, 10.8.

### Example 2

### Selective G1 Arrest in CDK4/6-Dependent Cell Lines

Several human cell lines were exposed to numerous small molecule kinase inhibitors. Cell cycle analysis was performed as described in the Methods section hereinabove.

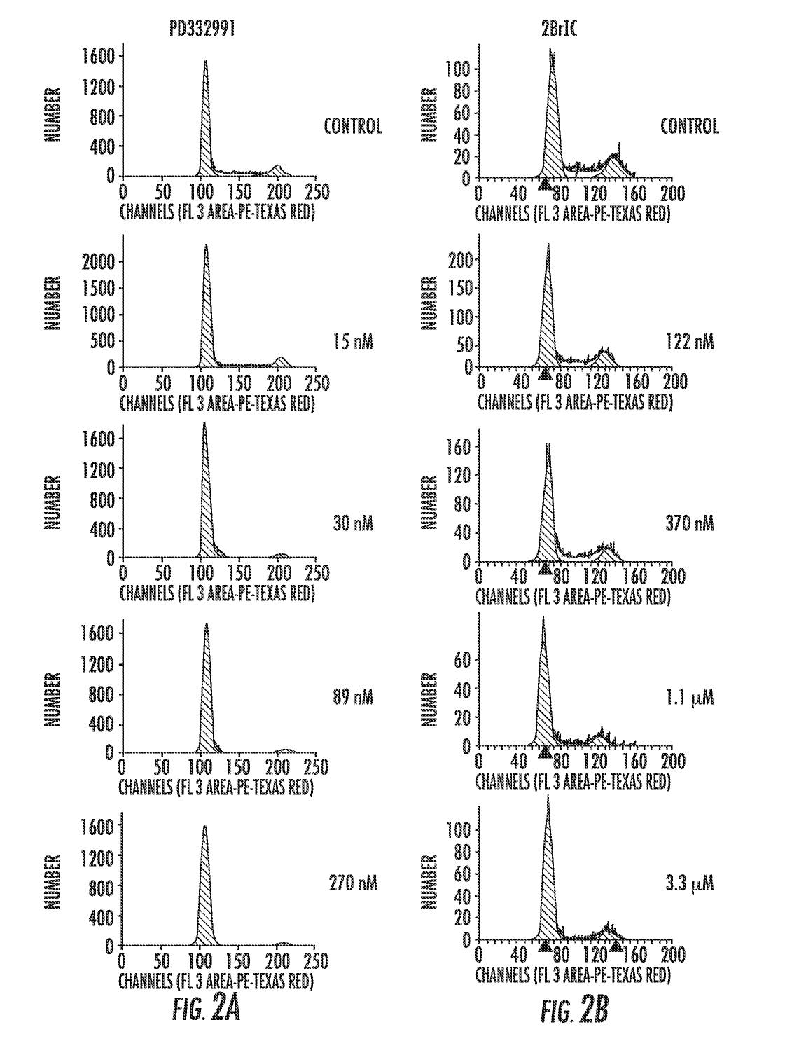

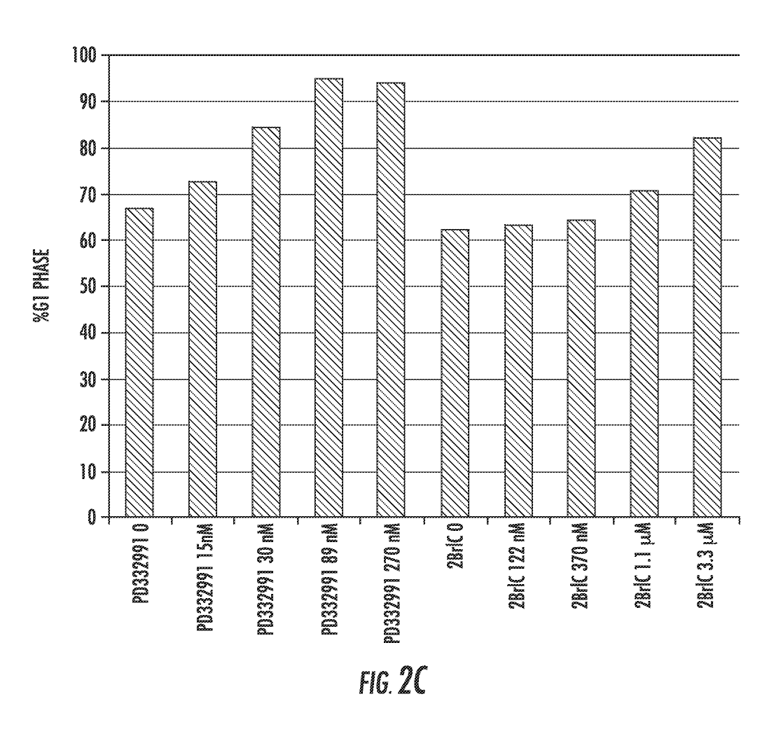

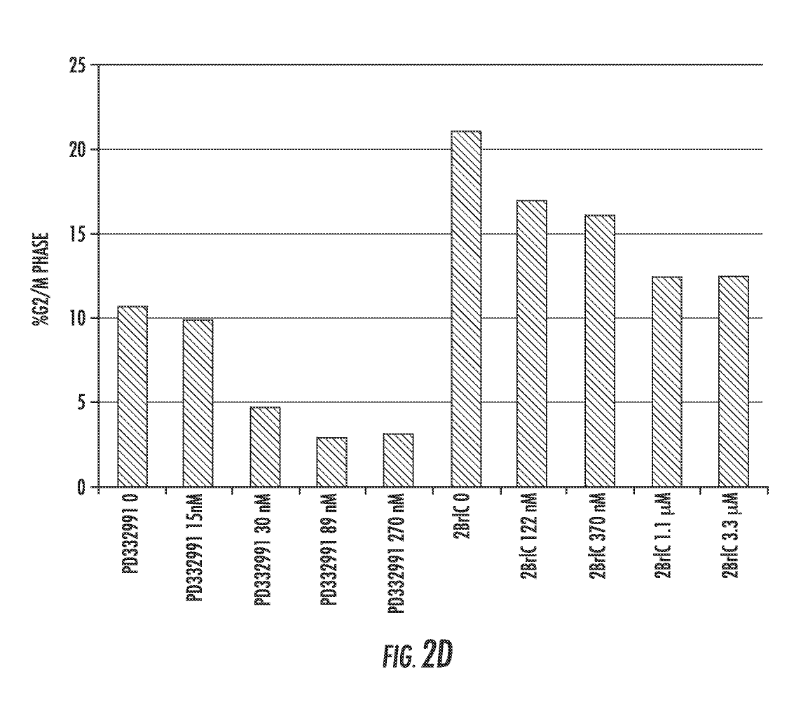

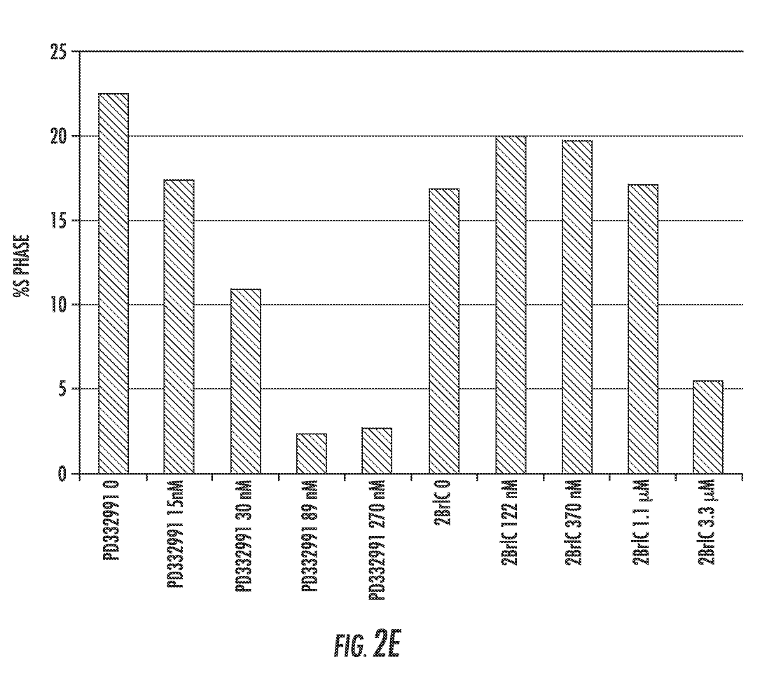

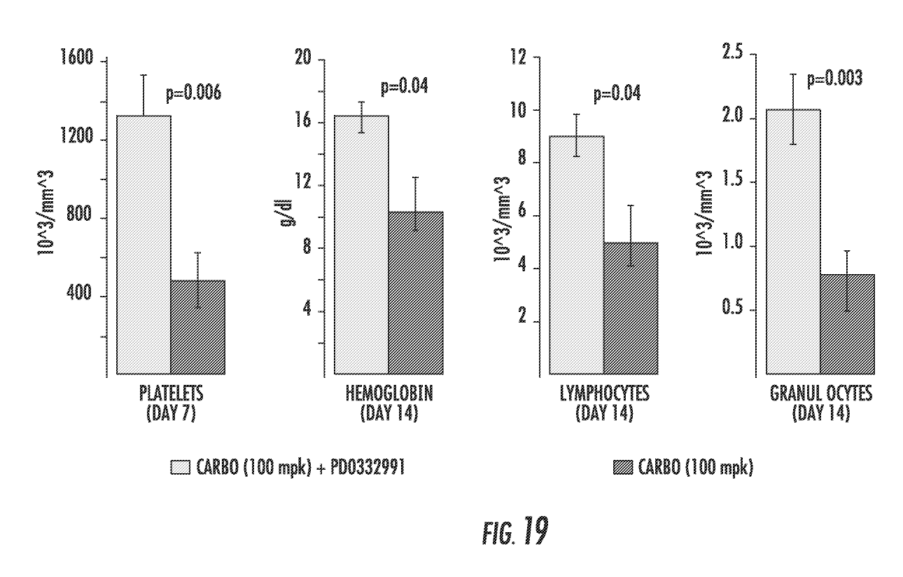

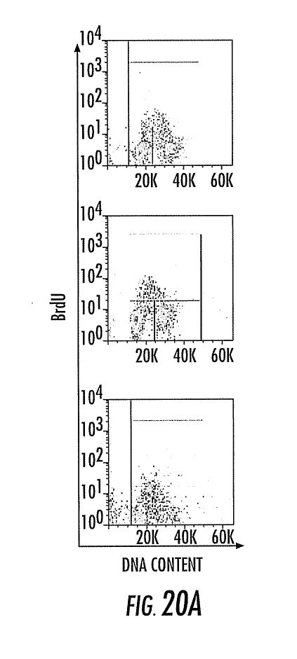

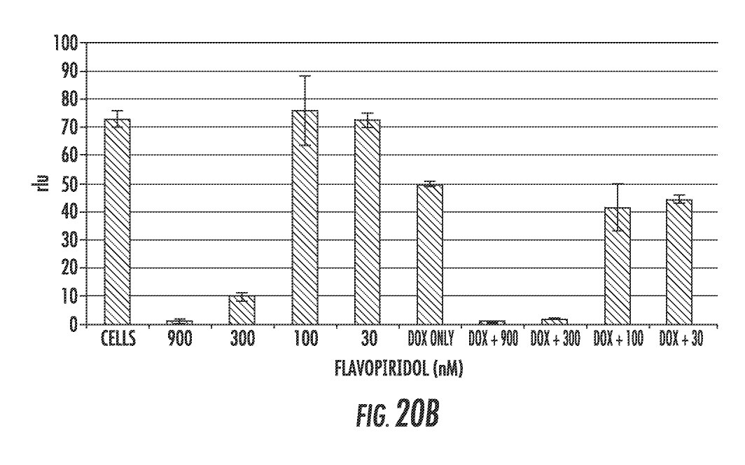

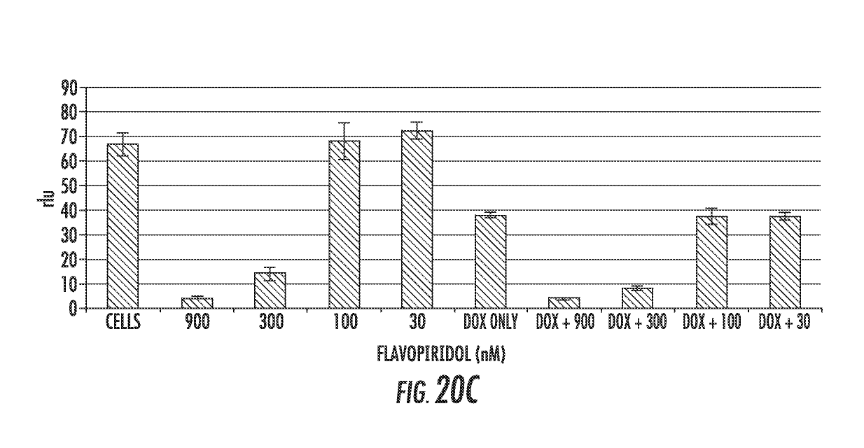

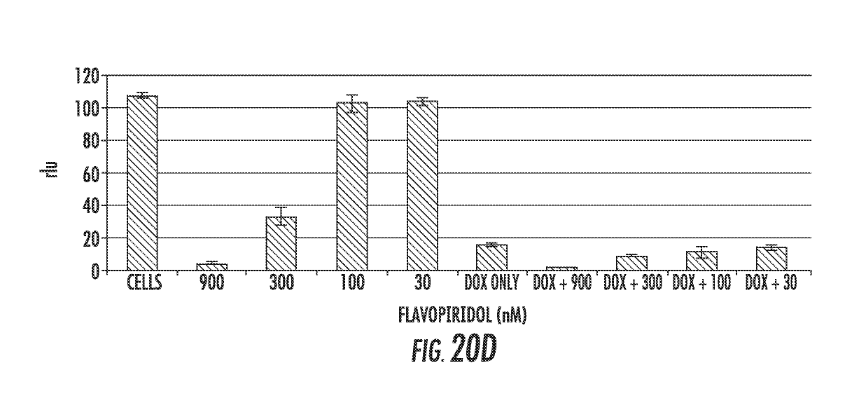

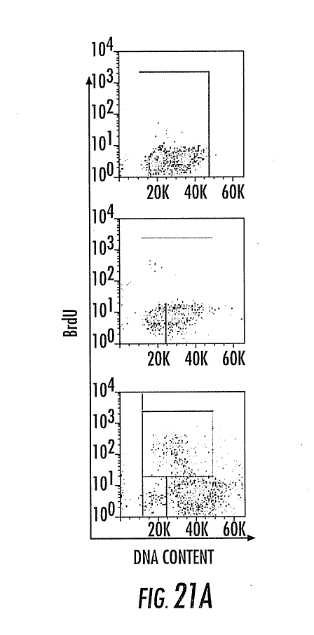

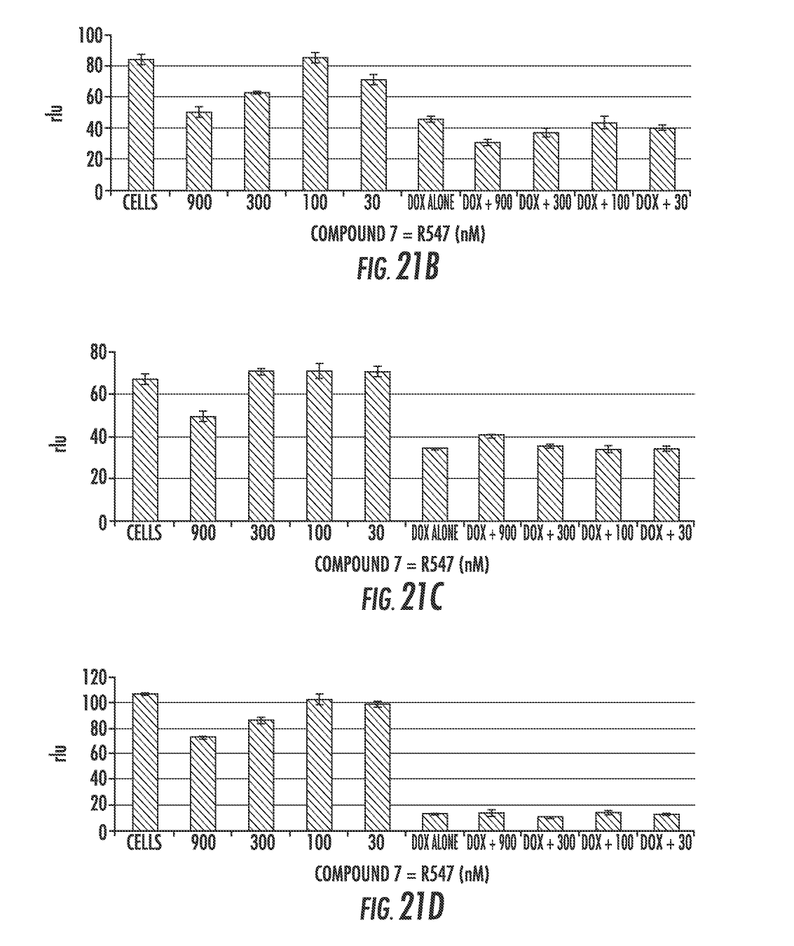

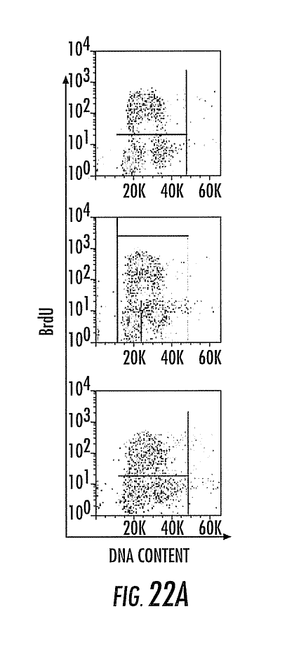

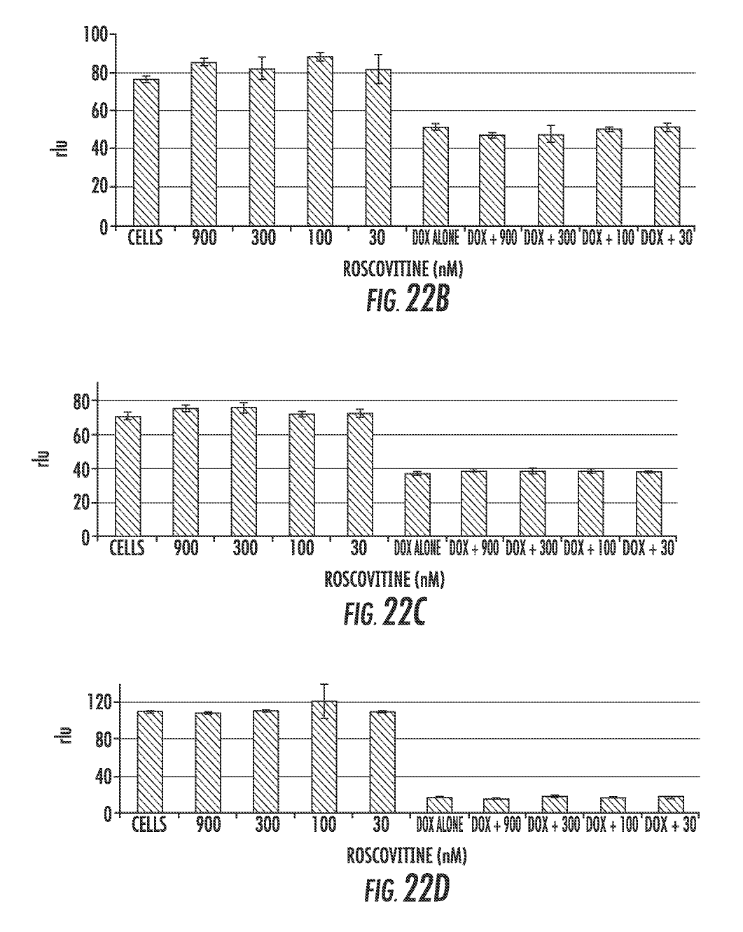

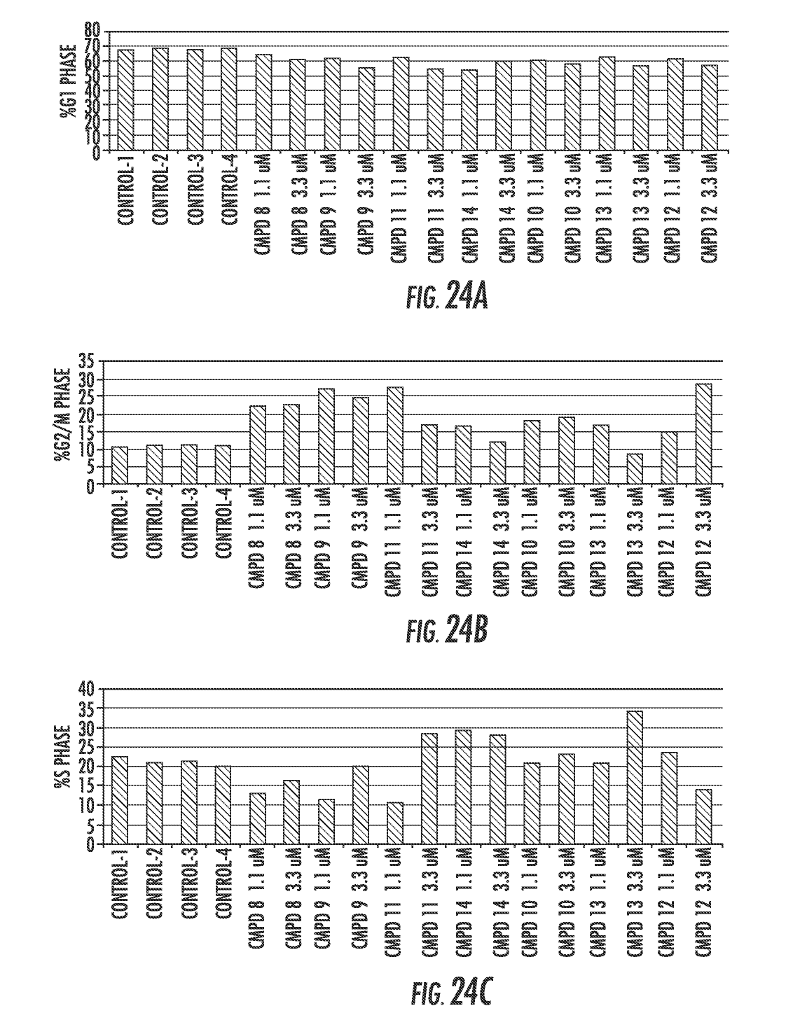

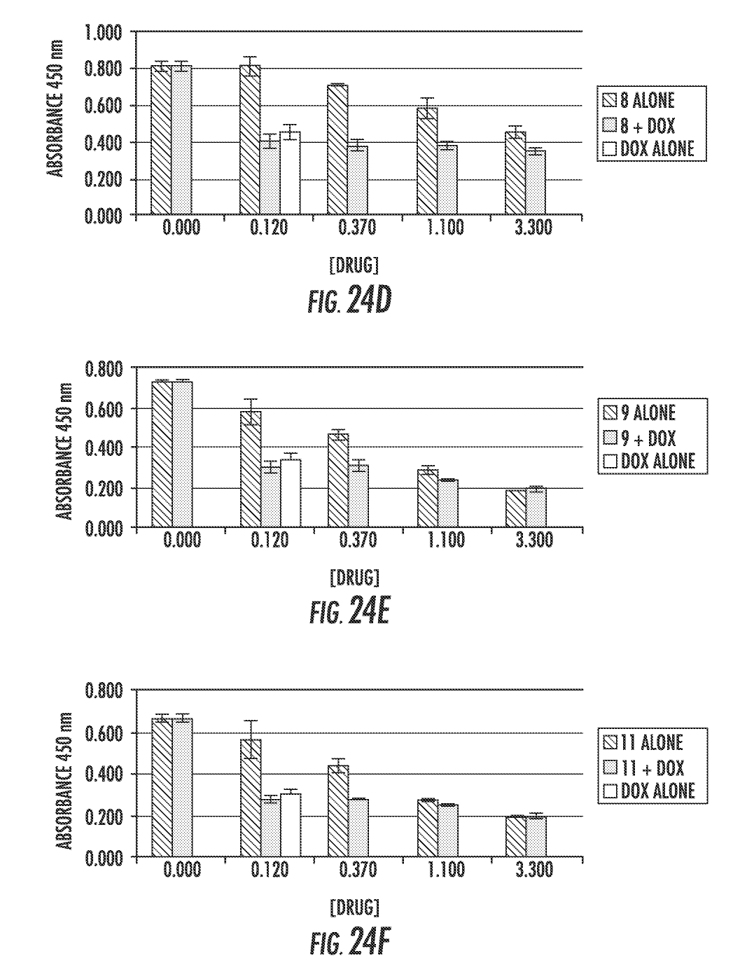

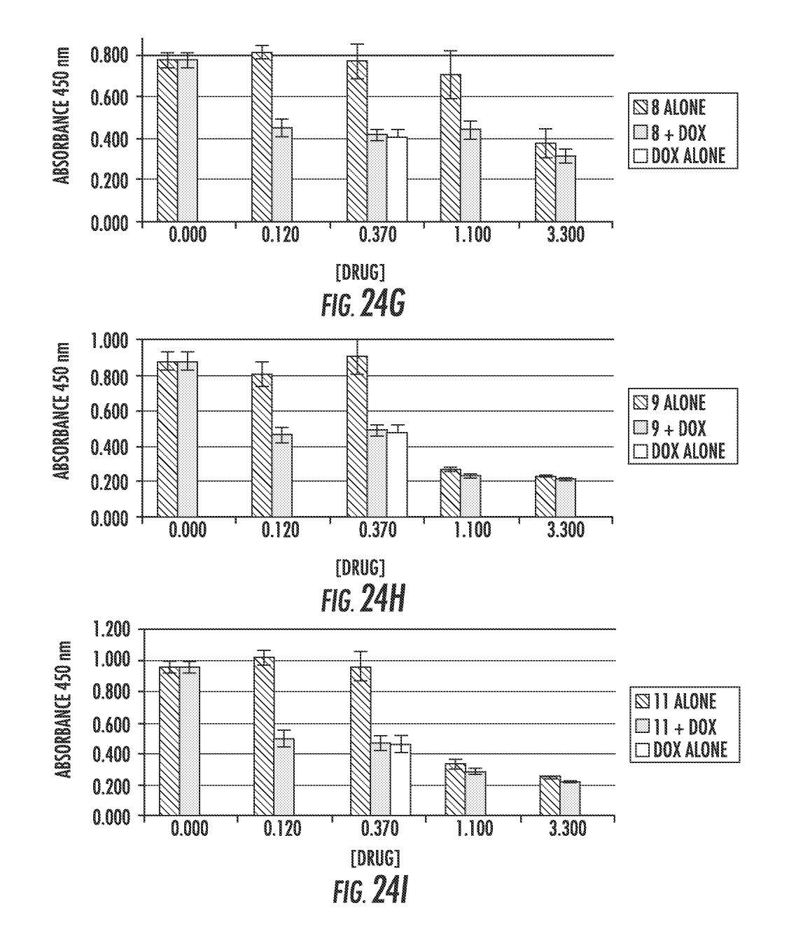

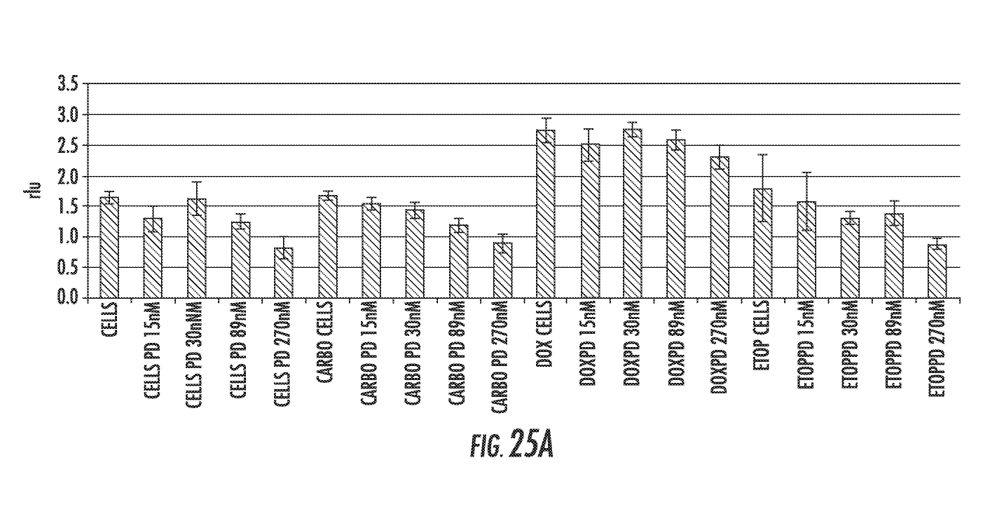

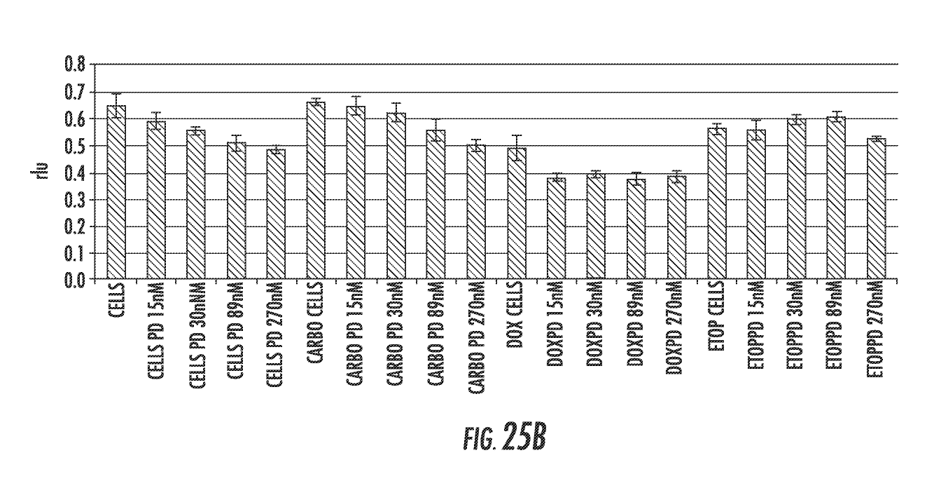

Cdk4/6-dependent cell lines, including telomerized human diploid fibroblasts (H568) and human melanoma cell line WM2664, demonstrated strong, clean and reversible G1-arrest after exposure to the potent and selective Cdk4/6 inhibitors PD0332991 or 2BrIC. See FIGS. 2A-2E. Less selective CDK inhibitors that additionally target CDK1/2, such as compounds 1-6, flavopiridol (FIG. 20A), compound 7 (i.e., R547; FIG. 21A), Roscovitine (FIG. 22A), Genistein and compounds 8-14 (FIGS. 24A-24C) variably produced a G2/M block, intra-S arrest, or cell death (sub-G0) in these cell types. In contrast, an RB-null melanoma line A2058 was, as expected, insensitive to CDK4/6 inhibition, but similarly displayed a G2/M or intra-S arrest and/or cell death after exposure to the less specific CDK inhibitors. The proliferation of seven RB-deficient human small cell lung cancer lines was also resistant to CDK4/6 inhibitors. Thus, the data indicates that structurally distinct, potent and selective Cdk4/6 inhibitors effect a substantially pure (i.e., “clean”) G1-arrest in susceptible cell lines (CDK4/6-dependent cell lines), whereas the cell cycle effects of more global and nonspecific CDK inhibitors are less predictable and associated with cytotoxicity.

### Example 3

### Protection from DNA Damage in Cells Treated with Chemotherapeutic Agents

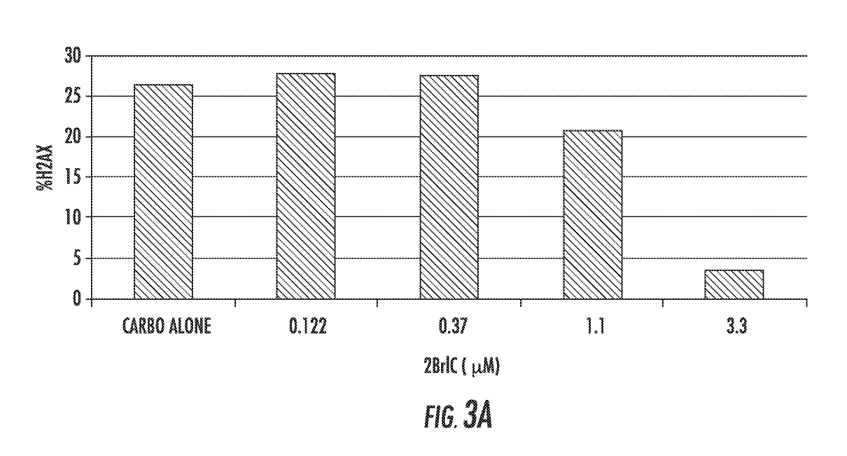

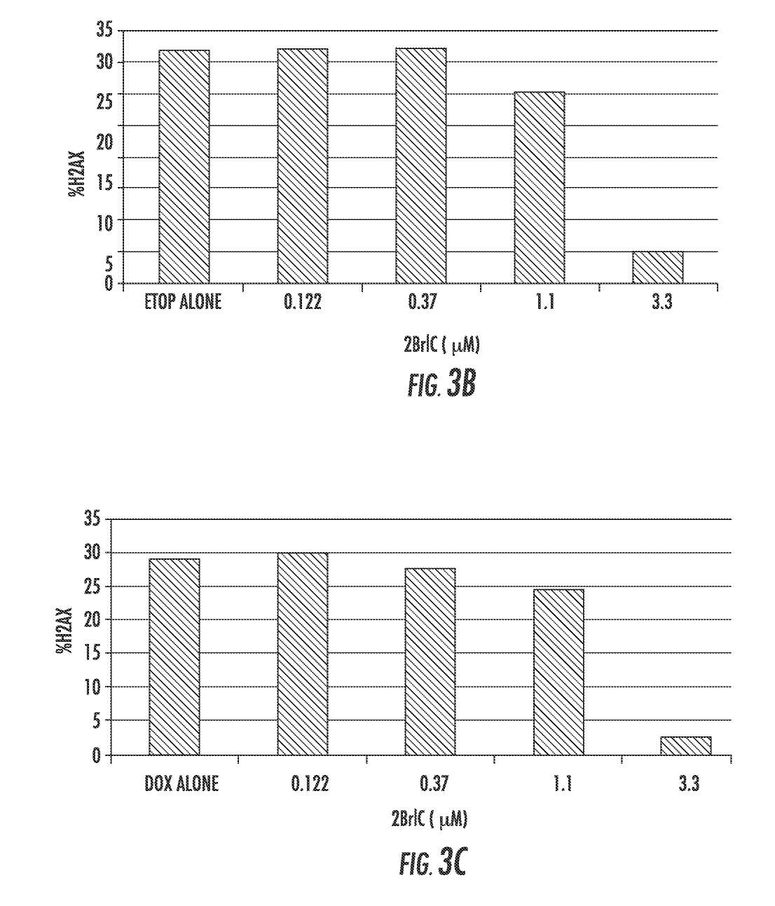

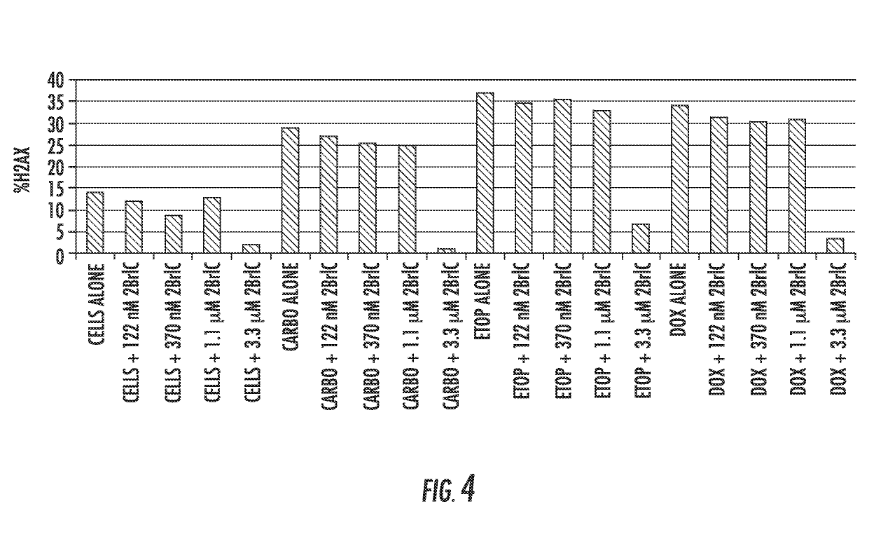

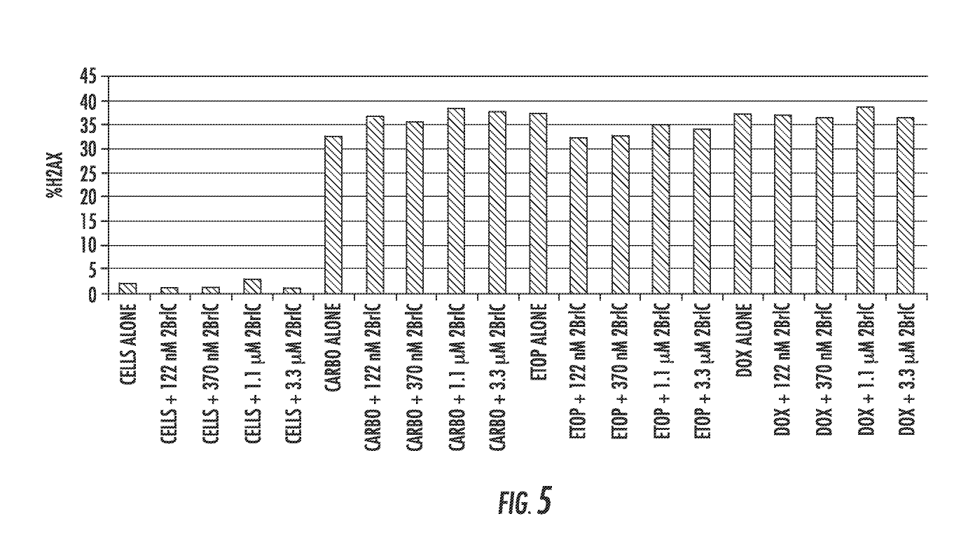

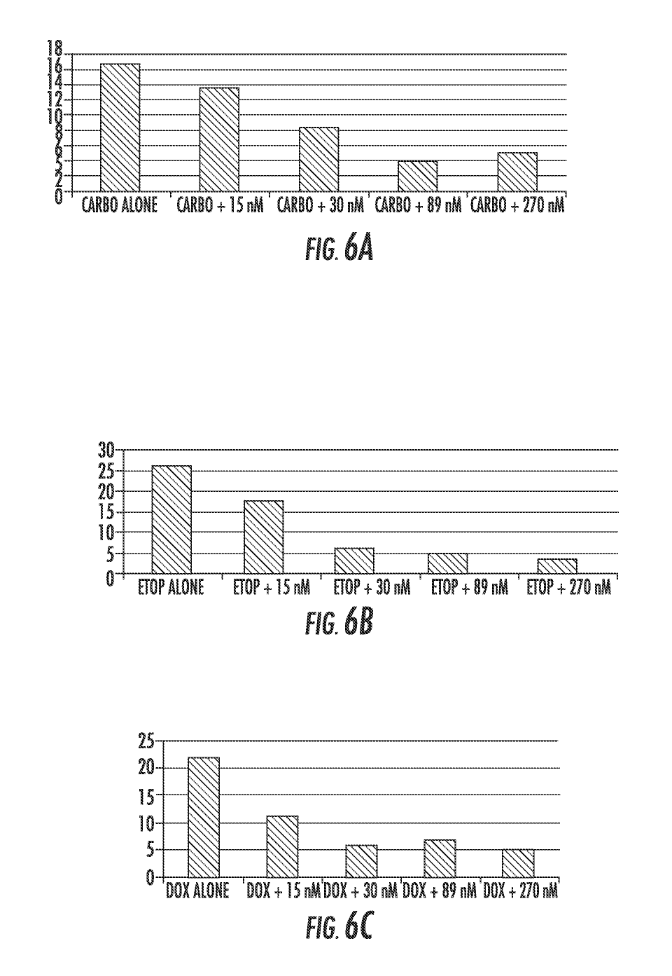

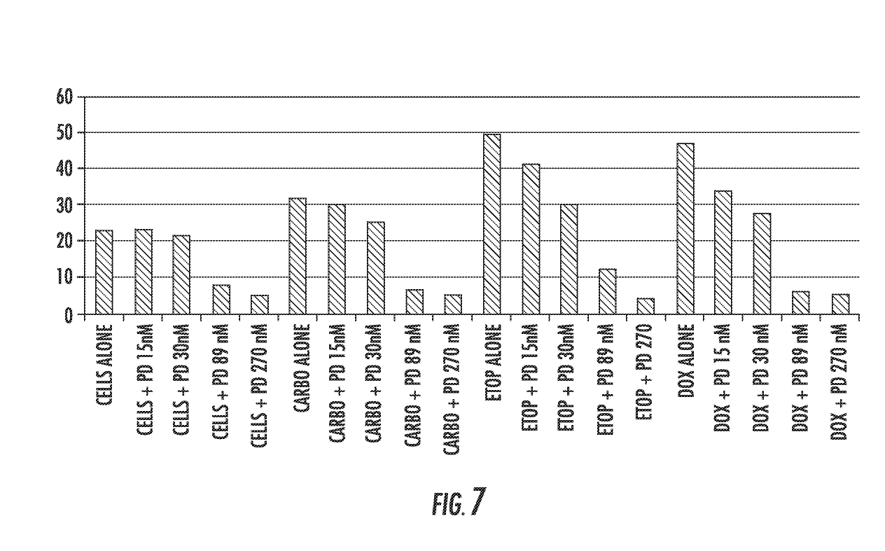

The ability of selective CDK4/6 inhibitors to reduce DNA damage in cells exposed to DNA damaging compounds, such carboplatin, etoposide and doxorubicin, was assayed in a cell based assays as described hereinabove in the Methods section. Carboplatin, etoposide, and doxorubicin caused extensive DNA damage, as measured by γH2AX foci formation in both CDK4/6 dependent and independent cell lines. See FIGS. 3A-3C, 4, and 5. Treatment with PD0332991 (FIGS. 6A-6C, 7, and 8) or 2BrIC (FIGS. 3A-3C, 4, and 5) prior to treatment with carboplatin, etoposide or doxorubicin attenuated γH2AX staining suggesting the G1 arrest induced by PD0332991 and 2BrIC protected the cells from chemotherapy-induced DNA damage.

### Example 4

### Protection from Cytotoxicity in Cells Treated with Chemotherapeutic Agents

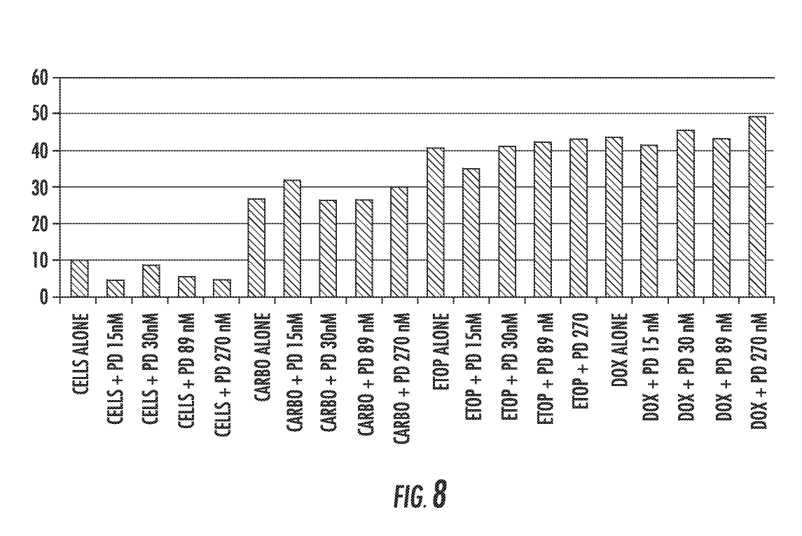

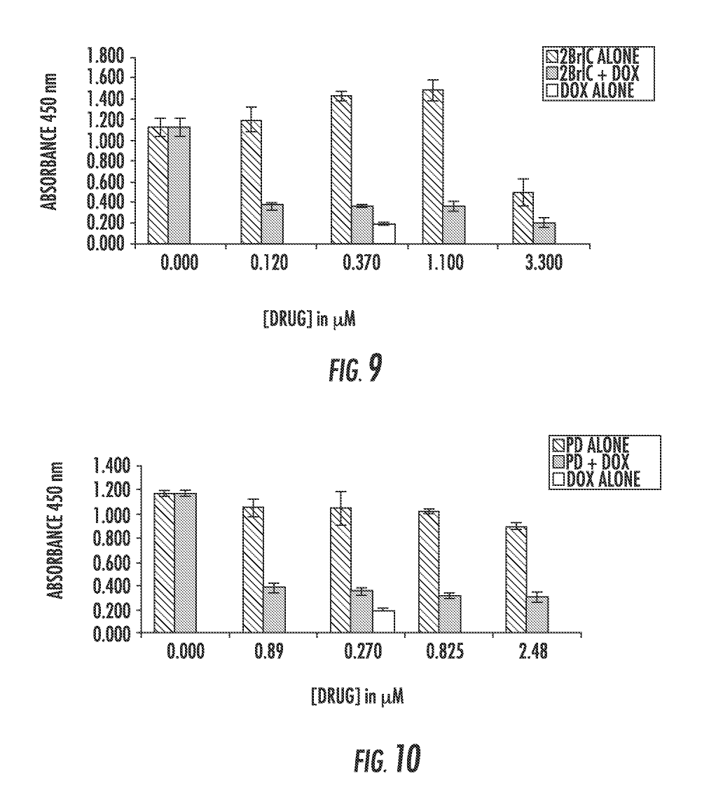

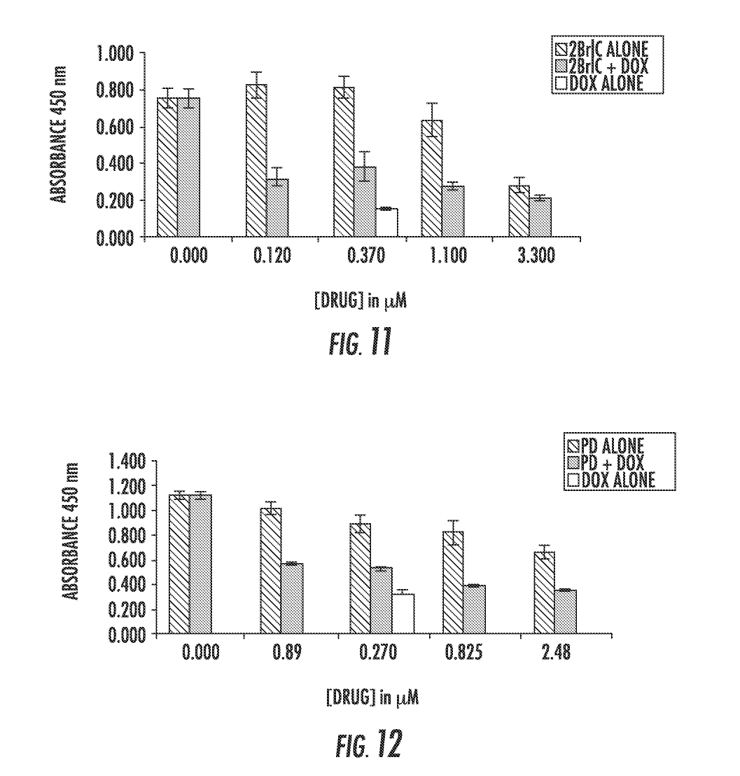

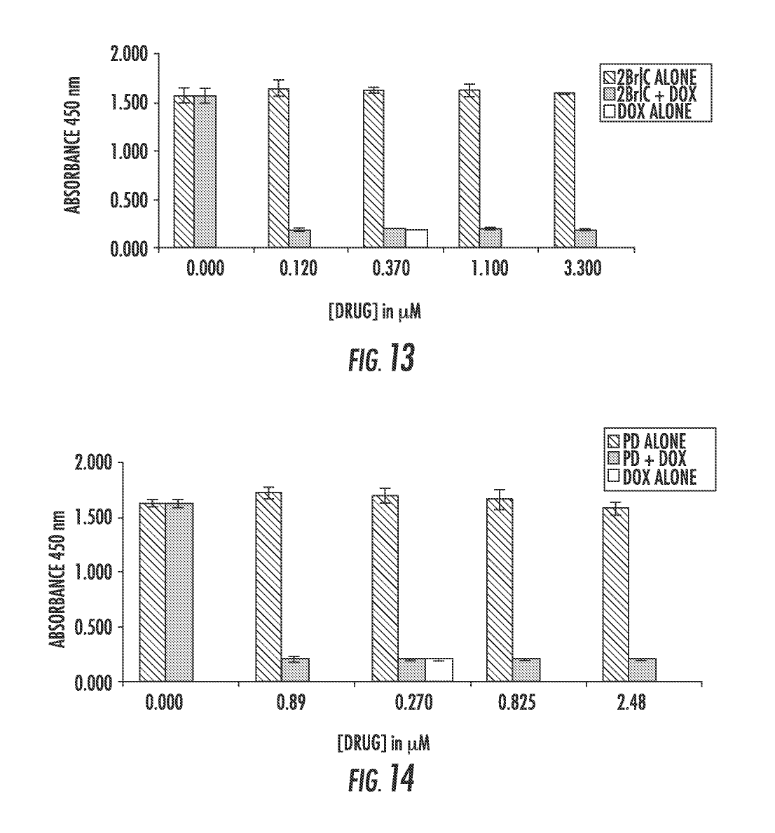

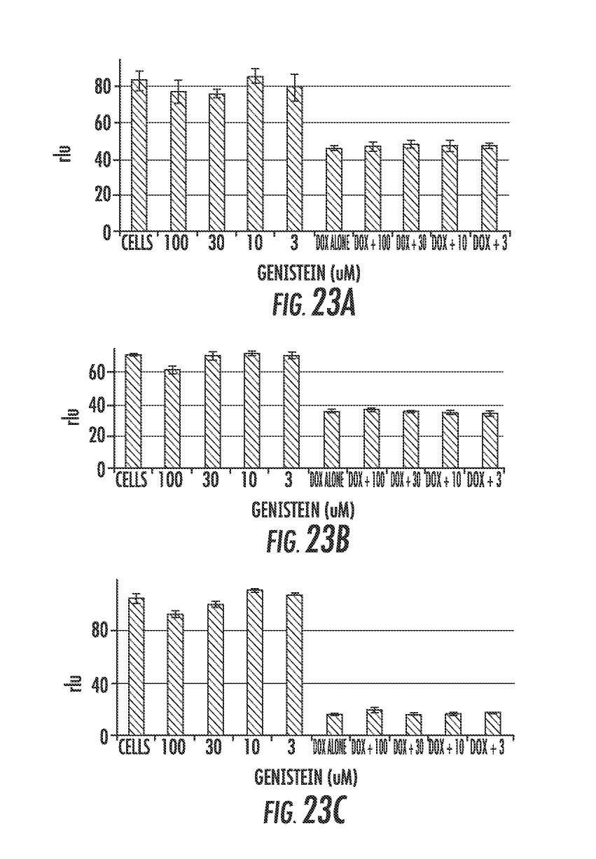

The ability of selective CDK4/6 inhibitors to protect cells from chemotherapy-induced cytotoxicity was assessed in cell based cell proliferation assays as described hereinabove in the Methods section. CDK4/6-dependent and independent cell lines were pretreated with PD332991 and 2BrIC prior to addition of carboplatin, etoposide or doxorubicin. Both PD332991 and 2BrIC provided significant protection of CDK4/6-dependent cells but not the CDK4/6-independent cells. See FIGS. 9-14 and 25A-25C. In contrast, the less selective CDK inhibitors that additionally target CDK1/2, such as flavopiridol (FIGS. 20B-20D), compound 7 (i.e., R547; FIGS. 21B-21D), roscovitine (FIGS. 22B-22D), genistein (FIGS. 23A-23C) and compounds 8, 9, and 11 (FIGS. 24D-24I), and which did not induce a clean G1 arrest in CDK4/6-dependent or independent cells, failed to protect cells from chemotherapy-induced cytotoxicity. The failure of the less selective inhibitors to afford protection suggests that arrest in a phase of the cell cycle other than G1 (e.g. G2/M) does not protect from genotoxic exposure.

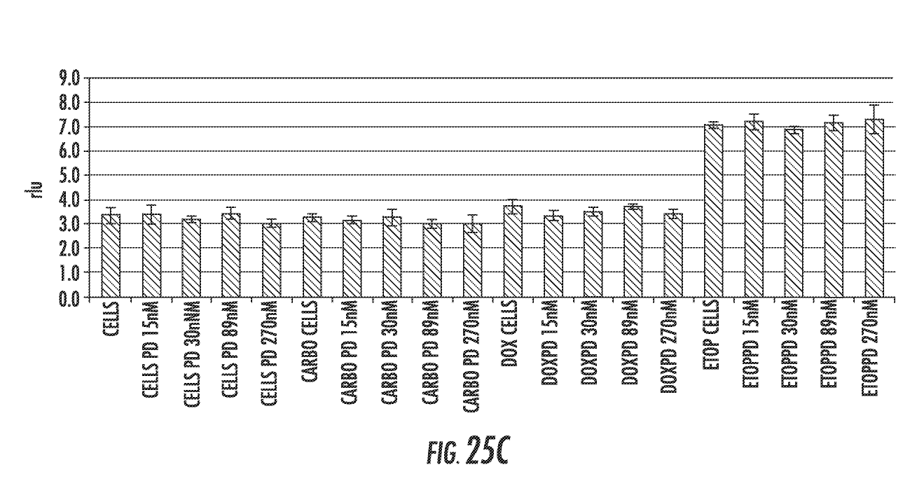

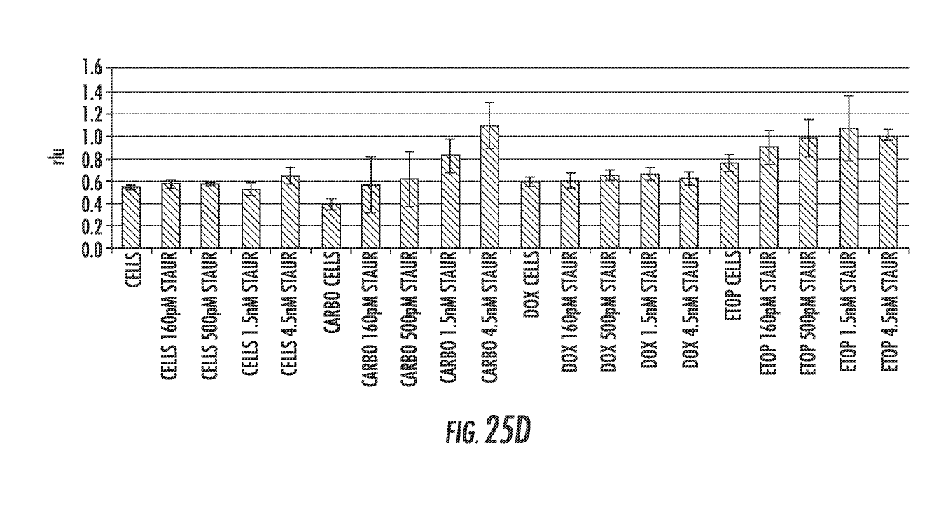

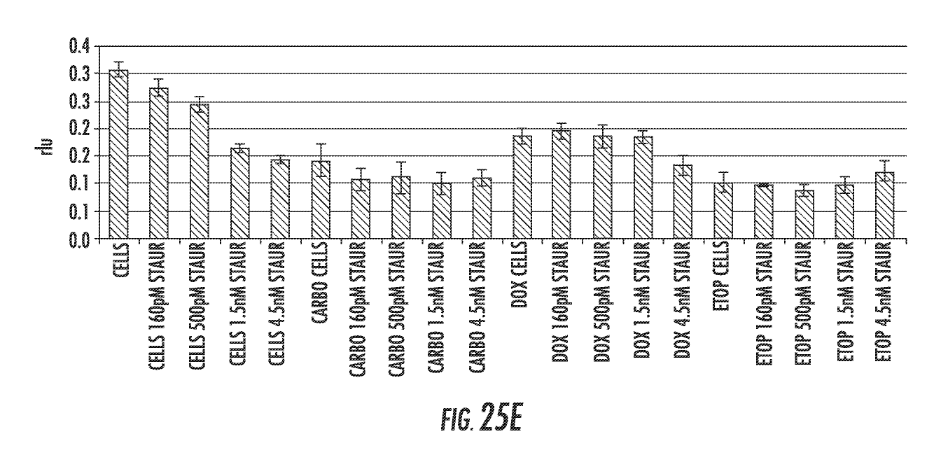

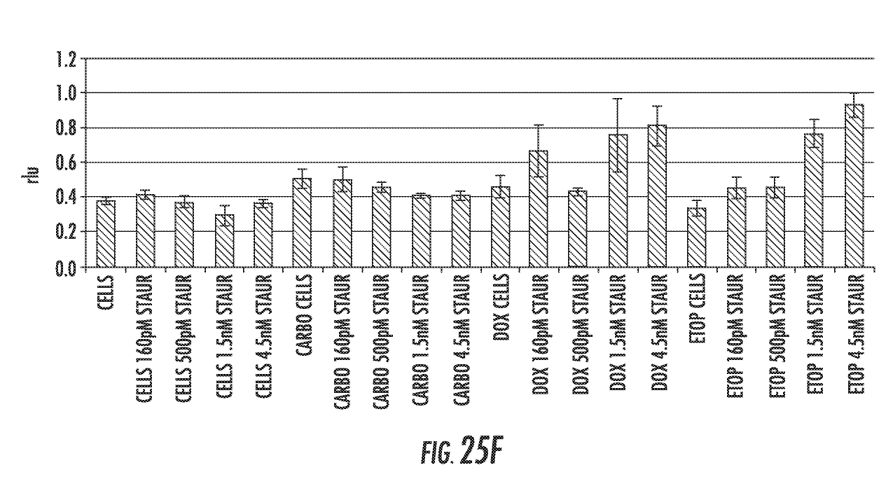

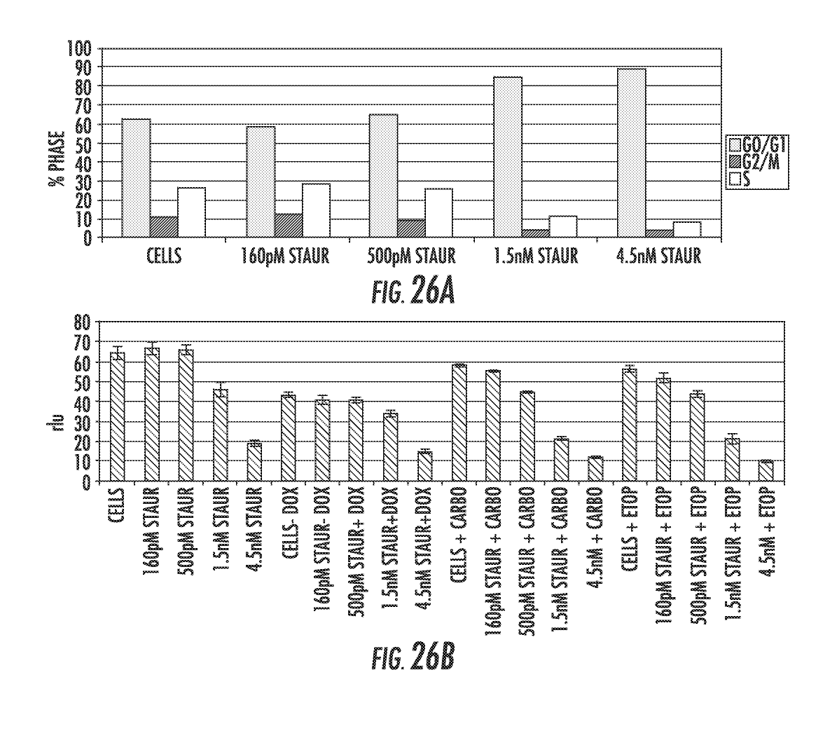

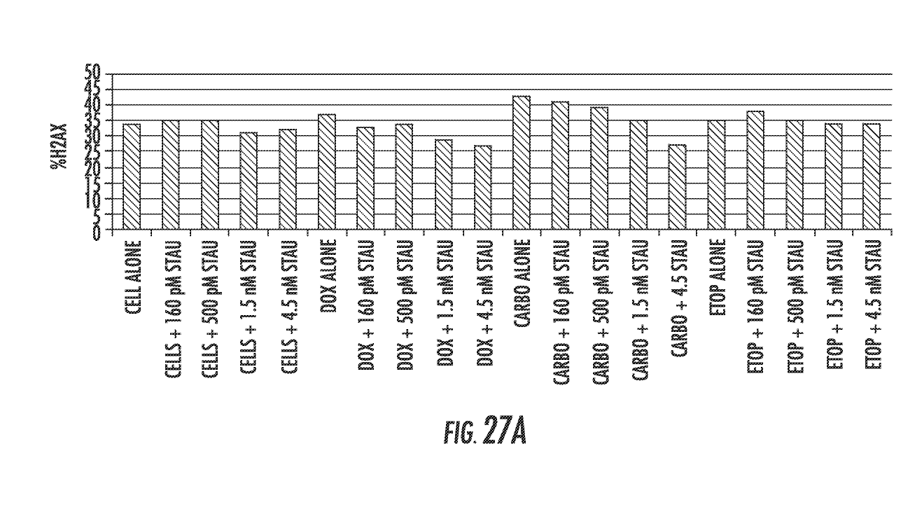

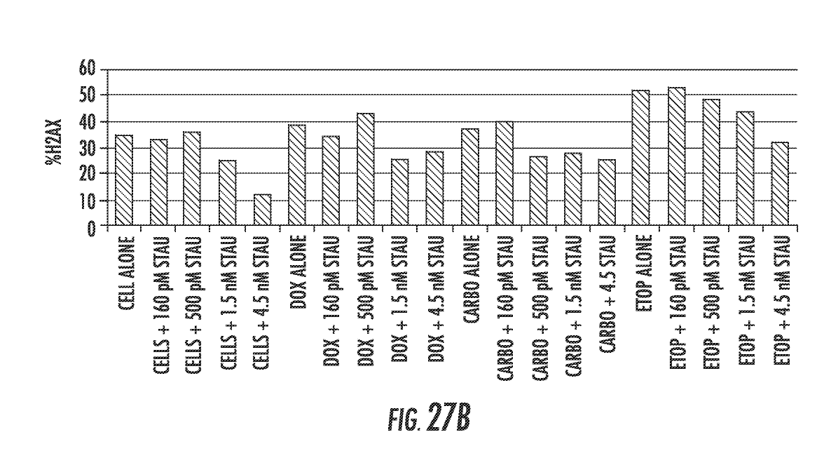

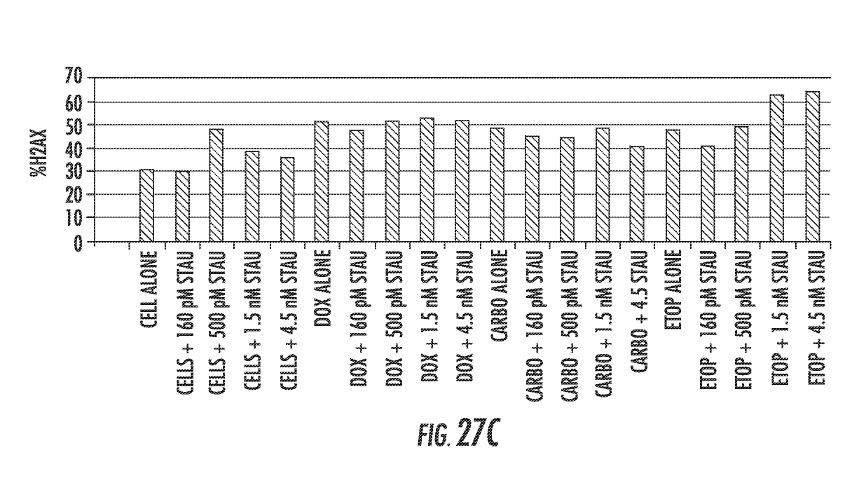

It is important to note that merely being a potent inhibitor of CDK4/6 and providing G1 arrest in some CDK4/6 sensitive cell lines is not sufficient for optimal protection from cytotoxic compounds. In some embodiments, disclosed herein is the use of CDK4/6 inhibitors that are not only potent but highly selective for these kinases, and not other CDK's or other non-CDK kinases. For example, in FIG. 26A, staurosporine, a potent but non-selective CDK4/6 inhibitor, induces a substantially pure G1 arrest in one CDK4/6-dependent cell type, HS68; but this arrest does not provide protection from chemotherapy toxicity. See FIG. 26B. In FIGS. 25D-25F, it is shown that staurosporine treatment enhances cytotoxicity in both WM2664 (CDK4/6-dependent) and A2058 (CDK4/6-independent) cell lines. Likewise, staurosporine does not protect either cdk4/6 dependent or cdk4/6 independent cells from DNA damage as measured by H2AX foci. See FIGS. 27A-27C. In aggregate, these results, therefore, suggest this compound's off-target, CDK4/6-independent effects are inducing cell death in some situations, suggesting that the effects of multi-potent kinase inhibitors such as staurosporine with regard to chemoprotection will be variable and cell-type dependent: affording protection in vitro in some cell types where the major effect of these drugs is to directly or indirectly induce G1 arrest (see Chen et al., J. Natl. Cancer Inst., 92, 1999-2008 (2000) and cell death or other undesirable outcomes in cell types where the off-target effects are detrimental to cell survival. In some embodiments of the presently disclosed subject matter, a multi-assay screen (e.g., cell cycle arrest and H2AX protection and/or cellular growth at 7 days) can be performed to determine effective in vivo chemoprotectants. In such a screen, staurosporine fails because of these off-target and inconsistent effects.

Further, these off-target effects produce toxicity and enhanced chemotherapy sensitivity when used in vivo, and these toxicities can preclude the use of multi-potent kinase inhibitors for clinical chemoprotection. Disclosed herein in accordance with some embodiments of the presently disclosed subject matter, the unexpected finding that the exquisite specificity of selective CDK4/6 inhibitors for a fraction of proliferating cells (i.e., early HSPC), is such that such compounds can be employed for clinical chemotherapy protection in vivo without causing dose-limiting toxicities.

### Example 5

### In Vivo Chemoprotection

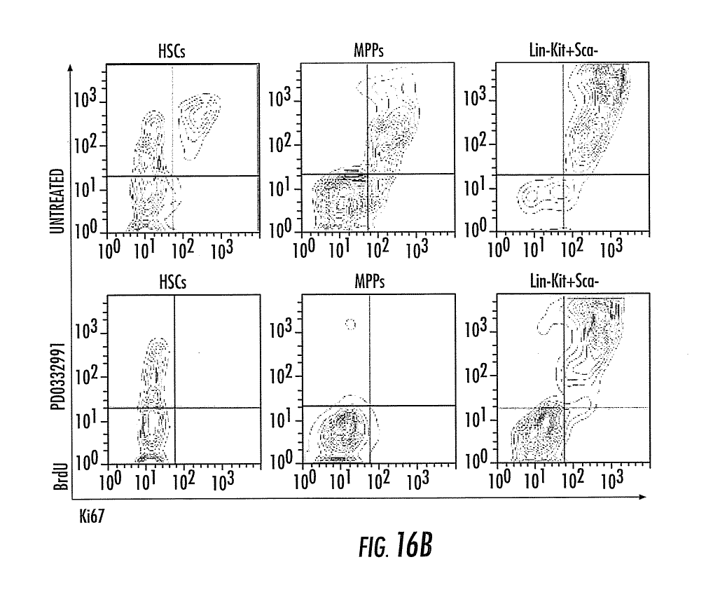

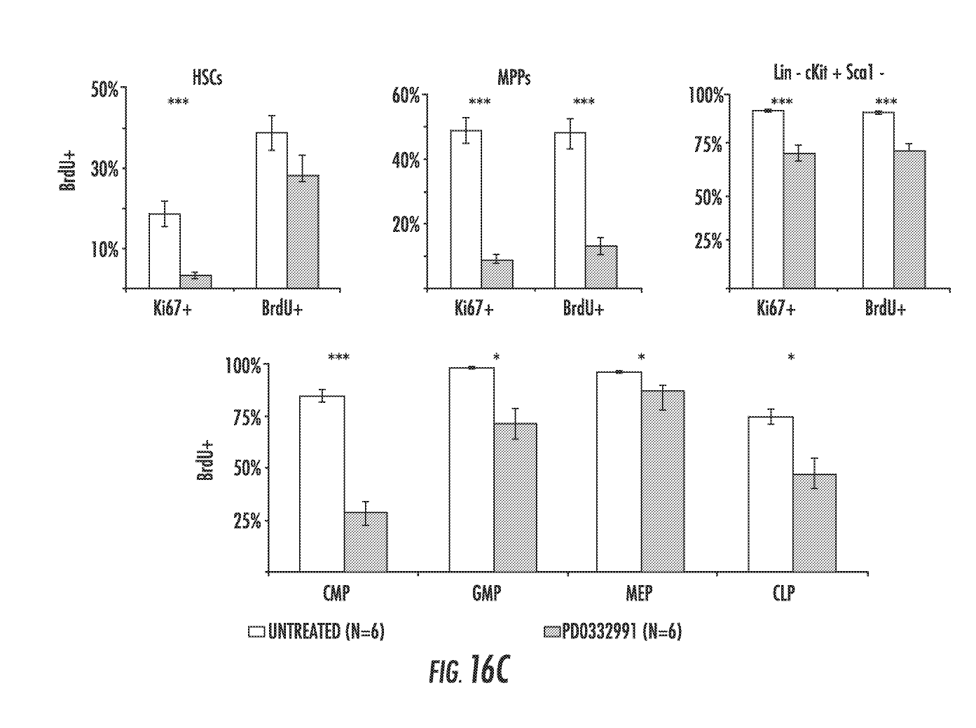

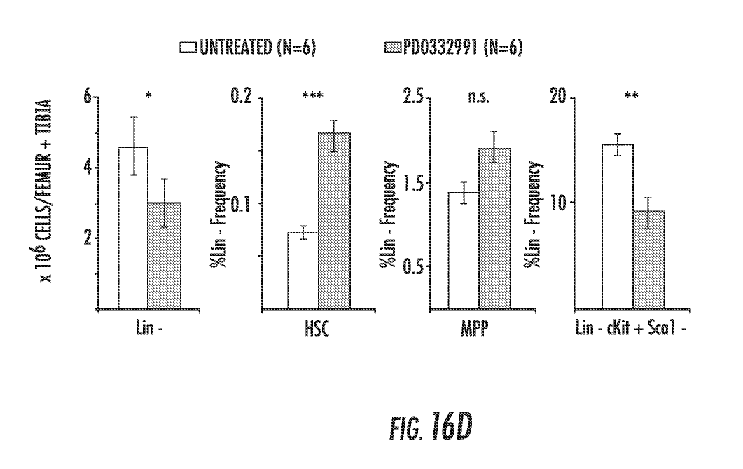

The ability to provide PQ in vivo using selective CDK4/6 inhibitors was assessed. PD0332991, which is orally bioavailable, was administered to adult wild-type C57BI/6 mice by oral gavage. Proliferation of hematopoietic stem cells (HSC; Lin-Kit+Sca1+CD48-CD150+) measured by Ki67 expression and incorporation of BrdU over 24 hours was slow (see FIGS. 16A-16D), comparable to prior estimates. See Passeque et al., 2005; Wilson et al., 2008; and Kiel et al., 2007. PD0332991 treatment for 48 hours significantly decreased the frequency of HSC doubly positive for expression of Ki67 and BrdU (FIG. 16B), with more pronounced effects on Ki67 expression. A more pronounced inhibition of proliferation was noted in the more rapidly proliferating multipotent progenitor cell compartment (MPP; Lin-Kit+Sca1+CD48-CD150−) (FIG. 16B-16C). Oligopotent progenitors (Lin-Kit+Sca1−) demonstrated modest inhibition of proliferation (FIG. 16C), with the strongest effects seen in common myeloid progenitors (CMP) and common lymphocyte progenitors (CLP) compared to weaker effects in the more differentiated granulocyte-monocyte progenitors (GMP) and megakaryocyte-erythroid progenitors (MEP; FIGS. 16B-16C). In contrast to these effects on early HSPC, no change in proliferation was noted in the more fully differentiated Lin-Kit−Sca1− and Lin+ cells, though these fractions are heterogeneous and effects on subpopulations may be obscured.

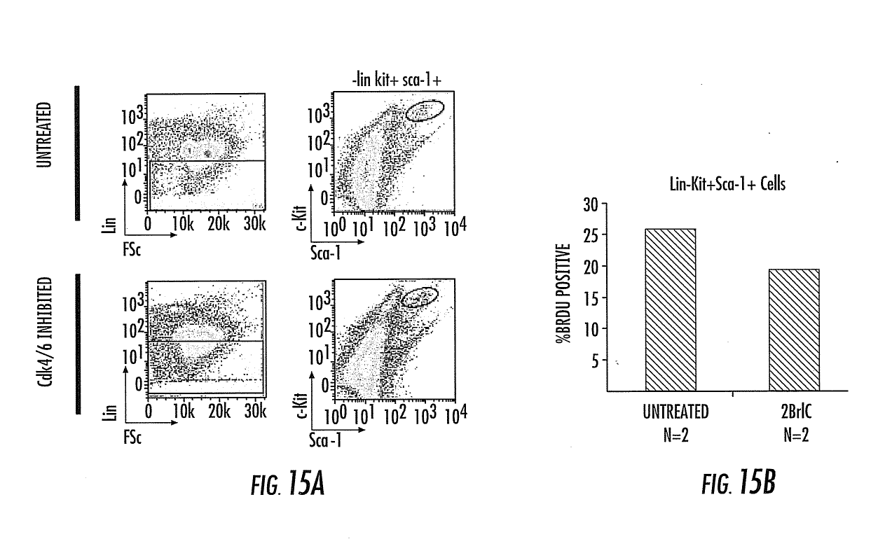

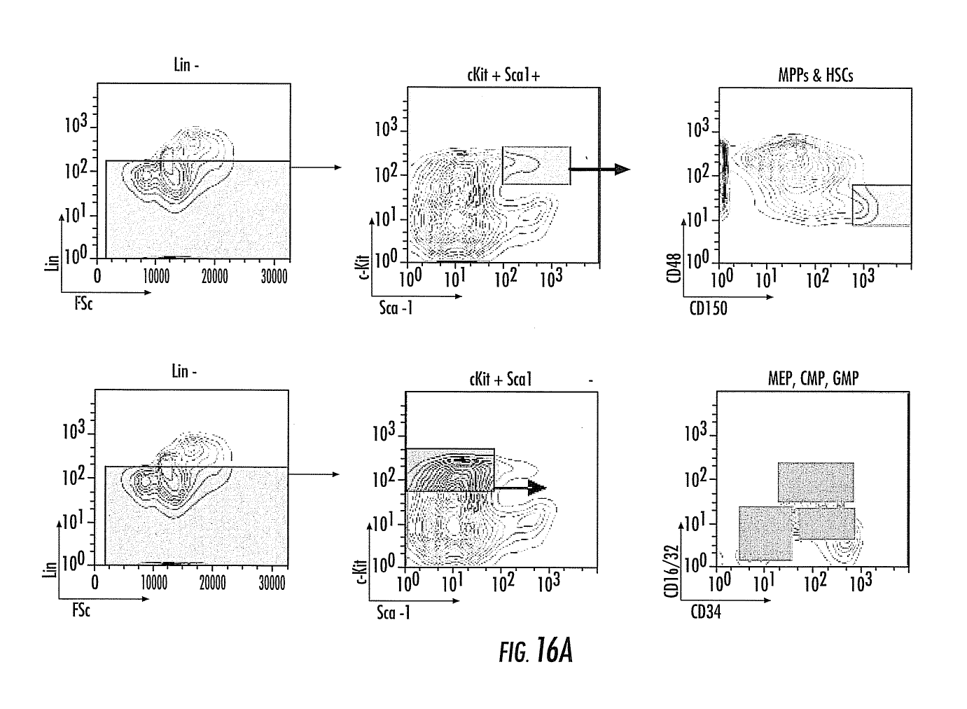

2BrIC was solubilized and given by oral gavage 2 hours prior to BrdU injection with an additional dose given at the time of BrdU injection. 2BrIC inhibited the incorporation of BrdU into Lin-Kit+Sca1+ cells relative to mice treated with formulation alone. See FIGS. 15A-15B.

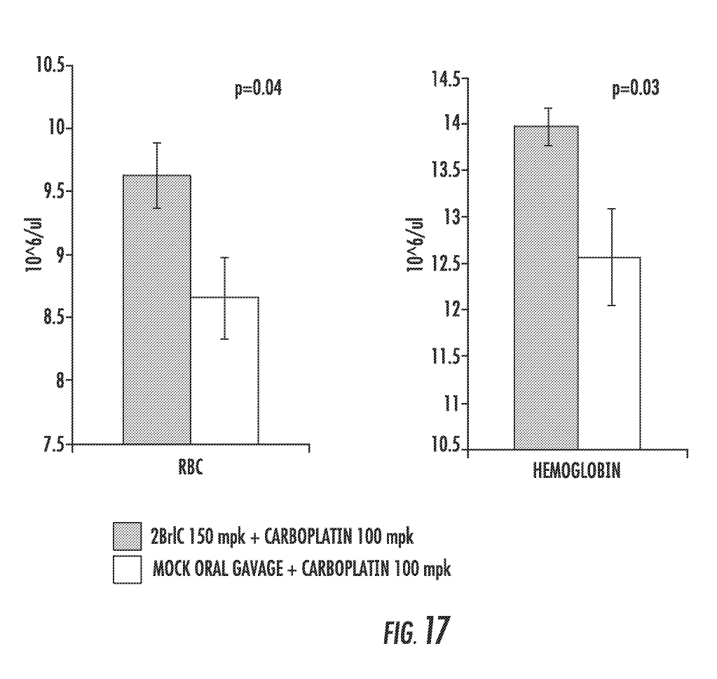

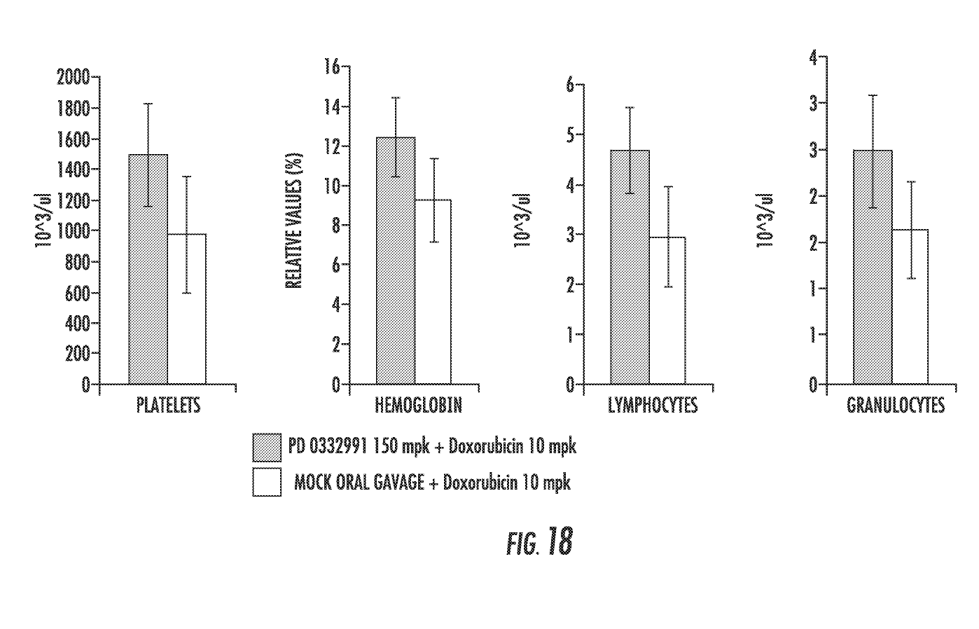

To determine whether selective CDK4/6 inhibitors could protect blood counts in mice exposed to chemotherapeutic agents, blood cell counts were studied in mice treated with PD332991. All four lineages (platelets, hemoglobin, lymphocytes, and granulocytes) were protected in mice pretreated with PD332991 followed by doxorubicin (see FIG. 18) or carboplatin (see FIG. 19).

A decrease in the erythroid, platelet and myeloid (monocyte+granulocyte) lineages has been observed upon admistration of PD0332991 along with an increase of such lineages upon cessation of PD0332991 in tumor bearing mice (see Ramsey et al., Cancer Res., 67, 4732-4741 (2007); and Fry et al., Mol. Cancer Ther., 3, 1427-1438 (2004)) and in human patients with malignancies (see O'Dwyer et al., “A Phase I does escalation trial of a daily oral CDK4/6 Inhibitor PD 0332991” in American Society of Clinical Oncology (ASCO, Chicago, Ill., 2007)) that were serially treated with PD0332991. The noted decreases might be expected to enhance the adverse effects of a cytotoxic compound administered in chemotherapy. However, unexpectedly as shown herein, hematopoietic cells are protected from adverse effects.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method of reducing or preventing the effects of a cytotoxic compound on healthy cells in a subject who has been exposed to, shall be exposed to, or is at risk of incurring exposure to a cytotoxic compound, wherein said healthy cells are hematopoietic stem cells or hematopoietic progenitor cells, the method comprising administering to the subject an effective amount of an inhibitor compound, or a pharmaceutically acceptable form thereof, wherein the inhibitor compound selectively inhibits cyclin-dependent kinase 4 (CDK4) and/or cyclin-dependent kinase 6 (CDK6).

2. The method of claim 1, wherein the inhibitor compound selectively inhibits both cyclin-dependent kinase 4 (CDK4) and cyclin-dependent kinase 6 (CDK6).

3. The method of claim 1, wherein the inhibitor compound is a non-naturally occurring compound.

4. The method of claim 1, wherein the inhibitor compound is substantially free of off-target effects.

5. The method of claim 4, wherein the off-target effects are one or more of the group consisting of long term toxicity, anti-oxidant effects, estrogenic effects, tyrosine kinase inhibition, inhibition of cyclin-dependent kinases (CDKs) other than cyclin-dependent kinase 4/6 (CDK4/6), and cell cycle arrest in CDK4/6-independent cells.

6. The method of claim 1, wherein the inhibitor compound selectively induces G1 arrest in cyclin-dependent kinase 4 (CDK4)- and/or cyclin-dependent kinase 6 (CDK6)-dependent cells.

7. The method of claim 6, wherein the inhibitor compound induces substantially pure G1 arrest in cyclin-dependent kinase 4 (CDK4)- and/or cyclin-dependent kinase 6 (CDK6)-dependent cells.

8. The method of claim 1, wherein the inhibitor compound is selected from the group consisting of a pyrido[2,3-d]pyrimidine, a triaminopyrimidine, an aryl[a]pyrrolo[3,4-c]carbazole, a nitrogen-containing heteroaryl-substituted urea, a 5-pyrimidinyl-2-aminothiazole, a benzothiadiazine, and an acridinethione.

9. The method of claim 8, wherein the pyrido[2,3-d]pyrimidine is a pyrido[2,3-d]pyrimidin-7-one or a 2-amino-6-cyano-pyrido[2,3-d]pyrimidin-4-one.

10. The method of claim 9, wherein the pyrido[2,3-d]pyrimidin-7-one is a 2-(2′-pyridyl)amino pyrido[2,3-d]pyrimidin-7-one.

11. The method of claim 10, wherein the pyrido[2,3-d]pyrimidin-7-one is 6-acetyl-8-cyclopentyl-5-methyl-2-(5-piperazin-1-yl-pyridin-2-ylamino)-8H-pyrido-[2,3-d]pyrimidin-7-one.

12. The method of claim 8, wherein the aryl[a]pyrrolo[3,4-c]carbazole is selected from the group consisting of a napthyl[a]pyrrolo[3,4-c]carbazole, an indolo[a]pyrrolo[3,4-c]carbazole, a quinolinyl[a]pyrrolo[3,4-c]carbazole, and an isoquinolinyl[a]pyrrolo[3,4-c]carbazole.

13. The method of claim 12, wherein the aryl[a]pyrrolo[3,4-c]carbazole is 2-bromo-12,13-dihydro-5H-indolo[2,3-a]pyrrolo[3,4]-carbazole-5,6-dione.

14. The method of claim 1, wherein the subject is a mammal.

15. The method of claim 1, wherein the inhibitor compound is administered to the subject by one of the group consisting of oral administration, topical administration, intranasal administration, inhalation, and intravenous administration.

16. The method of claim 1, wherein the inhibitor compound is administered to the subject prior to exposure to the cytotoxic compound, during exposure to the cytotoxic compound, after exposure to the cytotoxic compound or any combination thereof.

17. The method of claim 16, wherein the inhibitor compound is administered to the subject 24 hours or less prior to exposure to the cytotoxic compound.

18. The method of claim 16, wherein the inhibitor compound is administered to the subject 24 hours or more following exposure to the cytotoxic compound.

19. The method of claim 1, wherein the cytotoxic compound is a DNA damaging compound.

20. The method of claim 1, wherein the healthy cells are selected from the group consisting of long term hematopoietic stem cells (LT-HSCs), short term hematopoietic stem cells (ST-HSCs), multipotent progenitors (MPPs), common myeloid progenitors (CMPs), common lymphoid progenitors (CLPs), granulocyte-monocyte progenitors (GMPs), and megakaryocyte-erythroid progenitors (MEPs).

21. The method of claim 1, wherein administration of the inhibitor compound provides temporary pharmacologic quiescence in hematopoietic stem and progenitor cells.

22. The method of claim 1, wherein the subject has undergone, is undergoing, or is expected to undergo medical treatment with a cytotoxic compound to treat a disease.

23. The method of claim 22, wherein administration of the inhibitor compound does not affect growth of diseased cells.

24. The method of claim 22, wherein the disease is cancer.

25. The method of claim 24, wherein the cancer is characterized by one or more of the group consisting of increased activity of cyclin-dependent kinase 1 (CDK1), increased activity of cyclin-dependent kinase 2 (CDK2), loss or absence of retinoblastoma tumor suppressor protein (RB), high levels of MYC expression, increased cyclin E and increased cyclin A.

26. The method of claim 22, wherein administration of the inhibitor compound allows for a higher dose of the cytotoxic compound to be used to treat the disease than the dose that would be used in the absence of administration of the inhibitor compound.

27. The method of claim 1, wherein the subject has been accidentally exposed to the cytotoxic compound or to an overdose of the cytotoxic compound.

28. The method of claim 1, wherein the method is free of long-term hematologic toxicity.

29. The method of claim 1, wherein administration of the inhibitor compound results in reduced anemia, reduced lymphopenia, reduced thrombocytopenia, or reduced neutropenia compared to that expected after exposure to the cytotoxic compound in the absence of administration of the inhibitor compound.

30. A method for screening a compound for use in preventing the effects of a cytotoxic agent in a healthy cell, the method comprising:
contacting a cyclin-dependent kinase 4 (CDK4) and/or cyclin-dependent kinase 6 (CDK6)-dependent cell population with a test compound for a period of time;
performing cell cycle analysis of the cell population; and
selecting a test compound that selectively induces G1 arrest in the cell population.

31. The method of claim 30, wherein the cyclin-dependent kinase 4 (CDK4) and/or cyclin-dependent kinase 6 (CDK6)-dependent cell population comprises telomerized human diploid fibroblast cells or melanoma cells lacking INK4a/ARF.

32. The method of claim 30, wherein the cell cycle analysis is performed using one or more of the techniques selected from flow cytometry, fluorimetry, cell imaging, and fluorescence spectroscopy.

33. The method of claim 30, wherein the cell cycle analysis comprises labelling the cell population with one or more labeling agents selected from the group consisting of 5-bromo-2-deoxyuridine (BrdU) and propidium iodide.

34. The method of claim 30, wherein the method further comprises:
contacting a second cell population with the test compound that selectively induces G1 arrest in cyclin-dependent kinase 4 (CDK4) and/or cyclin-dependent kinase 6 (CDK6)-dependent cells for a period of time, wherein the second cell population comprises CDK4- and/or CDK6-independent cells;
performing cell cycle analysis in the second cell population; and
selecting a test compound that is free of selective induction of G1 arrest in the second cell population.

35. The method of claim 34, wherein the second cell population is a cancer cell line.

36. The method of claim 34, wherein the second cell population is retinoblastoma tumor suppressor protein (RB)-null.

37. The method of claim 30, wherein the method further comprises confirming the preventative ability of the test compound by assessing the ability of the compound to reduce DNA damage, to maintain cell viability, or both in an ex vivo cell population contacted with a cytotoxic agent.

38. The method of claim 37, wherein DNA damage in the cell population is assessed by performing a gamma-H2AX assay.

39. The method of claim 37, wherein cell viability is assessed by performing a cell proliferation assay.

40. The method of claim 37, wherein the cytotoxic agent is a DNA damaging compound.

41. The method of claim 40, wherein the DNA damaging compound is selected from the group consisting of doxorubicin, etoposide and carboplatin.
